(19) United States
(12) Patent Application Publication
(10) Pub. No.: US 2019/0185507 A1
(43) Pub. Date: Jun. 20, 2019

(54) Sofosbuvir Derivatives for the Treatment of Hepatitis C

(71) Applicant: Sandoz AG, Basel (CH)

(72) Inventors: George Moore, Holzkirchen (DE); Desiree Strych, Kundl (AT); Michael Papp, Kundl (AT); Olga Schoene, Kundl (AT); Thorsten Wilhelm, Kundl (AT); Hannes Lengauer, Kundl (AT)

(73) Assignee: Sandoz AG, Basel (CH)

(21) Appl. No.: 16/325,834

(22) PCT Filed: Aug. 17, 2017
(86) PCT No.: PCT/EP2017/070832

§ 371 Date: Feb. 15, 2019

(30) Foreign Application Priority Data
Aug. 19, 2016 (EP) 16184953.4

(51) Int. Cl. C07H 19/10; C07H 1/02; C07F 9/572

(52) U.S. Cl. CPC C07H 19/10; C07B 2200/13; C07F 9/572; C07H 1/02

(57) ABSTRACT

The present invention relates to novel compounds for the treatment of Hepatitis C.

### FIELD OF THE INVENTION

The present invention relates to new compounds for the treatment of Hepatitis C.

### BACKGROUND

Sofosbuvir according to formula (A)

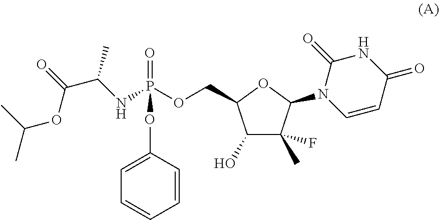 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

with IUPAC name (S)-isopropyl 2-(((S)-(((2R,3R,4R,5R)-5-(2,4-dioxo-3,4-dihydropyrimidin-1(2H)-yl)-4-fluoro-3-hydroxy-4-methyltetrahydrofuran-2-yl)methoxy)(phenoxy)phosphoryl)-amino)propanoate is a drug inhibiting the RNA polymerase used by the Hepatitis C virus to replicate its RNA. WO2008/121634 describes, among a myriad of other compounds, Sofosbuvir, and its crystalline forms, preparation and pharmaceutical compositions comprising the same are described in, among others, WO2010/135569, WO2011/123645, WO2013/082003 and WO2015/099989.

Interestingly, none of the above-referenced documents clearly and unambiguously disclose the compounds of the present invention. For example, WO2008/121634, which includes over 500 pages full of tables disclosing an enormous amount of compounds, does not disclose the possibility of an n-propyl substituent in the amino acid ester moiety of the tabulated compounds. Similarly, Sofia et al. (J. Med. Chem. 2010, 53, 7202), which examined the activities of Sofosbuvir and a number of related compounds, does not disclose an n-propyl substituent in the amino acid ester moiety of the examined compounds, either.

Thus, even though Sofosbuvir has been successful in combatting the Hepatitis C virus and improving the lives of many HCV patients around the world, there is still the need for new compounds capable of fighting the Hepatitis C virus which show high efficacy, are well tolerated by patients, show little or no side effects and can be produced industrially in a cost-competitive and high-yielding manner.

The present invention therefore relates to new compounds which show the above-mentioned characteristics, as well as suitable processes for their preparation, compositions comprising said compounds as well as their use.

### BRIEF DESCRIPTION OF THE FIGURES

FIG. 1: shows the efficacy of the AADs of Sofosbuvir and n-Propyl-Sofosbuvir (compound I″a) on HCV production.

FIG. 2: shows the infection scheme to evaluate the efficacy of Sofosbuvir and n-Propyl-Sofosbuvir (compound I″a) against HCV.

FIG. 3: shows the extension of the concentrations of Sofosbuvir and n-Propyl-Sofosbuvir (compound I″a) to lower doses.

FIG. 4: shows the reduction of the viral titer in the presence of Sofosbuvir and n-Propyl-Sofosbuvir (compound I″a).

FIG. 5: shows the infection scheme to evaluate the efficacy of Sofosbuvir and n-Propyl-Sofosbuvir (compound I″a) against HCV.

FIG. 6: depicts two treatment cycles to test for the efficacy of Sofosbuvir and n-Propyl-Sofosbuvir (compound I″a).

FIG. 7: shows the quantification of the viral load after two treatment cycles with Sofosbuvir and n-Propyl-Sofosbuvir (compound I″a).

FIG. 8: illustrates the efficacy of Sofosbuvir and n-Propyl-Sofosbuvir (compound I″a) in reducing the viral titer after two applications.

FIG. 9: illustrates a representative PXRD of crystalline compound (I″a) (n-Propyl-Sofosbuvir) of the present invention. The x-axis shows the scattering angle in °2-theta, the y-axis shows the intensity of the scattered X-ray beam in counts of detected photons.

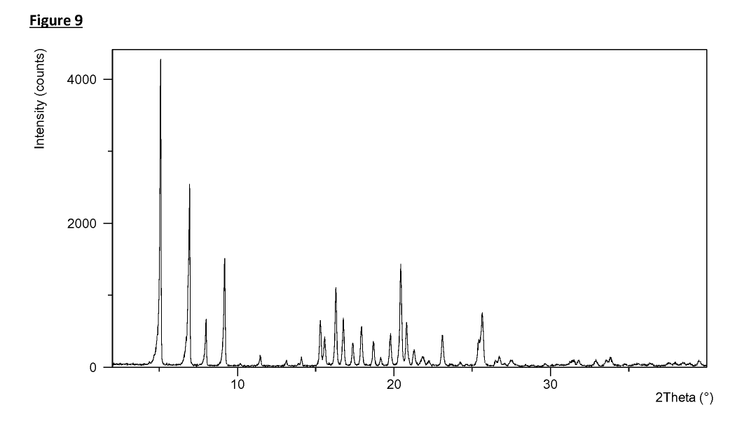

FIG. 10: illustrates a representative DSC curve of crystalline compound (I″a) (n-Propyl-Sofosbuvir) of the present invention. The x-axis shows the temperature in degree Celsius (° C.), the y-axis shows the heat flow rate in Watt per gram (W/g) with endothermic peaks going up.

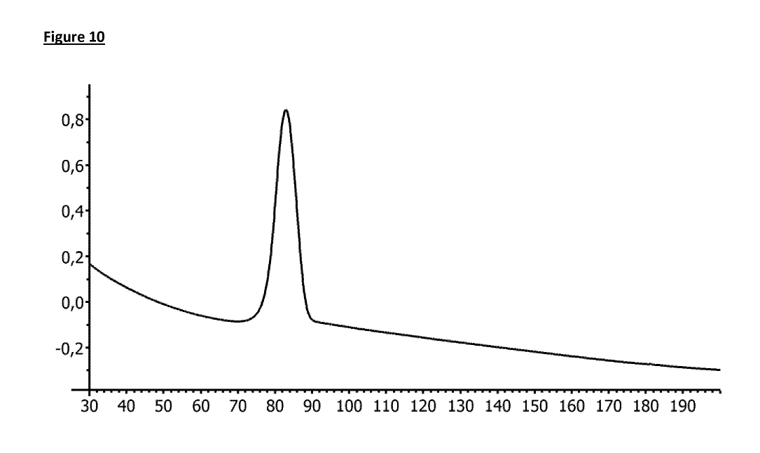

FIG. 11: illustrates a representative TGA curve of crystalline compound (I″a) (n-Propyl-Sofosbuvir) of the present invention. The x-axis shows the temperature in degree Celsius (° C.), the y-axis shows the mass (loss) of the sample in weight percent (w-%).

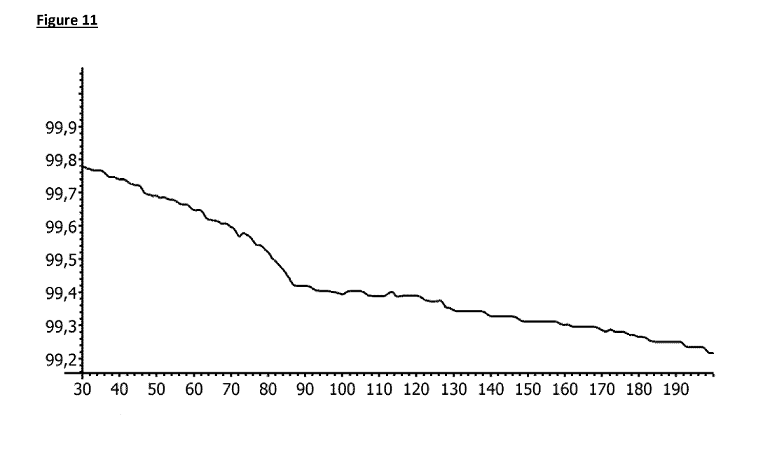

FIG. 12: illustrates representative GMS isotherms of crystalline compound (I″a) (n-Propyl-Sofosbuvir) of the present invention in the range of from 0 to 95% relative humidity. The x-axis displays the relative humidity in percent (%) measured at a temperature of (25.0±0.1)° C., the y-axis displays the equilibrium mass change in weight percent (w-%).

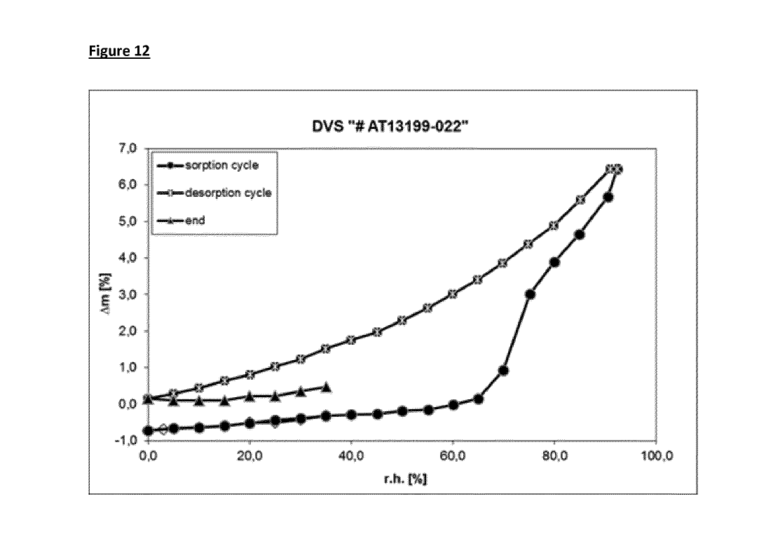

FIG. 13: illustrates a representative photomicrographic image of crystalline compound (I″a) (n-Propyl-Sofosbuvir) of the present invention under a polarizing light microscope.

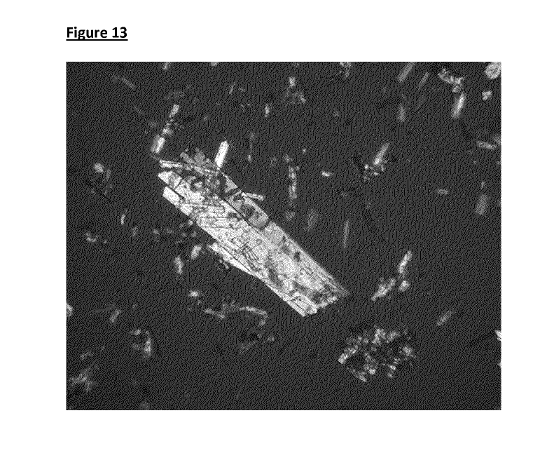

### DEFINITIONS

The term “sofosbuvir” as used herein refers to (S)-isopropyl 2-(((S)-(((2R,3R,4R,5R)-5-(2,4-dioxo-3,4-dihydropyrimidin-1(2H)-yl)-4-fluoro-3-hydroxy-4-methyltetrahydrofuran-2-yl)methoxy) (phenoxy)phosphoryl)-amino)propanoate according to formula (A) disclosed herein above.

The term “n-propyl-sofosbuvir” or “npropyl-sofosbuvir” or “n-Propyl-Sofosbuvir” as used herein refers to (S)-n-propyl 2-(((S)-(((2R,3R,4R,5R)-5-(2,4-dioxo-3,4-dihydropyrimidin-1(2H)-yl)-4-fluoro-3-hydroxy-4-methyltetrahydrofuran-2-yl)methoxy)(phenoxy)phosphoryl)-amino)propanoate according to formula (I″a) disclosed herein below.

The term “reflection” with regards to powder X-ray diffraction as used herein, means peaks in an X-ray diffractogram, which are caused at certain diffraction angles (Bragg angles) by constructive interference from X-rays scattered by parallel planes of atoms in solid material, which are distributed in an ordered and repetitive pattern in a long-range positional order. Such a solid material is classified as crystalline material, whereas amorphous material is defined as solid material, which lacks long-range order and only displays short-range order, thus resulting in broad scattering. According to literature, long-range order e.g. extends over approximately 100 to 1000 atoms, whereas short-range order is over a few atoms only (see “Fundamentals of Powder Diffraction and Structural Characterization of Materials” by Vitalij K. Pecharsky and Peter Y. Zayahj, Kluwer Academic Publishers, 2003, page 3).

As used herein, the term “amorphous” refers to a solid form of a compound that is not crystalline. An amorphous compound possesses no long-range order and does not display a definitive X-ray diffraction pattern with reflections.

With reference to powder X-ray diffraction, variabilities in reflection positions and relative intensities of the reflections are to be taken into account. For example, a typical precision of the 2-Theta values is in the range of ±0.2° 2-Theta, preferably in the range of ±0.1° 2-Theta. Thus, a reflection that usually appears at 7.6° 2-Theta for example can appear between 7.4° and 7.8° 2-Theta, preferably between 7.5 and 7.6° 2-Theta on most X-ray diffractometers under standard conditions. Furthermore, one skilled in the art will appreciate that relative reflection intensities will show inter-apparatus variability as well as variability due to degree of crystallinity, preferred orientation, sample preparation and other factors known to those skilled in the art and should be taken as qualitative measure only.

With reference to Fourier infrared spectrometry, variabilities in peak positions and relative intensities of the peaks are to be taken into account. For example, a typical precision of the wavenumber values is in the range of ±2 cm⁻¹. Thus, a peak at 1740 cm⁻¹ for example can appear in the range of from 1738 to 1742 cm⁻¹ on most infrared spectrometers under standard conditions. Differences in relative intensities are typically smaller compared to X-ray diffraction. However, one skilled in the art will appreciate that small differences in peak intensities due to degree of crystallinity, sample preparation and other factors can also occur in infrared spectroscopy. Relative peak intensities should therefore be taken as qualitative measure only.

The term “physical form” as used herein refers to any crystalline and/or amorphous phase of a compound.

A “predetermined amount” as used herein with regard to any of the compounds of the present invention refers to the initial amount of the respective compound used for the preparation of a pharmaceutical composition having a desired dosage strength.

The term “effective amount” as used herein with regard to any of the compounds of the present invention encompasses an amount of the respective compound which causes the desired therapeutic effect.

As used herein, the term “about” means within a statistically meaningful range of a value. Such a range can be within an order of magnitude, typically within 10%, more typically within 5%, even more typically within 1% and most typically within 0.1% of the indicated value or range. Sometimes, such a range can lie within the experimental error, typical of standard methods used for the measurement and/or determination of a given value or range.

### DETAILED DESCRIPTION OF THE INVENTION

In a first embodiment, the present invention relates to a compound of formula (I)

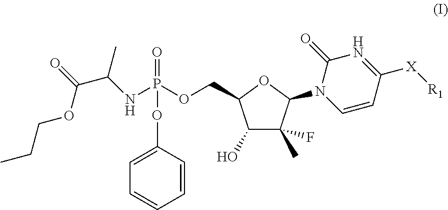

as well as isomers, stereoisomers, diastereoisomers and salts thereof, wherein X is O or NH and wherein when X is O R1 is H or a hydroxyl protecting group and when X is NH R1 is H or an amine protecting group. With regard to R1 when X is and R1 is a hydroxyl protecting group or when X is NH and R1 is an amine protecting group, no limitation exists as to the nature of R1 as long as it is capable of protecting a hydroxyl group or an amine group, respectively. Suitable protecting groups for hydroxyl and amine groups are commonly used in the art and known to the skilled person from, for example, T. W. Greene and G. M. Wuts, Protecting Groups in Organic Synthesis, Fourth Edition, Wiley, N.Y., 2007, or Fifth Edition, Wiley, N.Y., 2014. Preferably, in the compound of formula (I), X is O and R1 is hydrogen or a hydroxyl protecting group. Preferably, in the compound of formula (I), R1 is a hydroxyl protecting group selected from the group consisting of alkyl, silyl, benzyl and ester. Preferably, in the compound of formula (I), X is O and R1 is a silyl protecting group, preferably trimethylsilyl (TMS), triethylsilyl (TES), triisopropylsilyl (TIPS), dimethylisopropylsilyl (DMIPS), dimethylhexylsilyl (TDS), t-butyldimethylsilyl (TBS, TBDMS), t-butyldiphenylsilyl (TBDPS), triphenylsilyl (TPS), diphenylmethylsilyl (DPMS) or di-t-butylmethylsilyl (DTBMS). Preferably, in the compound of formula (I), X is O and R1 is an alkyl protecting group, more preferably ethyl. Preferably, in the compound of formula (I), X is O and R1 is a benzyl protecting group. Preferably, in the compound of formula (I), X is O and R1 is an ester protecting group, more preferably formate, acetate, benzoate, p-methoxybenzoate, benzoylformate, chloroacetate, dichloroacetate, trichloroacetate, trifluoroacetate, methoxyacetate, phenoxyacetate, p-chlorophenoxyacetate, phenylacetate, diphenylacetate, pivalate, benzoate and picolinate, even more preferably acetate, benzoate, pivalate or p-methoxybenzoate. Preferably, in the compound of formula (I), X is NH and R1 is hydrogen or an amine protecting group. Preferably, in the compound of formula (I), X is NH and R1 is an amine protecting group selected from the group consisting of benzyl, amide and carbamate. Preferably, in the compound of formula (I), X is NH and R1 is a benzyl protecting group. Preferably, in the compound of formula (I), X is NH and R1 (NH) is an amide protecting group, more preferably acetyl, chloroacetyl, benzoyl, formyl, trichloroacetyl, trifluoroacetyl, phenylacetyl, more preferably benzoyl. Preferably, in the compound of formula (I), X is NH and R1 is a carbamate protecting group, preferably methyl carbamate, ethyl carbamate, 9-fluorenylmethyl carbamate (Fmoc), t-butyl carbamate (Boc), allyl carbamate (Alloc) or vinyl carbamate (Voc).

In another aspect of the first embodiment, the present invention relates to a compound of formula (I), wherein the compound of formula (I) is the compound of formula (Ia) or the compound of formula (Ib)

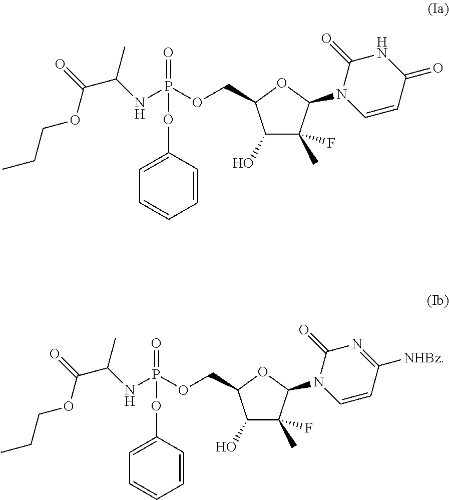

Throughout this invention and for all and any compounds, processes, compositions and any other examples contained herein, the term “Bz” denotes “benzoyl”, i.e. C6H5(CO)—. Preferably, the compound of formula (I) is the compound of formula (Ia)

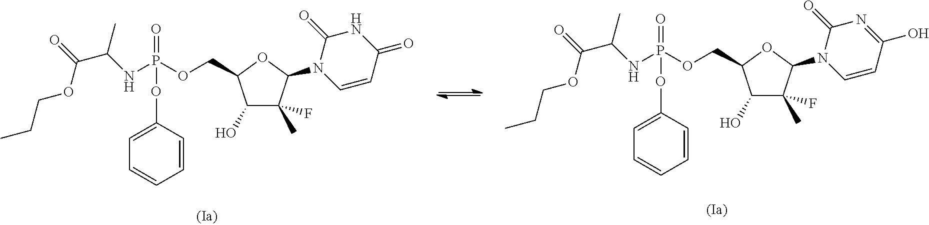 [SMILES: CCCOC(=O)C(C)NP(=O)(OC[C@H]1O[C@@H](n2ccc(=O)[nH]c2=O)[C@](C)(F)[C@@H]1O)Oc1ccccc1 ; CCCOC(=O)C(C)NP(=O)(OC[C@H]1O[C@@H](n2ccc(O)nc2=O)[C@](C)(F)[C@@H]1O)Oc1ccccc1]

More preferably, the compound of formula (I) is the compound of formula (I′)

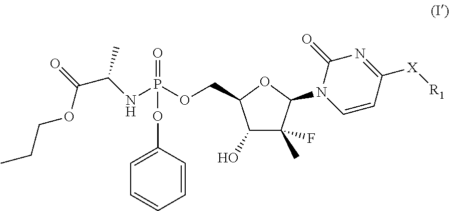

Preferably, the compound of formula (I′) is the compound of formula (I′a) or the compound of formula (I′b)

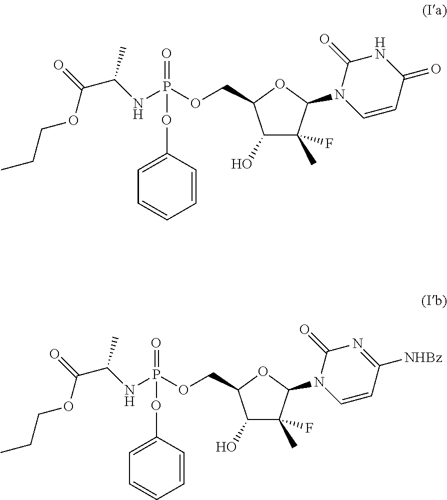

More preferably, the compound of formula (I′) is the compound of formula (I′a)

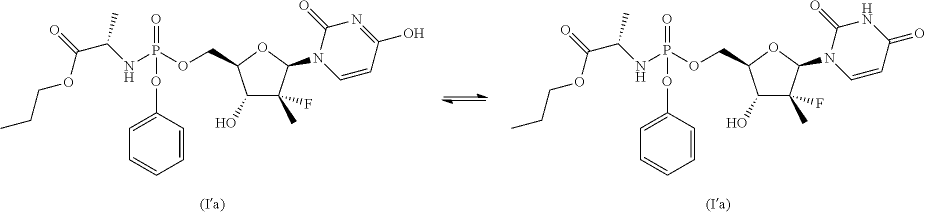 [SMILES: CCCOC(=O)[C@H](C)NP(=O)(OC[C@H]1O[C@@H](n2ccc(O)nc2=O)[C@](C)(F)[C@@H]1O)Oc1ccccc1 ; CCCOC(=O)[C@H](C)NP(=O)(OC[C@H]1O[C@@H](n2ccc(=O)[nH]c2=O)[C@](C)(F)[C@@H]1O)Oc1ccccc1]

Also preferably, the present invention relates to a compound of formula (I) wherein the compound of formula (I) is the compound of formula (I″) or the compound of formula (i″), in particular the compound of formula (I″)

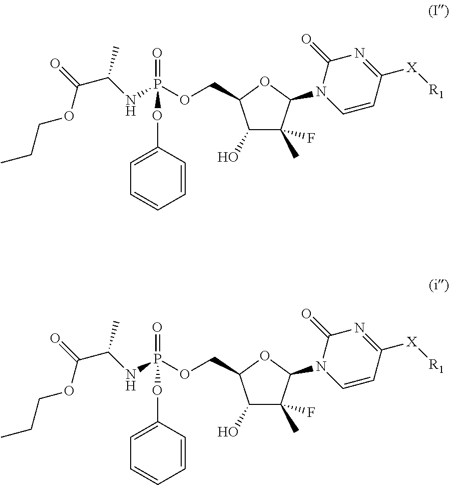

Preferably, the compound of formula (I″) is the compound of formula (I″a), the compound of formula (I″b), the compound of formula (i″a) or the compound of formula (i″b), more preferably the compound of formula (I″a) or the compound of formula (I″b)

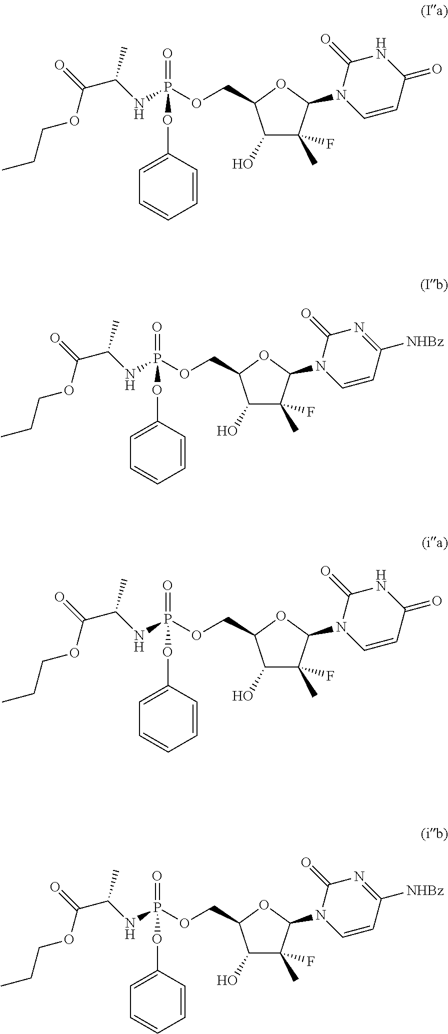

Also preferably, the compound of formula (I″) is the compound of formula (I″a) or the compound of formula (i″a), more preferably the compound of formula (I″a)

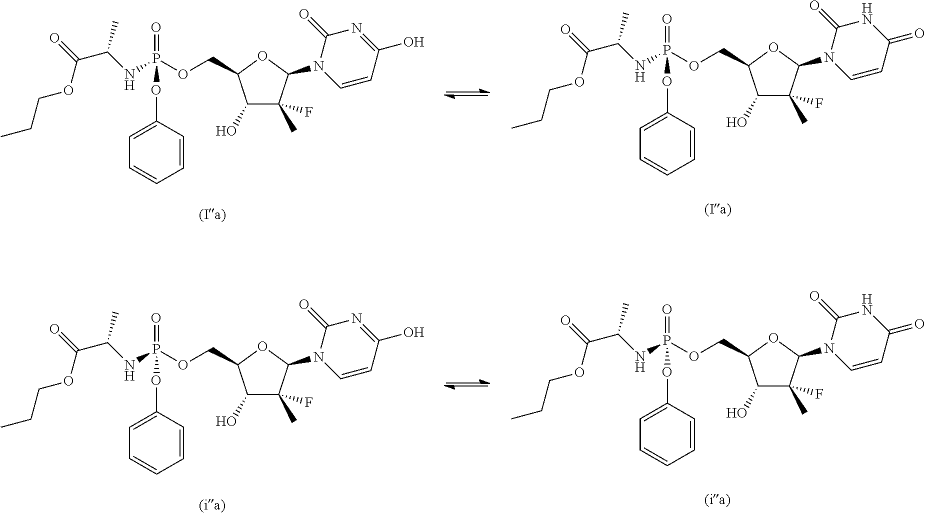 [SMILES: CCCOC(=O)[C@H](C)N[P@](=O)(OC[C@H]1O[C@@H](n2ccc(O)nc2=O)[C@](C)(F)[C@@H]1O)Oc1ccccc1 ; CCCOC(=O)[C@H](C)N[P@](=O)(OC[C@H]1O[C@@H](n2ccc(=O)[nH]c2=O)[C@](C)(F)[C@@H]1O)Oc1ccccc1 ; CCCOC(=O)[C@H](C)N[P@@](=O)(OC[C@H]1O[C@@H](n2ccc(O)nc2=O)[C@](C)(F)[C@@H]1O)Oc1ccccc1 ; CCCOC(=O)[C@H](C)N[P@@](=O)(OC[C@H]1O[C@@H](n2ccc(=O)[nH]c2=O)[C@](C)(F)[C@@H]1O)Oc1ccccc1]

Especially preferred is the compound of formula (I″a)

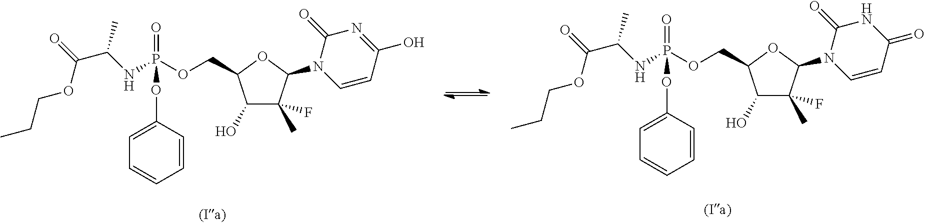 [SMILES: CCCOC(=O)[C@H](C)N[P@](=O)(OC[C@H]1O[C@@H](n2ccc(O)nc2=O)[C@](C)(F)[C@@H]1O)Oc1ccccc1 ; CCCOC(=O)[C@H](C)N[P@](=O)(OC[C@H]1O[C@@H](n2ccc(=O)[nH]c2=O)[C@](C)(F)[C@@H]1O)Oc1ccccc1]

Any of the compounds of the general formula (I) or of any of the preferred formulae described above can exist in amorphous form, one or more crystalline forms or mixtures of two or more thereof. Thus, the present invention relates to any of the compounds described above in amorphous, crystalline or pseudo-crystalline form or mixtures thereof. In particular, the present invention relates to any of the compounds described above in crystalline form.

A preferred compound is the compound of formula (I″a) in crystalline form. A crystalline form of the compound of formula (I″a) as described above is preferred having an X-ray powder diffraction pattern comprising reflections at 2-theta angles of (5.1±0.2)°, (6.9±0.2)°, (9.2±0.2)°, (16.3±0.2)°, (20.4±0.2)° when measured at a temperature in the range of from 15 to 25° C. with Cu-K_{alpha1,2} radiation having a wavelength of 0.15419 nm. Preferably, a crystalline form of the compound of formula (I″a) as described above comprises the above-described X-ray powder diffraction pattern as well as further reflections at 2-theta angles of (8.0±0.2)°, (15.3±0.2)°, (16.7±0.2)°, (17.9±0.2)°, (25.6±0.2)° when measured at a temperature in the range of from 15 to 25° C. with Cu-K_{alpha1,2} radiation having a wavelength of 0.15419 nm. A preferred crystalline form of the compound of formula (I″a) is that having a monoclinic space group symmetry and the following unit cell parameters as determined by an X-ray single crystal structure analysis at 173K:

a=12.8656 Angstrom

b=6.0028 Angstrom

c=17.5417 Angstrom

α=90°

β=98.397°

γ=90°

Also a preferred crystalline form of the compound of formula (I″a) is that having a melting point in the range of from 77.5° C. to 82.7° C. when measured via differential scanning calorimetry at a heating rate of 10K/min.

In a second embodiment, the present invention relates to processes for the preparation of any of the compounds described above. In particular, a first aspect of the present invention relates to a process for the preparation of a compound of formula (I) as described above comprising
(i) providing a compound of formula (II) or a mixture comprising the compound of formula (II)
(ii) reacting the compound of formula (II) with a compound of formula (III) to get a compound of formula (I)
(iii) optionally isolating the compound of formula (I)

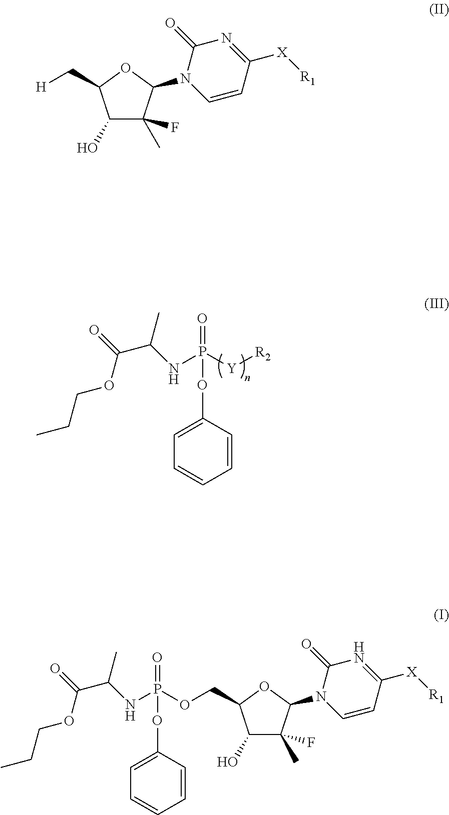

wherein (Y)ₙR₂ is a suitable leaving group for a nucleophilic substitution reaction. With regard to (Y)ₙR₂, no limitation exists as to the nature of (Y)ₙR₂ as long as it is capable of acting as a suitable leaving group in a nucleophilic substitution reaction. Suitable leaving groups in nucleophilic substitution reactions are commonly used in the art and known to the skilled person from, for example, T. W. Greene and G. M. Wuts, Protecting Groups in Organic Synthesis, Fourth Edition, Wiley, N.Y., 2007, or Fifth Edition, Wiley, N.Y., 2014.

Preferably, in the above-described process, n is 0 or 1 and Y is O, N or S. Preferably, in the above-described process , n is 1 and R₂ is alkyl, aryl, or heteroaryl, each optionally substituted with one or more electron-withdrawing groups, preferably aryl optionally substituted with one or more electron-withdrawing groups, more preferably phenyl optionally substituted with one or more electron-withdrawing groups. Preferably, in the above-described process, n is 1 and R₂ is phenyl substituted with one or more electron-withdrawing groups, wherein the one or more electron-withdrawing groups are preferably F, Cl, Br, I, or NO₂. Preferably, in the above-described process, n is 1, Y is O or S and R₂ is

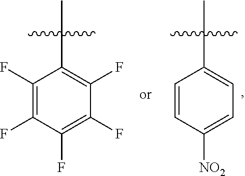 [SMILES: *c1c(F)c(F)c(F)c(F)c1F ; *c1ccc([N+](=O)[O-])cc1]

more preferably R₂ is

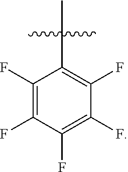 [SMILES: *c1c(F)c(F)c(F)c(F)c1F]

Preferably, in any of the above-described processes n is 1 and R₂ is a residue of formula (A)

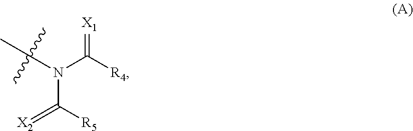

a residue of formula (B)

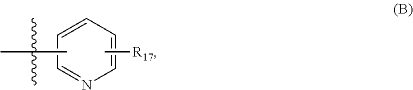

a residue of formula (C)

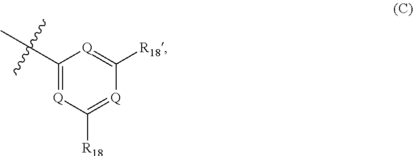

or a residue of formula (D)

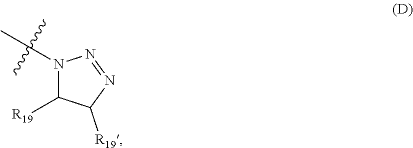

wherein at each occurrence

X₁ and X₂ are independently O or S;

R₄ and R₅ are independently H, OH, NH₂, C₁-C₆ alkyl or C₁-C₆ alkoxy, or

R₄ and R₅, together with the structure —C—N—C— according to formula (A), form an optionally substituted, 5-, 6-, or 7-membered saturated or partially unsaturated ring, wherein said ring is optionally fused to a 5- or 6-membered, optionally substituted ring which is a C₅-C₆ cycloalkyl, an aryl or a heterocycle comprising one or more heteroatoms independently being N, O or S;

R₁₇ is an electron-withdrawing group, preferably F, Cl, Br, I, NO₂, CHO, COOH, COO—(C₁-C₆)alkyl, CN, or COCl;

R₁₈ and R_{18′} are independently F, Cl, Br, I, or C₁-C₆ alkoxy;

each Q is independently C or N, wherein at least one Q is N;

R₁₉ and R_{19′} are independently H, OH, NH₂, C₁-C₆ alkyl optionally substituted with at least one of OH and NH₂, or C₁-C₆ alkoxy optionally substituted with at least one of OH and NH₂; or

R₁₉ and R_{19′} taken together form an optionally substituted 5-, 6-, or 7-membered saturated or partially unsaturated or aromatic ring, wherein the ring is optionally fused to a 5- or 6-membered, optionally substituted ring which is a C₅-C₆ cycloalkyl, an aryl, preferably benzo, or a heterocycle comprising one or more heteroatoms independently being N, O or S, the 5- or 6-membered optionally substituted ring preferably being heteroaryl.

Preferably, in any of the above-described processes n is 0 and R₂ is a residue of formula (A1)

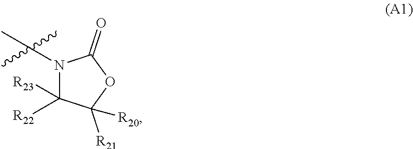

wherein R₂₀, R₂₁, R₂₂ and R₂₃ are each independently H, aryl, or C₁-C₆ alkyl optionally substituted with at least one of C₁-C₆ alkoxy optionally substituted with at least one of OH and NH₂; or

R₂₀ and R₂₂, or R₂₀ and R₂₃, or R₂₁ and R₂₂, or R₂₁ and R₂₃ when taken together form an optionally substituted 5-, 6-, or 7-membered saturated or partially unsaturated or aromatic ring which is an aryl, preferably
benzo, or a heterocycle comprising one or more heteroatoms independently being N, O or S, the 5-, 6-, or 7-membered saturated or partially unsaturated or aromatic ring preferably being heteroaryl. Regarding the 5-, 6-, or 7-membered saturated or partially unsaturated or aromatic ring in any of the processes and/or leaving groups described above, no limitation exists as long as nucleophilic substitution reaction leading to a compound of formula (I) takes place. Preferably, in the above-described process the substituent of the optionally substituted 5-, 6-, or 7-membered saturated or partially unsaturated or aromatic ring which is an aryl, preferably benzo, or a heterocycle comprising one or more heteroatoms independently being N, O or S, is at least a substituent, preferably one substituent, selected from the group consisting of OH, C1-C6 alkoxy, aryl, heteroaryl, C3-C6 cycloalkyl, F, Cl, Br, I, COOH, CHO, C(O)(C1-C6 alkyl), C(O)(aryl), COO(C1-C6 alkyl), COONH2, COONH(C1-C6 alkyl), CN, NO2, —NH2, NR27R28, wherein R27 and R28 are independently selected from the group consisting of H, C1-C6 alkyl, C1-C6 alkoxy, aryl, heteroaryl, and wherein aryl at each occurrence is preferably phenyl.

Preferably, in the above-described process the aromatic ring is a benzo substituted with at least one, preferably with one substituent, wherein the substituent is selected from the group consisting of OH, C1-C6 alkoxy, aryl, heteroaryl, C3-C6 cycloalkyl, F, Cl, Br, I, COOH, CHO, C(O)(C1-C6 alkyl), C(O)(aryl), COO(C1-C6 alkyl), COONH2, COONH(C1-C6 alkyl), CN, NO2, —NH2, NR27R28, wherein R27 and R28 are independently selected from the group consisting of H, C1-C6 alkyl, C1-C6 alkoxy, aryl, heteroaryl, and wherein aryl at each occurrence is preferably phenyl. Preferably, R₂₂ and R₂₃ are each independently H, aryl, or C1-C6 alkyl substituted with at least one of C1-C6 alkoxy optionally substituted with at least one of OH and NH2.

Preferably, in any of the above-described processes n is 1 and R₂ is a residue of formula (A)

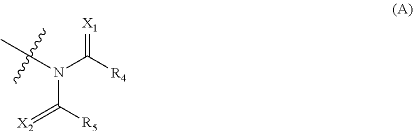

wherein

X₁ and X₂ are independently O or S;

R₄ and R₅ are independently H, OH, NH₂, C₁-C₆ alkyl or C₁-C₆ alkoxy, or

R₄ and R₅, together with the structure —C—N—C— according to formula (A), form an optionally substituted, 5-, 6-, or 7-membered saturated or partially unsaturated ring, wherein said ring is optionally fused to a 5- or 6-membered, optionally substituted ring which is a C₅-C₆ cycloalkyl, an aryl or a heterocycle comprising one or more heteroatoms independently being N, O or S.

More preferably, R₂ is a residue of formula (IIb)

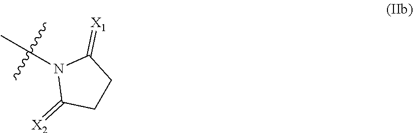

More preferably, R₂ is a residue of formula (IIc)

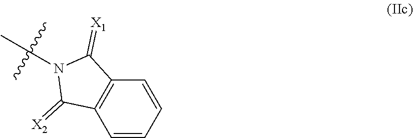

More preferably, X1 is O and X2 is O.

Preferably, in any of the above-described processes n is 1 and R₂ is a residue of formula (B)

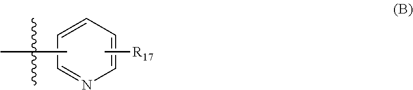

Preferably, R17 is selected from the group consisting of F, Cl, Br, I, NO2, CHO, COOH, COO—(C1-C6)alkyl, CN and COCl.

Preferably, in any of the above-described processes n is 1 and R₂ is a residue of formula (C)

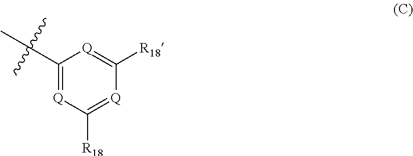

Preferably, R18 and R18′ are independently F, Cl, Br, I, or C1-C6 alkoxy and each Q is independently C or N, wherein at least one Q is N.

Preferably, in any of the above-described processes n is 1 and R₂ is a residue of formula (D)

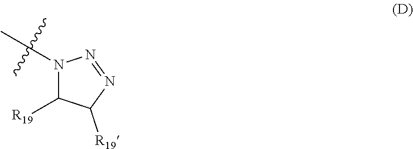

wherein R₁₉ and R_{19′} are independently H, OH, NH₂, C₁-C₆ alkyl optionally substituted with at least one of OH and NH₂, or C₁-C₆ alkoxy optionally substituted with at least one of OH and NH₂; or R₁₉ and R_{19′} taken together form an optionally substituted 5-, 6-, or 7-membered saturated or partially unsaturated or aromatic ring, wherein the aromatic ring is preferably benzo, wherein the ring is optionally fused to a 5-or 6-membered, optionally substituted ring which is a C₅-C₆ cycloalkyl, an aryl, preferably benzo, or a heterocycle comprising one or more heteroatoms independently being N, O or S, the 5- or 6-membered optionally substituted ring preferably being heteroaryl. Preferably, the substituent of the optionally substituted 5-, 6-, or 7-membered saturated or partially unsaturated or aromatic ring is at least a substituent, preferably one substituent, selected from the group consisting of OH, C1-C6 alkoxy, aryl, heteroaryl, C3-C6 cycloal-kyl, F, Cl, Br, I, COOH, CHO, C(O)(C1-C6 alkyl), C(O)(aryl), COO(C1-C6 alkyl), COONH2, COONH(C1-C6 alkyl), CN, NO2, —NH2, NR27R28, wherein R27 and R28 are independently selected from the group consisting of H, C1-C6 alkyl, C1-C6 alkoxy, aryl, heteroaryl, and wherein aryl at each occurrence is preferably phenyl. Preferably, the aromatic ring formed by R19 and R19′ taken together is a benzo substituted with at least one, preferably with one substituent, wherein the substituent is selected from the group consisting of OH, C1-C6 alkoxy, aryl, heteroaryl, C3-C6 cycloalkyl, F, Cl, Br, I, COOH, CHO, C(O)(C1-C6 alkyl), C(O)(aryl), COO(C1-C6 alkyl), COONH2, COONH(C1-C6 alkyl), CN, NO2, —NH2, NR27R28, wherein R27 and R28 are independent-ly selected from the group consisting of H, C1-C6 alkyl, C1-C6 alkoxy, aryl, heteroaryl, and wherein aryl at each occurrence is preferably phenyl.

Preferably, in any of the above-described processes n is 1, Y is O and R2 is

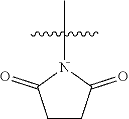 [SMILES: *N1C(=O)CCC1=O]

Preferably, in any of the above-described processes n is 0 and R2 is Cl.

Preferably, in any of the above-described processes X is O and R1 is hydrogen.

Preferably, in any of the above-described processes X is NH and R1 is hydrogen.

Preferably, in any of the above-described processes X is O and R1 is a hydroxyl protecting group, preferably a hydroxyl protecting group selected from the group consisting of alkyl, silyl, benzyl and ester. Preferably, X is O and R1 is a silyl protecting group, preferably trimethylsilyl (TMS), triethylsilyl (TES), triisopropylsilyl (TIPS), dimethylisopropylsilyl (DMIPS), dimethylhexylsilyl (TDS), t-butyldimethylsilyl (TBS, TBDMS), t-butyldiphenylsilyl (TBDPS), triphenylsilyl (TPS), diphenylmethylsilyl (DPMS) or di-t-butylmethylsilyl (DTBMS).

Preferably, in any of the above-described processes X is O and R1 is an alkyl protecting group, preferably ethyl.

Preferably, in any of the above-described processes X is O and R1 is a benzyl protecting group.

Preferably, in any of the above-described processes X is O and R1 is an ester protecting group, preferably formate, acetate, benzoate, p-methoxybenzoate, benzoylformate, chloroacetate, dichloroacetate, trichloroacetate, trifluoroacetate, methoxyacetate, phenoxyacetate, p-chlorophenoxyacetate, phenylacetate, diphenylacetate, pivalate, benzoate and picolinate, more preferably acetate, benzoate, pivalate or p-methoxybenzoate

Preferably, in any of the above-described processes X is NH and R1 is an amine protecting group preferably selected from the group consisting of benzyl, amide and carbamate.

Preferably, in any of the above-described processes X is NH and R1 is a benzyl protecting group.

Preferably, in any of the above-described processes X is NH and R1 is an amide protecting group, preferably acetyl, chloroacetyl, benzoyl, formyl, trichloroacetyl, trifluoroacetyl, phenylacetyl, more preferably benzoyl.

Preferably, in any of the above-described processes X is NH and R1 is a carbamate protecting group, preferably methyl carbamate, ethyl carbamate, 9-fluorenylmethyl carbamate (Fmoc), t-butyl carbamate (Boc), allyl carbamate (Alloc), vinyl carbamate (Voc).

With regard to R1, which can be a hydroxyl protecting group, an alkyl protecting group, a benzyl protecting group, an ester protecting group, an amine protecting group, an amide protecting group or a carbamate protecting group depending on the nature of X, no limitation exists as to the nature of R1 as long as it is capable of acting as a hydroxyl protecting group, an alkyl protecting group, a benzyl protecting group, an ester protecting group, an amine protecting group, an amide protecting group or a carbamate protecting group, respectively. Suitable protecting groups as described above are commonly used in the art and known to the skilled person from, for example, T. W. Greene and G. M. Wuts, Protecting Groups in Organic Synthesis, Fourth Edition, Wiley, N.Y., 2007, or Fifth Edition, Wiley, N.Y., 2014.

While any of the above-described compounds of formula (I) can be prepared by the processes also described above, it is preferred that the compound of formula (I) prepared by any of the above-described processes is the compound of formula (Ia) or (Ib)

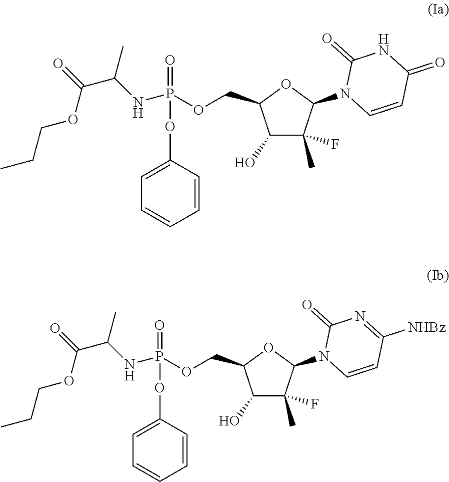

Preferably, the compound of formula (I) is the compound of formula (Ia)

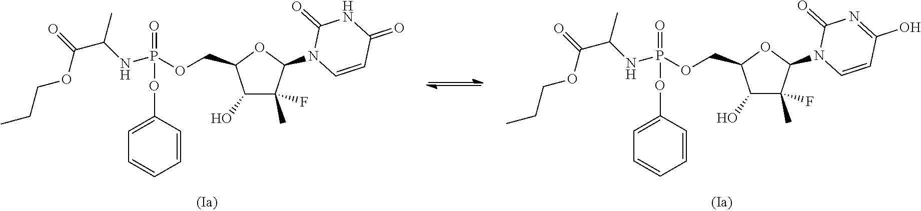 [SMILES: CCCOC(=O)C(C)NP(=O)(OC[C@H]1O[C@@H](n2ccc(=O)[nH]c2=O)[C@](C)(F)[C@@H]1O)Oc1ccccc1 ; CCCOC(=O)C(C)NP(=O)(OC[C@H]1O[C@@H](n2ccc(O)nc2=O)[C@](C)(F)[C@@H]1O)Oc1ccccc1]

Preferably, the compound of formula (I) is the compound of formula (I′) and the compound of formula (III) is the compound of formula (III′)

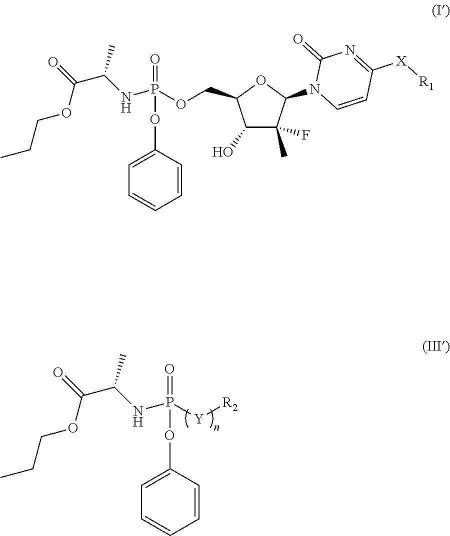

Preferably, the compound of formula (I′) is the compound of formula (I′a) or (I′b)

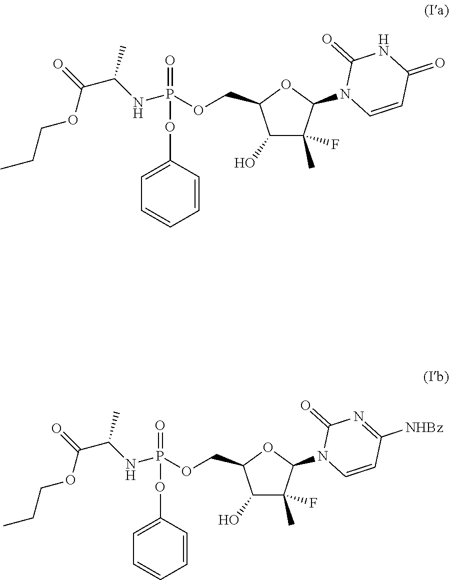

Preferably, the compound of formula (I′) is the compound of formula (I′a)

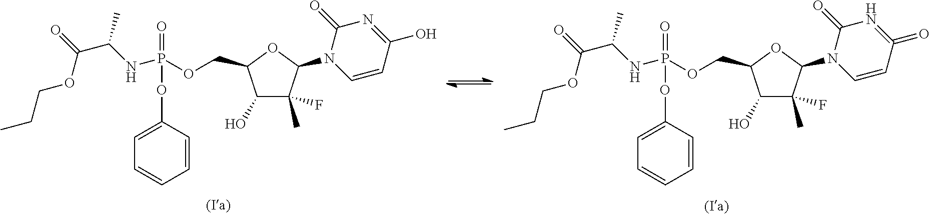

Preferably, the compound of formula (I) is the compound of formula (I″) or the compound of formula (I″) and the compound of formula (III) is the compound of formula (III″) or the compound of formula (iii″)

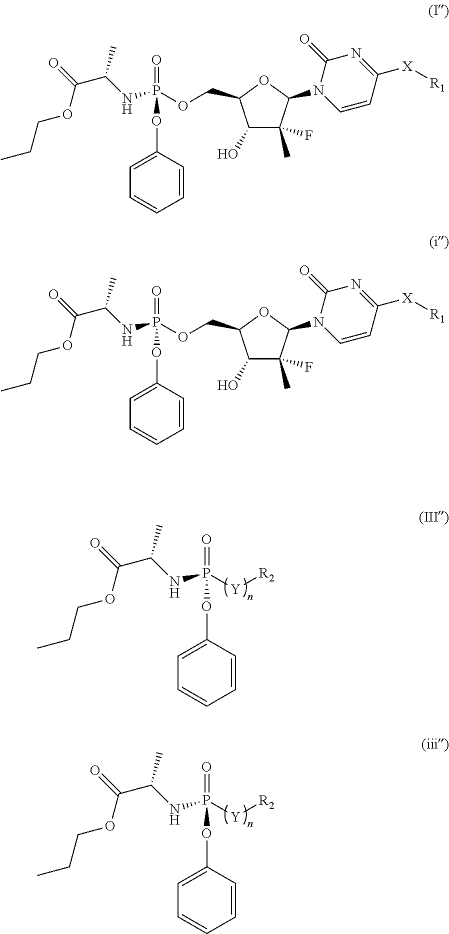

Preferably, the compound of formula (I″) is the compound of formula (I″a), the compound of formula (I″b), the compound of formula (i″a) or the compound of formula (i″b), more preferably the compound of formula (I″a) or the compound of formula (I″b)

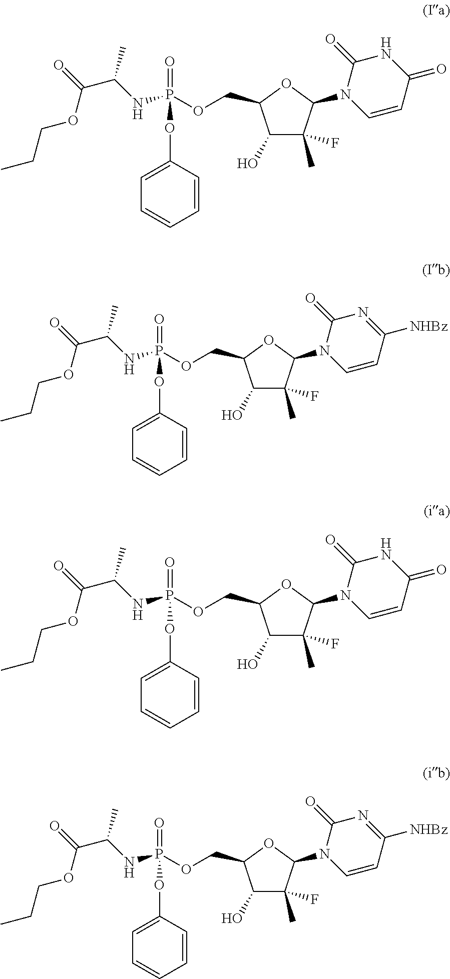

Preferably the compound of formula (I″) is the compound of formula (I″a) or the compound of formula (i″a), more preferably the compound of formula (I″a)

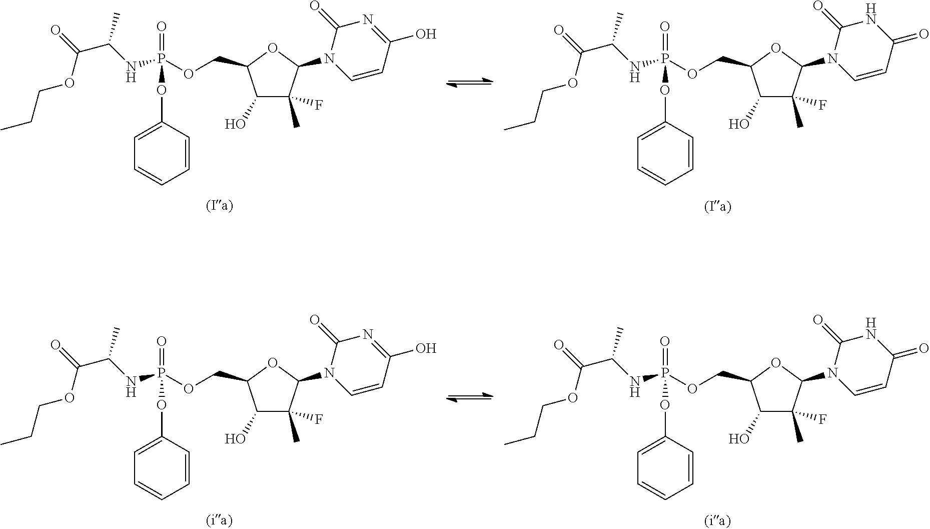 [SMILES: CCCOC(=O)[C@H](C)N[P@](=O)(OC[C@H]1O[C@@H](n2ccc(O)nc2=O)[C@](C)(F)[C@@H]1O)Oc1ccccc1 ; CCCOC(=O)[C@H](C)N[P@](=O)(OC[C@H]1O[C@@H](n2ccc(=O)[nH]c2=O)[C@](C)(F)[C@@H]1O)Oc1ccccc1 ; CCCOC(=O)[C@H](C)N[P@@](=O)(OC[C@H]1O[C@@H](n2ccc(O)nc2=O)[C@](C)(F)[C@@H]1O)Oc1ccccc1 ; CCCOC(=O)[C@H](C)N[P@@](=O)(OC[C@H]1O[C@@H](n2ccc(=O)[nH]c2=O)[C@](C)(F)[C@@H]1O)Oc1ccccc1]

Regarding the reaction conditions for the preparation of any of the above-described compounds by any of the above-described processes, no limitation exists as long as the desired compound of formula (I) is obtained. Preferably, step (ii) is carried out in the presence of one or more bases. Preferably, the one or more bases are organic bases. Preferably, the one or more bases comprise an alkylmagnesium halide. Preferably, the alkylmagnesium halide is tert-butylmagnesium chloride. Preferably, the one or more bases are selected from the group consisting of an amine, an amidine, a heteroaromatic compound comprising a basic ring-nitrogen atom, and a mixture of two or more thereof, more preferably selected from the group consisting of ethyldiisopropylamine, triethylamine, diethylamine, 1,8-diazabicycloundec-7-ene, pyridine, quinoline, isoquinoline, acridine, pyrazine, imidazole, benzimidazole, pyrazole, and a mixture of two or more thereof.

Preferably, prior to the reaction according to (ii), the molar ratio of the one or more bases relative to the compound of formula (III) is in the range of from 0.1:1 to 5:1 wherein, if more than one base is comprised in the mixture provided in a), the molar ratio relates to the total molar amount of all bases. Preferably, the molar ratio of the one or more bases relative to the compound of formula (III) is in the range of from 0.1:1 to 2:1 preferably in the range of from 0.5:1 to 1.2:1 wherein, if more than one base is comprised in the mixture provided in a), the molar ratio relates to the total molar amount of all bases.

Preferably, the mixture provided in (i) further comprises one or more solvents and one or more bases, wherein prior to the reaction according to (ii), the molar ratio of the one or more bases relative to the compound of formula (III) is in the range of from 0.1:1 to 5:1.

Regarding any further components present in the reaction mixture for the preparation of any of the above-described compounds by any of the above-described processes, no limitation exists as long as the desired compound of formula (I) is obtained. It has been found that the presence of one or more Lewis acids is advantageous to the reaction. Thus preferably, step (ii) is carried out in the presence of one or more Lewis acids.

Preferably, the one or more Lewis acids comprise a twice positively charged ion or a three times positively charged ion.

Preferably, the one or more Lewis acids comprise a twice positively charged metal ion or a three times positively charged metal ion.

Preferably, the twice positively charged ion is a Zn ion, a Mg ion, a Cu ion, or an Fe ion.

Preferably, the twice positively charged ion is a Zn ion.

Preferably, the one or more Lewis acids is one or more of ZnBr2, ZnCl2, ZnI2.

The process of any of embodiments 81 to 86, wherein the one or more Lewis acids comprises, preferably is ZnBr2.

Preferably, the one or more Lewis acids is one or more of ZnBr2, ZnCl2, ZnI2, MgBr2, MgBr2•OEt2, CuCl2, Cu(acetylacetonate)2, and Fe(II) fumarate.

Preferably, the three times positively charged ion is a Mn ion.

Preferably, the one or more Lewis acids is Mn(acetylacetonate)3.

Regarding the solvent, solvents or solvent mixture for the reaction mixture for the preparation of any of the above-described compounds by any of the above-described processes, no limitation exists as long as the desired compound of formula (I) is obtained. Preferably, step (ii) is carried out in a suitable solvent or suitable solvent mixture.

Preferably, the suitable solvent or solvent mixture consists of or comprises a solvent selected from the list consisting of methylene chloride, methyl tert-butyl ether, tetrahydrofurane, dimethylsulphoxide, dimethylformamide, and a mixture of two or more thereof.

Preferably, prior to the reaction according to (ii), the molar ratio of the compound of formula (II) relative to the compound of formula (III) is in the range of from 0.5:1 to 5:1.

Preferably, the molar ratio of the compound of formula (II) relative to the compound of formula (III) is in the range of from 0.8:1 to 2:1, preferably in the range of from 0.9:1 to 1.2:1.

Preferably, prior to the reaction according to (ii), the molar ratio of the Lewis acid relative to the compound of formula (III) is in the range of from 0.1:1 to 5:1.

Preferably, the molar ratio of the Lewis acid relative to the compound of formula (III) is in the range of from 0.2:1 to 2:1, preferably in the range of from 0.5:1 to 1.2:1.

Regarding the temperature for the reaction for the preparation of any of the above-described compounds by any of the above-described processes, no limitation exists as long as the desired compound of formula (I) is obtained. Preferably, step (ii) is carried out at a temperature in the range of from 0 to 80° C.

Preferably, the temperature is in the range of from 10 to 65° C.

Preferably, the temperature is in the range of from 20 to 50° C.

Preferably, the reaction in step (ii) s carried out for a period of time in the range of from 0.5 to 48 h.

Preferably, the period of time is in the range of from 1 to 36 h.

Preferably, the period of time is in the range of from 2 to 24 h.

Preferably, the reaction conditions in step (ii) comprise a temperature of the mixture in the range of from 20 to 50° C., wherein according to (ii), the mixture is subjected to the reaction conditions for a period of time in the range of from 2 to 24 h.

Preferably, prior to the reaction according to (ii), the molar ratio of the compound of formula (II) relative to the compound of formula (III) is in the range of from 0.9:1 to 1.2:1, the molar ratio of the Lewis acid relative to the compound of formula (III) is in the range of from 0.5:1 to 1.2:1, and the molar ratio of the one or more bases relative to the compound of formula (III) is in the range of from 0.5:1 to 1.2:1 wherein, if more than one base is comprised in the mixture provided in a), the molar ratio relates to the total molar amount of all bases.

When X is O and R1 is a hydroxyl protecting group or when X is NH and R1 is an amine protecting group it might be useful to remove said protecting groups. Thus preferably, when X is O and R1 is a hydroxyl protecting group or when X is NH and R1 is an amine protecting group the process described above further comprises, after step (ii) or after optional step (iii),
(iv) removing the hydroxyl or amine protecting group to get a compound of formula (Ia), a compound of formula (I′a), a compound of formula (I″a) or a compound of formula (i″a)
(v) optionally isolating the compound of formula (Ia), the compound of formula (I′a), the compound of formula (I″a) or the compound of formula (i″a).

Regarding the removal of the hydroxyl or amine protecting groups and the experimental conditions required, no limitation exists as long as the desired compound is obtained. The removal of protecting groups, in particular of hydroxyl and/or amine protecting groups and more particularly the hydroxyl and/or amine protecting groups of the present invention, is known in the art and common practice for the skilled person as described, for example, in T. W. Greene and G. M. Wuts, Protecting Groups in Organic Synthesis, Fourth Edition, Wiley, N.Y., 2007, or Fifth Edition, Wiley, N.Y., 2014.

Preferably, X is O and R1 is benzyl and wherein removing the protecting group in (iv) comprises subjecting the protected compound to hydrogenolysis.

Preferably, X is O and R1 is an ester protecting group, preferably benzoyl and wherein removing the protecting group in (iv) comprises subjecting the protected compound to acidic, basic or reducing conditions, preferably basic or reducing conditions, preferably reducing conditions in the presence of LiAlH4.

Preferably, X is O and R1 is a silyl protecting group and wherein removing the protecting group in (iv) comprises subjecting the protected compound to acidic conditions.

Preferably, X is O and R1 is an alkyl protecting group, preferably ethyl and wherein removing the protecting group in (iv) comprises subjecting the protected compound to methanolic ammonia.

Preferably, X is NH and R1 is an amide protecting group, preferably benzoyl and wherein removing the protecting group in (iv) comprises subjecting the protected compound to acidic, basic or reducing conditions, preferably basic or reducing conditions, preferably reducing conditions in the presence of LiAlH4.

Preferably, X is NH and R1 is benzyl and wherein removing the protecting group in (iv) comprises subjecting the protected compound to hydrogenolysis.

Thus preferably, the compound of formula (Ia), preferably the compound of formula (I′a), more preferably the compound of formula (I″a), is obtained after step (iv) or after optional step (v).

Regarding the isolation of the desired compound of formula (I) in step (iii) or step (v), no limitation exists as long as the desired compound is obtained. Thus preferably, isolating in step (iii) or step (v) is achieved by, consists of or comprises precipitation, crystallization or chromatography.

Preferably, crystallization comprises seeding.

Preferably, crystallization comprises using a solvent mixture comprising dichloromethane and heptane.

Preferably, the dichloromethane and heptane are used in a volume ratio of from 30:30 to 60:10, preferably of from 70:20 to 30:20, preferably of from 45:25 to 55:15.

Preferably, crystallization is carried out at a temperature of from 0 to 40° C., preferably of from 20 to 30° C. In a particularly preferred aspect, the present invention relates to a process for the preparation of a compound of formula (I″a) in crystalline form comprising
(i) providing a solution of the compound of formula (I″a) in a suitable solvent or solvent mixture,
(ii) subjecting the solution of (i) to crystallization conditions
(iii) isolating the crystalline compound of formula (I″a)

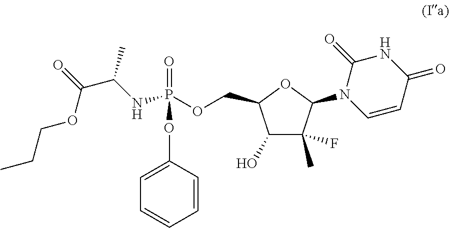 [SMILES: CCCOC(=O)[C@H](C)N[P@](=O)(OC[C@H]1O[C@@H](n2ccc(=O)[nH]c2=O)[C@](C)(F)[C@@H]1O)Oc1ccccc1]

Preferably, the solvent or solvent mixture in step (i) above comprises one or more solvents selected from dichloromethane and ethyl acetate, preferably dichloromethane, or mixtures thereof. Preferably, the solvent or solvent mixture in (i) comprises dichloromethane, preferably wherein the solvent in (i) is dichloromethane. Preferably, providing a solution of the compound of formula (I″a) in a suitable solvent or solvent mixture in (i) comprises treating the compound of formula (I″a) in the solvent or solvent mixture with activated charcoal and/or silica gel, preferably with activated charcoal and silica gel and filtering the resulting mixture to obtain a clear solution. Preferably, subjecting the solution of (i) to crystallization conditions in (ii) comprises adding a further solvent or solvent mixture. Preferably, the further solvent or solvent mixture consists of or comprises pentane, hexane, heptane, diisopropyl ether, preferably heptane, or mixtures thereof. Preferably, the further solvent or solvent mixture comprises heptane, preferably wherein the further solvent in (ii) is heptane. Preferably, the further solvent or solvent mixture is added in a volume ratio of from 30:30 to 10:60, preferably of from 20:70 to 20:30, preferably of from 25:45 to 55:55 relative to the volume of the solvent or solvent mixture provided in (i). Preferably, step (ii) comprises storing the mixture for a period of time in the range of from 1 to 72 hours, preferably of from 1 to 17 hours. Preferably, step (ii) comprises storing the mixture at a temperature in the range of from 0 to 40° C., preferably in the range of from 20 to 30° C. Preferably, step (ii) comprises storing the mixture for a period of time in the range of from 1 to 72 hours, preferably of from 1 to 17 hours at a temperature in the range of from 0 to 40° C., preferably in the range of from 20 to 30° C. Preferably, step (ii) comprises seeding. Preferably, step (iii) comprises filtering, preferably filtering under vacuum, the resulting crystalline solid. Preferably, step (iii) comprises drying the resulting crystalline solid. Preferably, step (iii) comprises drying the resulting crystalline solid at a temperature of from 15 to 60° C., preferably of from 15 to 40° C., preferably of from 20 to 30° C., preferably of from 20 to 25° C., more preferably at 23° C. and at a pressure of from 5 to 100 mbar, preferably of from 15 to 80 mbar, preferably of from 20 to 50 mbar, more preferably of 30 mbar.

In a third embodiment, the present invention relates to compounds and intermediates present, resulting from or involved in any of the above-described processes. Thus, the present invention relates to a compound of formula (III)

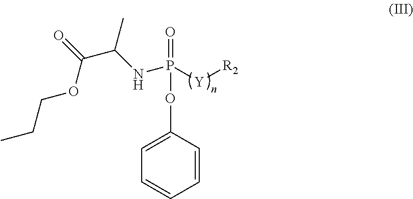

wherein (Y)ₙR₂ is a suitable leaving group for a nucleophilic substitution reaction. With regard to (Y)ₙR₂, no limitation exists as to the nature of (Y)ₙR₂ as long as it is capable of acting as a suitable leaving group in a nucleophilic substitution reaction. Suitable leaving groups in nucleophilic substitution reactions are commonly used in the art and known to the skilled person from, for example, T. W. Greene and G. M. Wuts, Protecting Groups in Organic Synthesis, Fourth Edition, Wiley, N.Y., 2007, or Fifth Edition, Wiley, N.Y., 2014. Thus preferably, n is 0 or 1 and wherein Y is O, N or S.

Preferably, n is 1 and R₂ is alkyl, aryl, or heteroaryl, each optionally substituted with one or more electron-withdrawing groups, preferably aryl optionally substituted with one or more electron-withdrawing groups, more preferably phenyl optionally substituted with one or more electron-withdrawing groups.

Preferably, n is 1 and R₂ is phenyl substituted with one or more electron-withdrawing groups, wherein the one or more electron-withdrawing groups are preferably F, Cl, Br, I, or NO₂.

Preferably, n is 1, Y is O or S and R₂ is

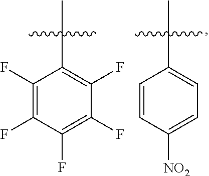

more preferably R₂ is

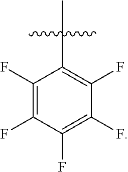 [SMILES: *c1c(F)c(F)c(F)c(F)c1F]

Preferably, n is 1 and R₂ is a residue of formula (A)

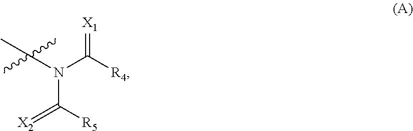

a residue of formula (B)

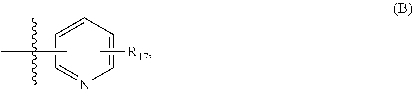

a residue of formula (C)

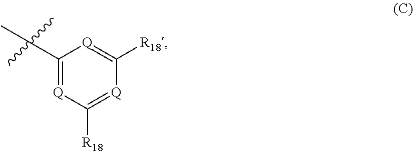

or a residue of formula (D)

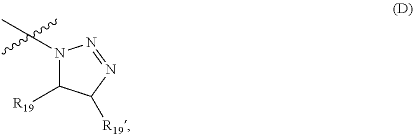

wherein at each occurrence

X₁ and X₂ are independently O or S;

R₄ and R₅ are independently H, OH, NH₂, C₁-C₆ alkyl or C₁-C₆ alkoxy, or

R₄ and R₅, together with the structure —C—N—C— according to formula (A), form an optionally substituted, 5-, 6-, or 7-membered saturated or partially unsaturated ring, wherein said ring is optionally fused to a 5- or 6-membered, optionally substituted ring which is a C₅-C₆ cycloalkyl, an aryl or a heterocycle comprising one or more heteroatoms independently being N, O or S;

R₁₇ is an electron-withdrawing group, preferably F, Cl, Br, I, NO₂, CHO, COOH, COO—(C₁-C₆)alkyl, CN, or COCl;

R₁₈ and R_{18′} are independently F, Cl, Br, I, or C₁-C₆ alkoxy;

each Q is independently C or N, wherein at least one Q is N;

R₁₉ and R_{19′} are independently H, OH, NH₂, C₁-C₆ alkyl optionally substituted with at least one of OH and NH₂, or C₁-C₆ alkoxy optionally substituted with at least one of OH and NH₂; or

R₁₉ and R_{19′} taken together form an optionally substituted 5-, 6-, or 7-membered saturated or partially unsaturated or aromatic ring, wherein the ring is optionally fused to a 5- or 6-membered, optionally substituted ring which is a C₅-C₆ cycloalkyl, an aryl, preferably benzo, or a heterocycle comprising one or more heteroatoms independently being N, O or S, the 5- or 6-membered optionally substituted ring preferably being heteroaryl.

Preferably, n is 0 and R₂ is a residue of formula (A1)

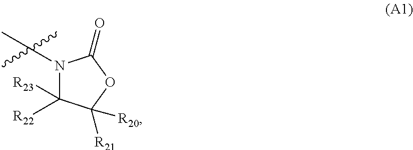

wherein R₂₀, R₂₁, R₂₂ and R₂₃ are each independently H, aryl, or C₁-C₆ alkyl optionally substituted with at least one of C₁-C₆ alkoxy optionally substituted with at least one of OH and NH₂; or

R₂₀ and R₂₂, or R₂₀ and R₂₃, or R₂₁ and R₂₂, or R₂₁ and R₂₃ when taken together form an optionally substituted 5-, 6-, or 7-membered saturated or partially unsaturated or aromatic ring which is an aryl, preferably benzo, or a heterocycle comprising one or more heteroatoms independently being N, O or S, the 5-, 6-, or 7-membered saturated or partially unsaturated or aromatic ring preferably being heteroaryl.

Preferably, the substituent of the optionally substituted 5-, 6-, or 7-membered saturated or partially unsaturated or aromatic ring which is an aryl, preferably benzo, or a heterocycle comprising one or more heteroatoms independently being N, O or S, is at least a substituent, preferably one substituent, selected from the group consisting of OH, C1-C6 alkoxy, aryl, heteroaryl, C3-C6 cycloalkyl, F, Cl, Br, I, COOH, CHO, C(O)(C1-C6 alkyl), C(O)(aryl), COO(C1-C6 alkyl), COONH2, COONH(C1-C6 alkyl), CN, NO2, —NH2, NR27R28, wherein R27 and R28 are independently selected from the group consisting of H, C1-C6 alkyl, C1-C6 alkoxy, aryl, heteroaryl, and wherein aryl at each occurrence is preferably phenyl.

Preferably, the aromatic ring is a benzo substituted with at least one, preferably with one substituent, wherein the substituent is selected from the group consisting of OH, C1-C6 alkoxy, aryl, heteroaryl, C3-C6 cycloalkyl, F, Cl, Br, I, COOH, CHO, C(O)(C1-C6 alkyl), C(O)(aryl), COO(C1-C6 alkyl), COONH2, COONH(C1-C6 alkyl), CN, NO2, —NH2, NR27R28, wherein R27 and R28 are independently selected from the group consisting of H, C1-C6 alkyl, C1-C6 alkoxy, aryl, heteroaryl, and wherein aryl at each occurrence is preferably phenyl.

Preferably, R22 and R23 are each independently H, aryl, or C1-C6 alkyl substituted with at least one of C1-C6 alkoxy optionally substituted with at least one of OH and NH2.

Preferably, n is 1 and R₂ is a residue of formula (A)

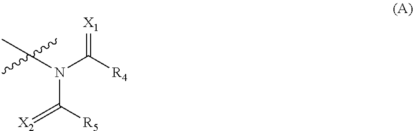

wherein

X₁ and X₂ are independently O or S;

R₄ and R₅ are independently H, OH, NH₂, C₁-C₆ alkyl or C₁-C₆ alkoxy, or

R₄ and R₅, together with the structure —C—N—C— according to formula (A), form an optionally substituted, 5-, 6-, or 7-membered saturated or partially unsaturated ring, wherein said ring is optionally fused to a 5- or 6-membered, optionally substituted ring which is a C₅-C₆ cycloalkyl, an aryl or a heterocycle comprising one or more heteroatoms independently being N, O or S.

Preferably, R₂ is a residue of formula (IIb)

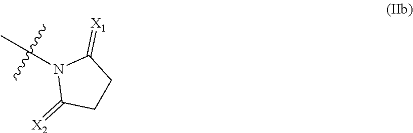

Preferably, R₂ is a residue of formula (IIc)

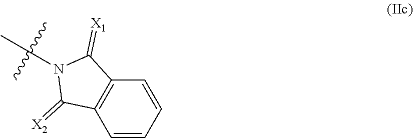

Preferably, X1 is O and X2 is O.

Preferably, n is 1 and R₂ is a residue of formula (B)

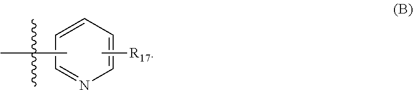

Preferably, R17 is selected from the group consisting of F, Cl, Br, I, NO2, CHO, COOH, COO—(C1-C6)alkyl, CN and COCl.

Preferably, n is 1 and R₂ is a residue of formula (C)

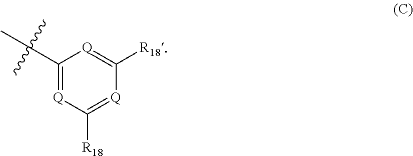

Preferably, R18 and R18′ are independently F, Cl, Br, I, or C1-C6 alkoxy and each Q is independently C or N, wherein at least one Q is N.

Preferably, n is 1 and R₂ is a residue of formula (D)

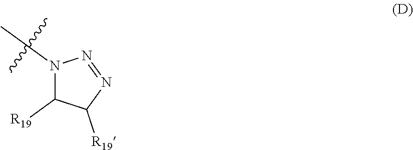

wherein R₁₉ and R_{19′} are independently H, OH, NH₂, C₁-C₆ alkyl optionally substituted with at least one of OH and NH₂, or C₁-C₆ alkoxy optionally substituted with at least one of OH and NH₂; or

R₁₉ and R_{19′} taken together form an optionally substituted 5-, 6-, or 7-membered saturated or partially unsaturated or aromatic ring, wherein the aromatic ring is preferably benzo,

wherein the ring is optionally fused to a 5- or 6-membered, optionally substituted ring which is a C₅-C₆ cycloalkyl, an aryl, preferably benzo, or a heterocycle comprising one or more heteroatoms independently being N, O or S, the 5- or 6-membered optionally substituted ring preferably being heteroaryl. Preferably, the substituent of the optionally substituted 5-, 6-, or 7-membered saturated or partially unsaturated or aromatic ring is at least a substituent, preferably one substituent, selected from the group consisting of OH, C1-C6 alkoxy, aryl, heteroaryl, C3-C6 cycloal-kyl, F, Cl, Br, I, COOH, CHO, C(O)(C1-C6 alkyl), C(O)(aryl), COO(C1-C6 alkyl), COONH2, COONH(C1-C6 alkyl), CN, NO2, —NH2, NR27R28, wherein R27 and R28 are independently selected from the group consisting of H, C1-C6 alkyl, C1-C6 alkoxy, aryl, heteroaryl, and wherein aryl at each occurrence is preferably phenyl. Preferably, the aromatic ring formed by R19 and R19′ taken together is a benzo substituted with at least one, preferably with one substituent, wherein the substituent is selected from the group consisting of OH, C1-C6 alkoxy, aryl, heteroaryl, C3-C6 cycloalkyl, F, Cl, Br, I, COOH, CHO, C(O)(C1-C6 alkyl), C(O)(aryl), COO(C1-C6 alkyl), COONH2, COONH(C1-C6 alkyl), CN, NO2, —NH2, NR27R28, wherein R27 and R28 are independent-ly selected from the group consisting of H, C1-C6 alkyl, C1-C6 alkoxy, aryl, heteroaryl, and wherein aryl at each occurrence is preferably phenyl.

Preferably, n is 1, Y is O and R2 is

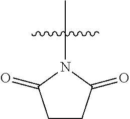 [SMILES: *N1C(=O)CCC1=O]

Preferably, n is 0 and R2 is Cl.

In a preferred aspect of the present invention, the compound of formula (III) is the compound of formula (III′)

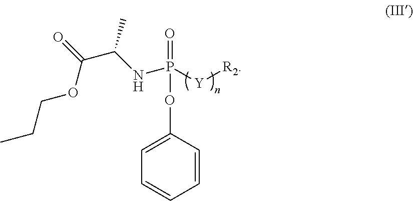

Also in a preferred aspect of the present invention, the compound of formula (III) is the compound of formula (III′)

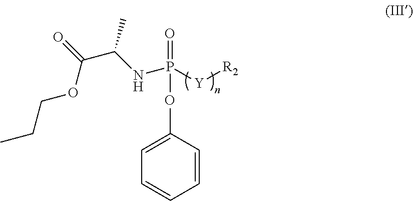

and (Y)ₙR₂ is as described herein above, i.e. a suitable leaving group for a nucleophilic substitution reaction.

Also preferably, the compound of formula (III) is the compound of formula (III″) or the compound of formula (iii″), preferably the compound of formula (III″)

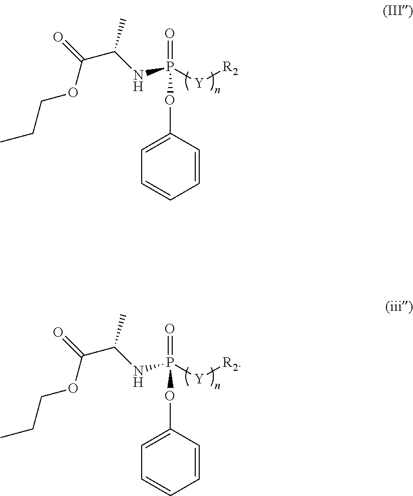

Preferably, the compound of formula (III) is the compound of formula (III″) or the compound of formula (iii″), preferably the compound of formula (III″)

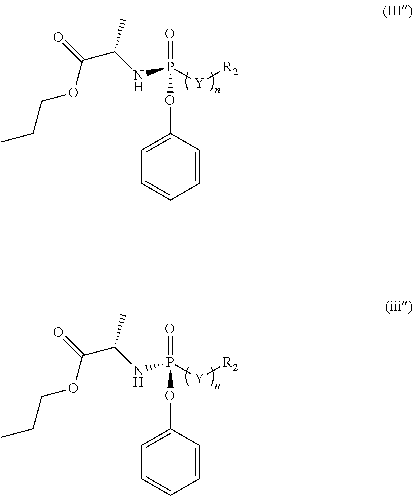

and (Y)ₙR₂ is as described herein above, i.e. a suitable leaving group for a nucleophilic substitution reaction

In a fourth embodiment, the present invention relates to compositions, in particular to pharmaceutical compositions, comprising at least one compound of formula (I). Preferably, the compound of formula (I) is the compound of formula (Ia), the compound of formula (I′a), the compound of formula (I″a) or the compound of formula (i″a), preferably the compound of formula (I″a).

Preferably, the composition further comprises a pharmaceutically acceptable excipient. Preferably, the at least one pharmaceutically acceptable excipient is selected from the group consisting of carriers, fillers, diluents, lubricants, sweeteners, stabilizing agents, solubilizing agents, antioxidants and preservatives, flavouring agents, binders, colorants, osmotic agents, buffers, surfactants, disintegrants, granulating agents, coating materials and combinations thereof. Preferably, the at least one pharmaceutically acceptable excipient is selected from the group consisting of mannitol, microcrystalline cellulose, croscarmellose sodium, colloidal anhydrous silica and magnesium stearate. Preferably, the compositions comprising at least one compound of formula (I) further comprise another antiviral agent. Regarding the another antiviral agent, no limitation exists as to its nature as long as the desired therapeutic effect is achieved. Preferably, the another antiviral agent is an NS5A inhibitor selected from the list consisting of Ledipasvir, Daclatasvir, Elbasvir, Odalasvir, Ombitasvir, Ravidasvir, Samatasvir, Ravidasvir and Velpatasvir, preferably wherein the another antiviral agent is Ledipasvir or Daclatasvir. More preferably, the another antiviral agent is Ledipasvir. More preferably, the another antiviral agent is Daclatasvir. More Preferably, the another antiviral agent is Ravidasvir.

Preferably, the compound of formula (I) is present in an effective and/or predetermined amount.

Preferably, the effective and/or predetermined amount is about 400 mg of the compound of formula (I), more preferably 400 mg of the compound of formula (I). Also preferably, the compound of formula (I) is present in an amount of from 25 to 60 weight-%, preferably of from 25 to 50 weight-%, preferably of from 30 to 45 weight-%, preferably of from 30 to 35 weight-%, more preferably about 33 weight-%, based on the total weight of the composition. In a particularly preferred aspect, the compound of formula (I) in any of the compositions described herein above is the compound of formula (I″a) as described above.

In a fifth embodiment, the present invention relates to the use of the compounds of formula (I) or to the compositions comprising at least one compound of formula (I) described herein above. Preferably, the present invention relates to the use of a compound of formula (I) or a composition comprising at least one compound of formula (I) as described herein above for the treatment of an infection in a human by a virus selected from HCV, West Nile virus, yellow fever virus, dengue virus, rhinovirus, polio virus, HAV, bovine viral diarrhea or Japanese encephalitis virus. More preferably, the virus is HCV.

Also preferably, the present invention relates to the use of a compound of formula (I) or a composition comprising at least one compound of formula (I) as described herein above for use in therapy.

In particular, the present invention relates to the use of a compound of formula (I) as described herein above for use in the treatment of an infection in a human by a virus selected from HCV, West Nile virus, yellow fever virus, dengue virus, rhinovirus, polio virus, HAV, bovine viral diarrhea or Japanese encephalitis virus. Preferably, the virus is HCV.

In a particularly preferred aspect, the present invention relates to the use of a compound of formula (I) or a composition comprising at least one compound of formula (I), wherein the compound of formula (I) is the compound of formula (I″a) or the compound of formula (i″a), preferably the compound of formula (I″a)

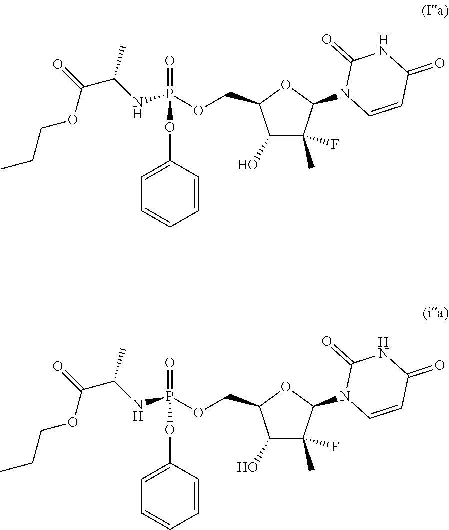 [SMILES: CCCOC(=O)[C@H](C)N[P@](=O)(OC[C@H]1O[C@@H](n2ccc(=O)[nH]c2=O)[C@](C)(F)[C@@H]1O)Oc1ccccc1 ; CCCOC(=O)[C@H](C)N[P@@](=O)(OC[C@H]1O[C@@H](n2ccc(=O)[nH]c2=O)[C@](C)(F)[C@@H]1O)Oc1ccccc1]

Also particularly preferred is the above-described use further comprising administering to the subject an effective amount of another antiviral agent when the compound of formula (I) is the compound of formula (I″a) or the compound of formula (i″a), preferably when it is the compound of formula (I″a). Regarding the another antiviral agent, no limitation exists as to its nature as long as the desired therapeutic effect is achieved. Preferably, the another antiviral agent is an NSSA inhibitor selected from the list consisting of Ledipasvir, Daclatasvir, Elbasvir, Odalasvir, Ombitasvir, Ravidasvir, Samatasvir, Ravidasvir and Velpatasvir, preferably wherein the another antiviral agent is Ledipasvir or Daclatasvir. More preferably, the another antiviral agent is Ledipasvir. More preferably, the another antiviral agent is Daclatasvir. More preferably, the another antiviral agent is Ravidasvir.

In a sixth embodiment, the present invention relates to methods of treatment comprising the use of a compound of formula (I) or of a composition comprising at least one compound of formula (I) as described herein above. Thus, the present invention relates to a method of treating a human infected by hepatitis C virus comprising administering to the subject an effective amount of a compound of formula (I), a compound of formula (Ia), a compound of formula (I′), a compound of formula (I′a), a compound of formula (I″), a compound of formula (I″a) or a compound of formula (i″a), preferably a compound of formula (I″a) or a composition comprising of a compound of formula (I), a compound of formula (Ia), a compound of formula (I′), a compound of formula (I′a), a compound of formula (I″), a compound of formula (I″a) or a compound of formula (i″a), preferably a compound of formula (I″a). Preferably, the method comprises administering the compound or the composition to the human once, twice, three times or four times daily, preferably once daily. Preferably, the method comprises administering the compound or the composition to the human in a tablet or a capsule form, preferably in a tablet form. Preferably, the human is infected with hepatitis C virus genotype 1, 2, 3, 4, 5 or 6 or a combination thereof.

The present invention is best described and illustrated by the following embodiments and combinations of embodiments as given by their respective dependencies and references.

### Compounds

1. A compound of formula (I)

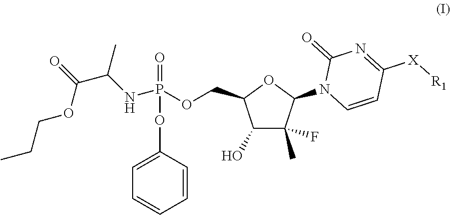

as well as isomers, stereoisomers, diastereoisomers and salts thereof, wherein X is O or NH and wherein when X is O R1 is H or a hydroxyl protecting group and when X is NH R1 is H or an amine protecting group.

2. The compound of embodiment 1, wherein X is O and R1 is hydrogen or a hydroxyl protecting group.

3. The compound of any of embodiments 1 or 2, wherein R1 is a hydroxyl protecting group selected from the group consisting of alkyl, silyl, benzyl and ester.

4. The compound of any of embodiments 1 to 3, wherein X is O and R1 is a silyl protecting group, preferably trimethylsilyl (TMS), triethylsilyl (TES), triisopropylsilyl (TIPS), dimethylisopropylsilyl (DMIPS), dimethylhexylsilyl (TDS), t-butyldimethylsilyl (TBS, TBDMS), t-butyldiphenylsilyl (TBDPS), triphenylsilyl (TPS), diphenylmethylsilyl (DPMS) or di-t-butylmethylsilyl (DTBMS).

5. The compound of any of embodiments 1 to 3, wherein X is O and R1 is an alkyl protecting group, preferably ethyl.

6. The compound of any of embodiments 1 to 3, wherein X is O and R1 is a benzyl protecting group.

7. The compound of any of embodiments 1 to 3, wherein X is O and R1 is an ester protecting group, preferably formate, acetate, benzoate, p-methoxybenzoate, benzoylformate, chloroacetate, dichloroacetate, trichloroacetate, trifluoroacetate, methoxyacetate, phenoxyacetate, p-chlorophenoxyacetate, phenylacetate, diphenylacetate, pivalate, benzoate and picolinate, more preferably acetate, benzoate, pivalate or p-methoxybenzoate.

8. The compound of embodiment 1, wherein X is NH and R1 is hydrogen or an amine protecting group.

9. The compound of any of embodiments 1 or 8, wherein X is NH and R1 is an amine protecting group selected from the group consisting of benzyl, amide and carbamate.

10. The compound of any of embodiments 8 or 9, wherein X is NH and R1 is a benzyl protecting group.

11. The compound of any of embodiments 8 or 9, wherein X is NH and R1 (NH) is an amide protecting group, preferably acetyl, chloroacetyl, benzoyl, formyl, trichloroacetyl, trifluoroacetyl, phenylacetyl, more preferably benzoyl.

12. The compound of any of embodiments 8 or 9, wherein X is NH and R1 is a carbamate protecting group, preferably methyl carbamate, ethyl carbamate, 9-fluorenylmethyl carbamate (Fmoc), t-butyl carbamate (Boc), allyl carbamate (Alloc) or vinyl carbamate (Voc).

13. The compound of any of embodiments 1 to 12, wherein the compound of formula (I) is the compound of formula (Ia) or the compound of formula (Ib)

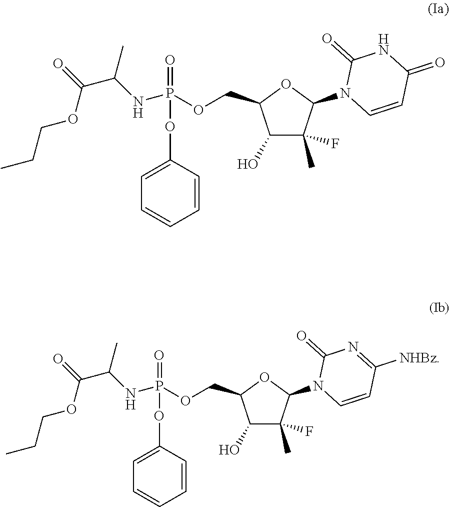

14. The compound of any of embodiments 1 to 13, wherein the compound of formula (I) is the compound of formula (Ia)

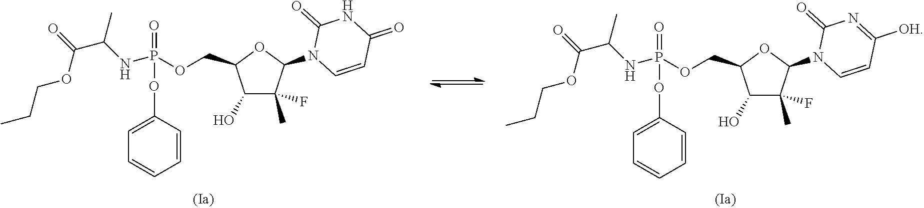 [SMILES: CCCOC(=O)C(C)NP(=O)(OC[C@H]1O[C@@H](n2ccc(=O)[nH]c2=O)[C@](C)(F)[C@@H]1O)Oc1ccccc1 ; CCCOC(=O)C(C)NP(=O)(OC[C@H]1O[C@@H](n2ccc(O)nc2=O)[C@](C)(F)[C@@H]1O)Oc1ccccc1]

15. The compound of any of embodiments 1 to 12, wherein the compound of formula (I) is the compound of formula (I′)

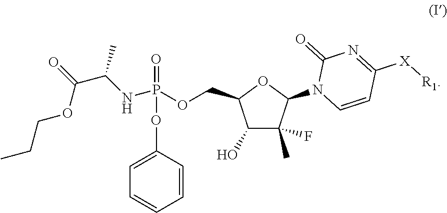

16. The compound of embodiment 15, wherein the compound of formula (I′) is the compound of formula (I′a) or the compound of formula (I′b)

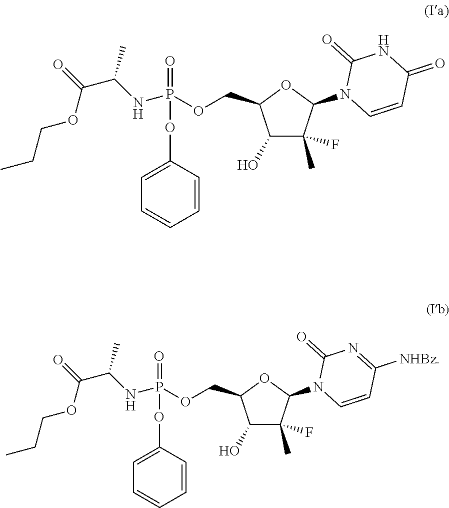

17. The compound of any of embodiments 15 or 16, wherein the compound of formula (I′) is the compound of formula (I′a)

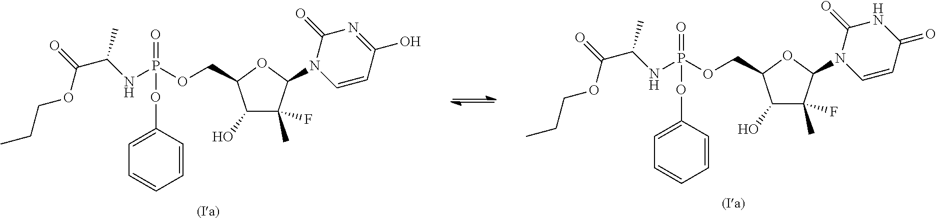 [SMILES: CCCOC(=O)[C@H](C)NP(=O)(OC[C@H]1O[C@@H](n2ccc(O)nc2=O)[C@](C)(F)[C@@H]1O)Oc1ccccc1 ; CCCOC(=O)[C@H](C)NP(=O)(OC[C@H]1O[C@@H](n2ccc(=O)[nH]c2=O)[C@](C)(F)[C@@H]1O)Oc1ccccc1]

18. The compound of any of embodiments 1 to 12, wherein the compound of formula (I) is the compound of formula (I″) or the compound of formula (i″), preferably the compound of formula (I″)

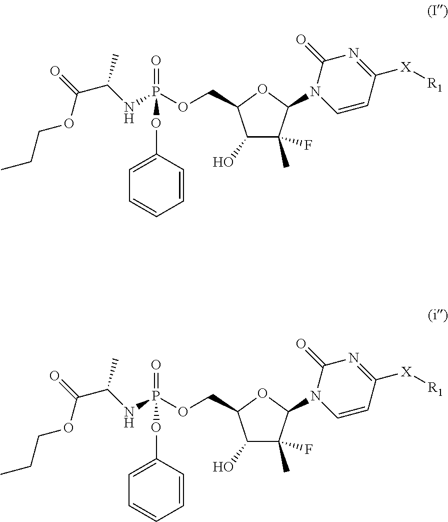

19. The compound of embodiment 18, wherein the compound of formula (I″) is the compound of formula (I″a), the compound of formula (I″b), the compound of formula (i″a) or the compound of formula (i″b), preferably the compound of formula (I″a) or the compound of formula (I″b)

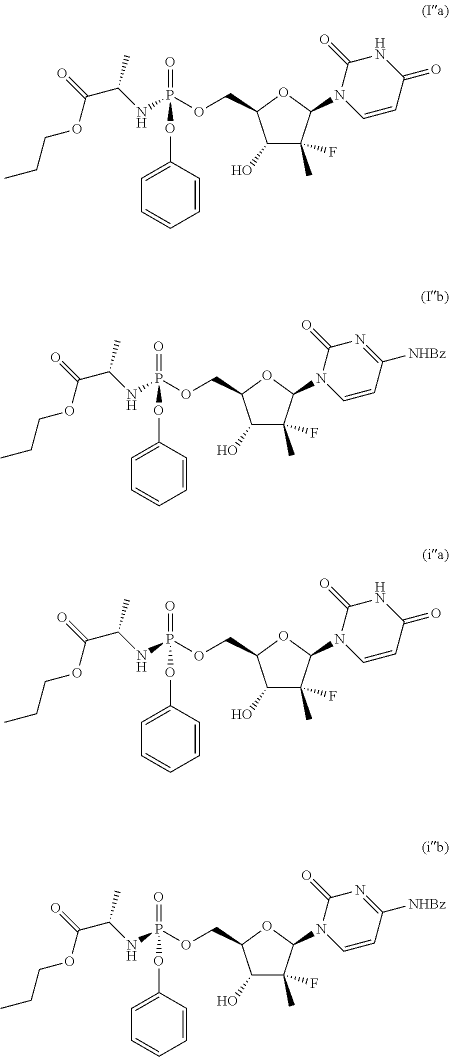

20. The compound of any of embodiments 18 or 19, wherein the compound of formula (I″) is the compound of formula (I″a) or the compound of formula (i″a), preferably the compound of formula (I″a)

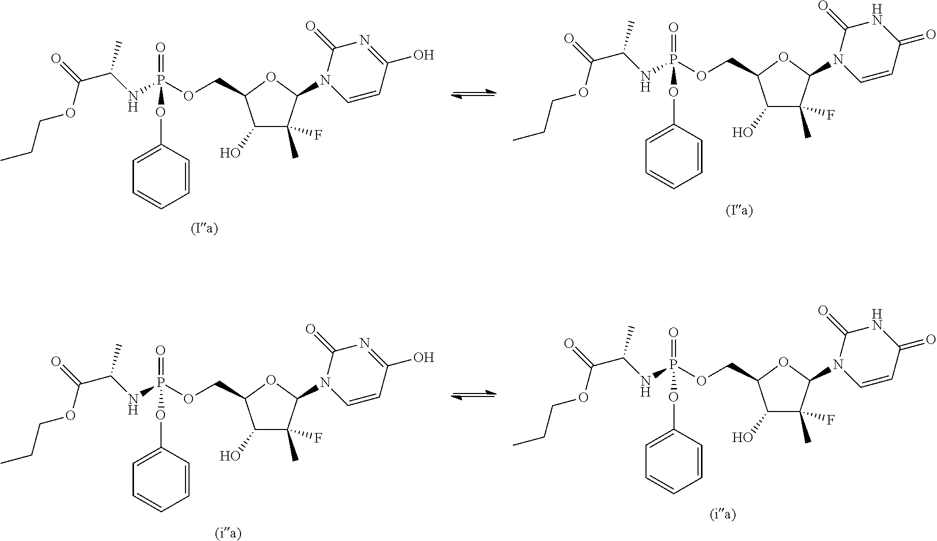 [SMILES: CCCOC(=O)[C@H](C)N[P@](=O)(OC[C@H]1O[C@@H](n2ccc(O)nc2=O)[C@](C)(F)[C@@H]1O)Oc1ccccc1 ; CCCOC(=O)[C@H](C)N[P@](=O)(OC[C@H]1O[C@@H](n2ccc(=O)[nH]c2=O)[C@](C)(F)[C@@H]1O)Oc1ccccc1 ; CCCOC(=O)[C@H](C)N[P@@](=O)(OC[C@H]1O[C@@H](n2ccc(O)nc2=O)[C@](C)(F)[C@@H]1O)Oc1ccccc1 ; CCCOC(=O)[C@H](C)N[P@@](=O)(OC[C@H]1O[C@@H](n2ccc(=O)[nH]c2=O)[C@](C)(F)[C@@H]1O)Oc1ccccc1]

21. The compound of any of embodiments 18 to 20, wherein the compound of formula (I″) is the compound of formula (I″a)

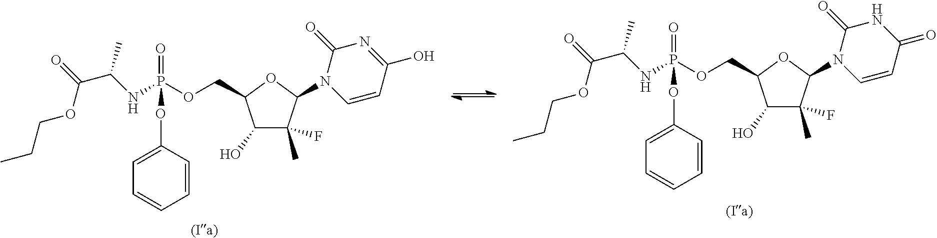 [SMILES: CCCOC(=O)[C@H](C)N[P@](=O)(OC[C@H]1O[C@@H](n2ccc(O)nc2=O)[C@](C)(F)[C@@H]1O)Oc1ccccc1 ; CCCOC(=O)[C@H](C)N[P@](=O)(OC[C@H]1O[C@@H](n2ccc(=O)[nH]c2=O)[C@](C)(F)[C@@H]1O)Oc1ccccc1]

22. The compound of any of embodiments 1 to 21 in amorphous, crystalline or pseudo-crystalline form or mixtures thereof.

23. The compound of any of embodiments 1 to 22 in crystalline form.

24. The compound of formula (I″a) in crystalline form, preferably the compound of formula (I″a) according to any of embodiments 20 or 21 in crystalline form.

25. The compound of embodiment 24 having an X-ray powder diffraction pattern comprising reflections at 2-theta angles of (5.1±0.2)°, (6.9±0.2)°, (9.2±0.2)°, (16.3±0.2)°, (20.4±0.2)° when measured at a temperature in the range of from 15 to 25° C. with Cu-K_{alpha1,2} radiation having a wavelength of 0.15419 nm.

26. The compound of any of embodiments 24 or 25 comprising further reflections at 2-theta angles of (8.0±0.2)°, (15.3±0.2)°, (16.7±0.2)°, (17.9±0.2)°, (25.6±0.2)° when measured at a temperature in the range of from 15 to 25° C. with Cu-K_{alpha1,2} radiation having a wavelength of 0.15419 nm.

27. The compound of any of embodiments 24 to 26 having a monoclinic space group symmetry and the following unit cell parameters as determined by an X-ray single crystal structure analysis at 173K:

a=12.8656 Angstrom

b=6.0028 Angstrom

c=17.5417 Angstrom

α=90°

β=98.397°

γ=90°

28. The compound of any of embodiments 24 to 27 having a melting point in the range of from 77.5° C. to 82.7° C. when measured via differential scanning calorimetry at a heating rate of 10K/min.

### Processes

29. A process for the preparation of a compound of formula (I) according to any of embodiments 1 to 28 comprising
(vi) providing a compound of formula (II) or a mixture comprising the compound of formula (II)
(vii) reacting the compound of formula (II) with a compound of formula (III) to get a compound of formula (I)
(viii) optionally isolating the compound of formula (I)

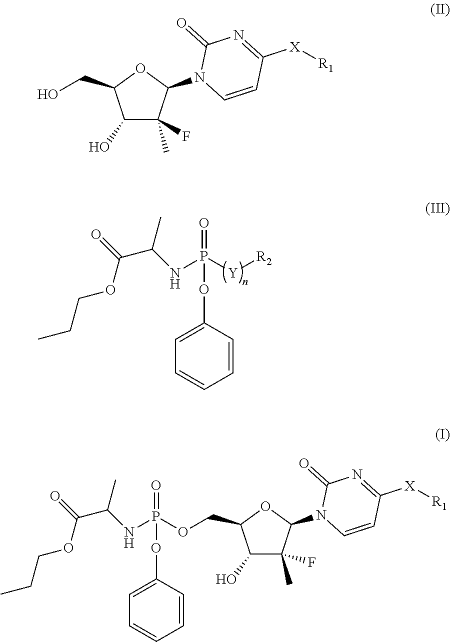

wherein (Y)ₙR₂ is a suitable leaving group for a nucleophilic substitution reaction.

30. The process of embodiment 29, wherein n is 0 or 1 and wherein Y is O, N or S.

31. The process of any of embodiments 29 or 30, wherein n is 1 and R₂ is alkyl, aryl, or heteroaryl, each optionally substituted with one or more electron-withdrawing groups, preferably aryl optionally substituted with one or more electron-withdrawing groups, more preferably phenyl optionally substituted with one or more electron-withdrawing groups.

32. The process of embodiment 31, wherein n is 1 and R₂ is phenyl substituted with one or more electron-withdrawing groups, wherein the one or more electron-withdrawing groups are preferably F, Cl, Br, I, or NO₂.

33. The process of any of embodiments 29, 30, 31 or 32, wherein n is 1, Y is O or S and R₂ is

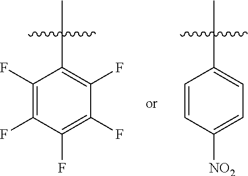 [SMILES: *c1c(F)c(F)c(F)c(F)c1F ; *c1ccc([N+](=O)[O-])cc1]

34. The process of any of embodiments 31 to 33, wherein R₂ is

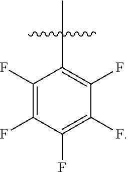 [SMILES: *c1c(F)c(F)c(F)c(F)c1F]

35. The process of any of embodiments 29 or 30, wherein n is 1 and R₂ is a residue of formula (A)

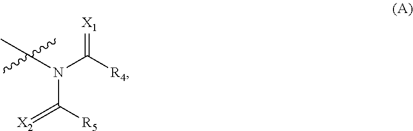

a residue of formula (B)

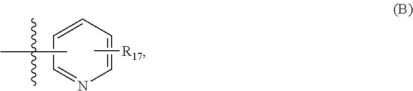

a residue of formula (C)

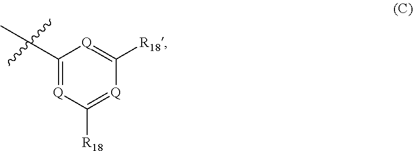

or a residue of formula (D)

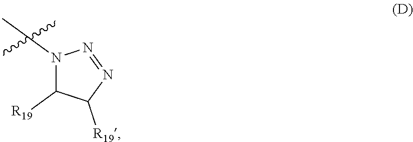

wherein at each occurrence

X₁ and X₂ are independently O or S;

R₄ and R₅ are independently H, OH, NH₂, C₁-C₆ alkyl or C₁-C₆ alkoxy, or

R₄ and R₅, together with the structure —C—N—C— according to formula (A), form an optionally substituted, 5-, 6-, or 7-membered saturated or partially unsaturated ring, wherein said ring is optionally fused to a 5- or 6-membered, optionally substituted ring which is a C₅-C₆ cycloalkyl, an aryl or a heterocycle comprising one or more heteroatoms independently being N, O or S;

R₁₇ is an electron-withdrawing group, preferably F, Cl, Br, I, NO₂, CHO, COOH, COO—(C₁-C₆)alkyl, CN, or COCl;

R₁₈ and R_{18′} are independently F, Cl, Br, I, or C₁-C₆ alkoxy; each Q is independently C or N, wherein at least one Q is N;

R₁₉ and R_{19′} are independently H, OH, NH₂, C₁-C₆ alkyl optionally substituted with at least one of OH and NH₂, or C₁-C₆ alkoxy optionally substituted with at least one of OH and NH₂; or

R₁₉ and R_{19′} taken together form an optionally substituted 5-, 6-, or 7-membered saturated or partially unsaturated or aromatic ring, wherein the ring is optionally fused to a 5- or 6-membered, optionally substituted ring which is a C₅-C₆ cycloalkyl, an aryl, preferably benzo, or a heterocycle comprising one or more heteroatoms independently being N, O or S, the 5- or 6-membered optionally substituted ring preferably being heteroaryl.

36. The process of any of embodiments 29 or 30, wherein n is 0 and R₂ is a residue of formula (A1)

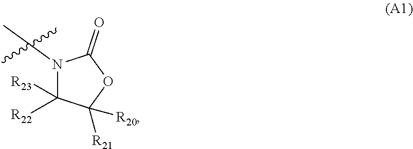

wherein R₂₀, R₂₁, R₂₂ and R₂₃ are each independently H, aryl, or C₁-C₆ alkyl optionally substituted with at least one of C₁-C₆ alkoxy optionally substituted with at least one of OH and NH₂; or

R₂₀ and R₂₂, or R₂₀ and R₂₃, or R₂₁ and R₂₂, or R₂₁ and R₂₃ when taken together form an optionally substituted 5-, 6-, or 7-membered saturated or partially unsaturated or aromatic ring which is an aryl, preferably benzo, or a heterocycle comprising one or more heteroatoms independently being N, O or S, the 5-, 6-, or 7-membered saturated or partially unsaturated or aromatic ring preferably being heteroaryl.

37. The process of any of embodiments 29, 30 or 36, wherein the substituent of the optionally substituted 5-, 6-, or 7-membered saturated or partially unsaturated or aromatic ring which is an aryl, preferably benzo, or a heterocycle comprising one or more heteroatoms independently being N, O or S, is at least a substituent, preferably one substituent, selected from the group consisting of OH, C1-C6 alkoxy, aryl, heteroaryl, C3-C6 cycloalkyl, F, Cl, Br, I, COOH, CHO, C(O)(C1-C6 alkyl), C(O)(aryl), COO(C1-C6 alkyl), COONH2, COONH(C1-C6 alkyl), CN, NO2, —NH2, NR27R28, wherein

R27 and R28 are independently selected from the group consisting of H, C1-C6 alkyl, C1-C6 alkoxy, aryl, heteroaryl, and wherein aryl at each occurrence is preferably phenyl.

38. The process of any of embodiments 28, 29, 35 or 36, wherein the aromatic ring is a benzo substituted with at least one, preferably with one substituent, wherein the substituent is selected from the group consisting of OH, C1-C6 alkoxy, aryl, heteroaryl, C3-C6 cycloalkyl, F, Cl, Br, I, COOH, CHO, C(O)(C1-C6 alkyl), C(O)(aryl), COO(C1-C6 alkyl), COONH2, COONH(C1-C6 alkyl), CN, NO2, —NH2, NR27R28, wherein R27 and R28 are independently selected from the group consisting of H, C1-C6 alkyl, C1-C6 alkoxy, aryl, heteroaryl, and wherein aryl at each occurrence is preferably phenyl.

39. The process of any of embodiments 29, 30 or 36, wherein R₂₂ and R₂₃ are each independently H, aryl, or C1-C6 alkyl substituted with at least one of C1-C6 alkoxy optionally substituted with at least one of OH and NH2.

40. The process of any of embodiments 29, 30 or 35, wherein n is 1 and R₂ is a residue of formula (A)

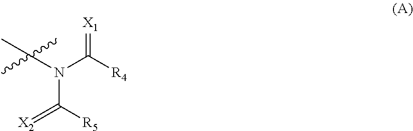

wherein

X₁ and X₂ are independently O or S;

R₄ and R₅ are independently H, OH, NH₂, C₁-C₆ alkyl or C₁-C₆ alkoxy, or

R₄ and R₅, together with the structure —C—N—C— according to formula (A), form an optionally substituted, 5-, 6-, or 7-membered saturated or partially unsaturated ring, wherein said ring is optionally fused to a 5- or 6-membered, optionally substituted ring which is a C₅-C₆ cycloalkyl, an aryl or a heterocycle comprising one or more heteroatoms independently being N, O or S.

41. The process of any of embodiments 29, 30, 35 or 38, wherein R₂ is a residue of formula (IIb)

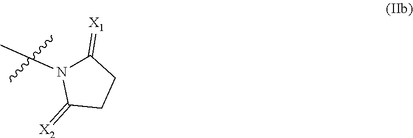

42. The process of any of embodiments 29, 30, 35 or 38, wherein R₂ is a residue of formula (IIc)

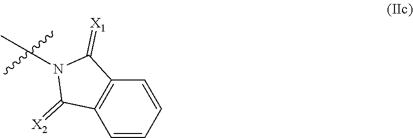

43. The process of any of embodiments 29, 30, 35 or 40 to 42, wherein X1 is O and X2 is O.

44. The process of any of embodiments 29, 30 or 35, wherein n is 1 and R₂ is a residue of formula (B)

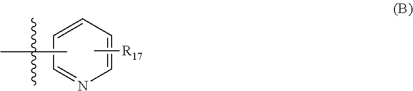

45. The process of any of embodiments 29, 30, 35 or 44, wherein R17 is selected from the group consisting of F, Cl, Br, I, NO2, CHO, COOH, COO—(C1-C6)alkyl, CN and COCl.

46. The process of any of embodiments 29, 30 or 35, wherein n is 1 and R₂ is a residue of formula (C)

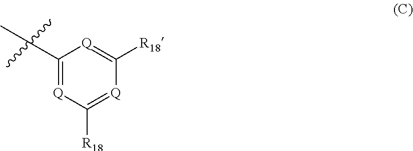

47. The process of any of embodiments 29, 30, 35 or 46, wherein R18 and R18′ are independently F, Cl, Br, I, or C1-C6 alkoxy and each Q is independently C or N, wherein at least one Q is N.

48. The process of any of embodiments 29, 30 or 35, wherein n is 1 and R₂ is a residue of formula (D)

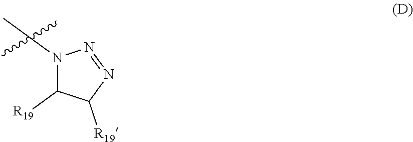

wherein R₁₉ and R_{19′} are independently H, OH, NH₂, C₁-C₆ alkyl optionally substituted with at least one of OH and NH₂, or C₁-C₆ alkoxy optionally substituted with at least one of OH and NH₂; or

R₁₉ and R_{19′} taken together form an optionally substituted 5-, 6-, or 7-membered saturated or partially unsaturated or aromatic ring, wherein the aromatic ring is preferably benzo,

wherein the ring is optionally fused to a 5- or 6-membered, optionally substituted ring which is a C₅-C₆ cycloalkyl, an aryl, preferably benzo, or a heterocycle comprising one or more heteroatoms independently being N, O or S, the 5- or 6-membered optionally substituted ring preferably being heteroaryl.

49. The process of any of embodiments 29, 30, 35 or 48, wherein the substituent of the optionally substituted 5-, 6-, or 7-membered saturated or partially unsaturated or aromatic ring is at least a substituent, preferably one substituent, selected from the group consisting of OH, C1-C6 alkoxy, aryl, heteroaryl, C3-C6 cycloal-kyl, F, Cl, Br, I, COOH, CHO, C(O)(C1-C6 alkyl), C(O)(aryl), COO(C1-C6 alkyl), COONH2, COONH(C1-C6 alkyl), CN, NO2, —NH2, NR27R28, wherein R27 and R28 are independently selected from the group consisting of H, C1-C6 alkyl, C1-C6 alkoxy, aryl, heteroaryl, and wherein aryl at each occurrence is preferably phenyl.

50. The process of any of embodiments 29, 30, 35, 48 or 49, wherein the aromatic ring formed by R19 and R19′ taken together is a benzo substituted with at least one, preferably with one substituent, wherein the substituent is selected from the group consisting of OH, C1-C6 alkoxy, aryl, heteroaryl, C3-C6 cycloalkyl, F, Cl, Br, I, COOH, CHO, C(O)(C1-C6 alkyl), C(O)(aryl), COO(C1-C6 alkyl), COONH2, COONH(C1-C6 alkyl), CN, NO2, —NH2, NR27R28, wherein R27 and R28 are independent-ly selected from the group consisting of H, C1-C6 alkyl, C1-C6 alkoxy, aryl, heteroaryl, and wherein aryl at each occurrence is preferably phenyl.

51. The process of embodiment 29 or 30, wherein n is 1, Y is O and R2 is

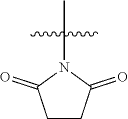 [SMILES: *N1C(=O)CCC1=O]

52. The process of embodiment 29 or 30, wherein n is 0 and R2 is Cl.

53. The process of any of embodiments 29 to 52, wherein X is O and R1 is hydrogen.

54. The process of any of embodiments 29 to 52, wherein X is NH and R1 is hydrogen.

55. The process of any of embodiments 29 to 52, wherein X is O and R1 is a hydroxyl protecting group.

56. The process of embodiment 55, wherein X is O and R1 is a hydroxyl protecting group selected from the group consisting of alkyl, silyl, benzyl and ester.

57. The process of embodiment 55 or 56, wherein X is O and R1 is a silyl protecting group, preferably trimethylsilyl (TMS), triethylsilyl (TES), triisopropylsilyl (TIPS), dimethylisopropylsilyl (DMIPS), dimethylhexylsilyl (TDS), t-butyldimethylsilyl (TBS, TBDMS), t-butyldiphenylsilyl (TBDPS), triphenylsilyl (TPS), diphenylmethylsilyl (DPMS) or di-t-butylmethylsilyl (DTBMS).

58. The process of embodiment 55 or 56, wherein X is O and R1 is an alkyl protecting group, preferably ethyl.

59. The process of embodiment 55 or 56, wherein X is O and R1 is a benzyl protecting group.

60. The process of embodiment 55 or 56, wherein X is O and R1 is an ester protecting group, preferably formate, acetate, benzoate, p-methoxybenzoate, benzoylformate, chloroacetate, dichloroacetate, trichloroacetate, trifluoroacetate, methoxyacetate, phenoxyacetate, p-chlorophenoxyacetate, phenylacetate, diphenylacetate, pivalate, benzoate and picolinate, more preferably acetate, benzoate, pivalate or p-methoxybenzoate

61. The process of any of embodiments 29 to 52, wherein X is NH and R1 is an amine protecting group.

62. The process of embodiment 61, wherein X is NH and R1 is an amine protecting group selected from the group consisting of benzyl, amide and carbamate.

63. The process of embodiment 61 or 62, wherein X is NH and R1 is a benzyl protecting group.

64. The process of embodiment 61 or 62, wherein X is NH and R1 is an amide protecting group, preferably acetyl, chloroacetyl, benzoyl, formyl, trichloroacetyl, trifluoroacetyl, phenylacetyl, more preferably benzoyl.

65. The process of embodiment 61 or 62, wherein X is NH and R1 is a carbamate protecting group, preferably methyl carbamate, ethyl carbamate, 9-fluorenylmethyl carbamate (Fmoc), t-butyl carbamate (Boc), allyl carbamate (Alloc), vinyl carbamate (Voc).

66. The process of any of embodiments 29 to 65, wherein the compound of formula (I) is the compound of formula (Ia) or (Ib)

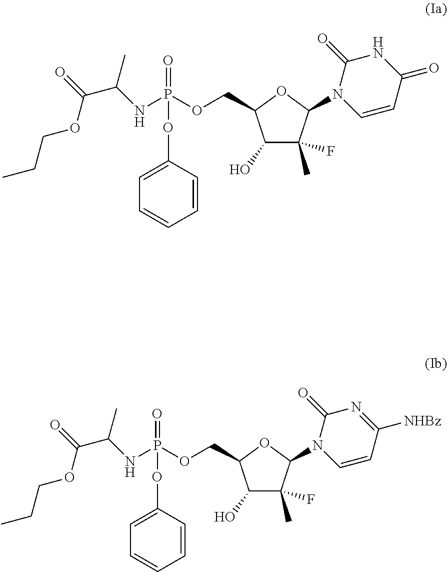

67. The process of embodiment 66, wherein the compound of formula (I) is the compound of formula (Ia)

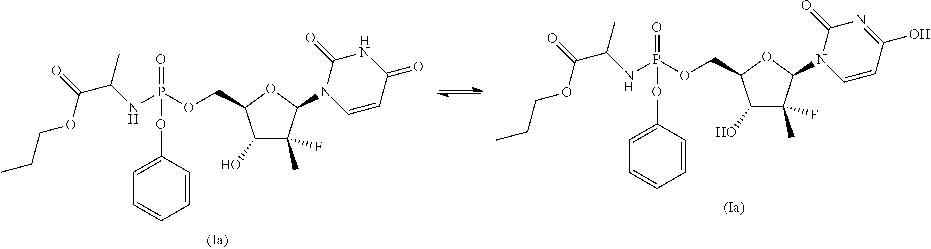 [SMILES: CCCOC(=O)C(C)NP(=O)(OC[C@H]1O[C@@H](n2ccc(=O)[nH]c2=O)[C@](C)(F)[C@@H]1O)Oc1ccccc1 ; CCCOC(=O)C(C)NP(=O)(OC[C@H]1O[C@@H](n2ccc(O)nc2=O)[C@](C)(F)[C@@H]1O)Oc1ccccc1]

68. The process of any of embodiments 29 to 67, wherein the compound of formula (I) is the compound of formula (I′) and wherein the compound of formula (III) is the compound of formula (III′)

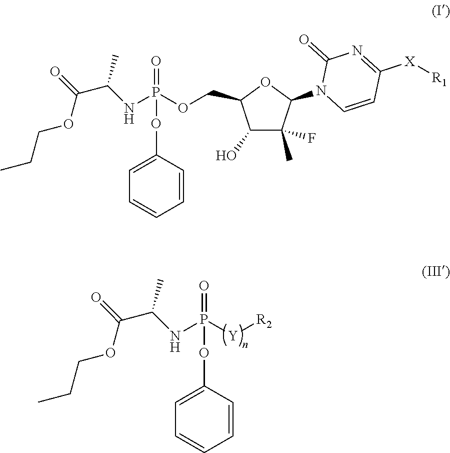

69. The process of embodiment 68, wherein the compound of formula (I′) is the compound of formula (I′a) or (I′b)

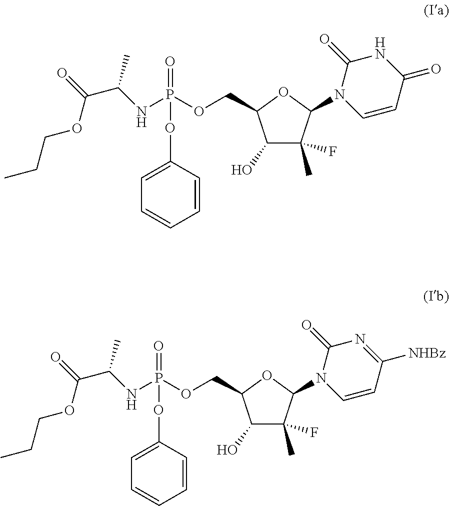

70. The process of any of embodiments 29 to 69, wherein the compound of formula (I′) is the compound of formula (I′a)

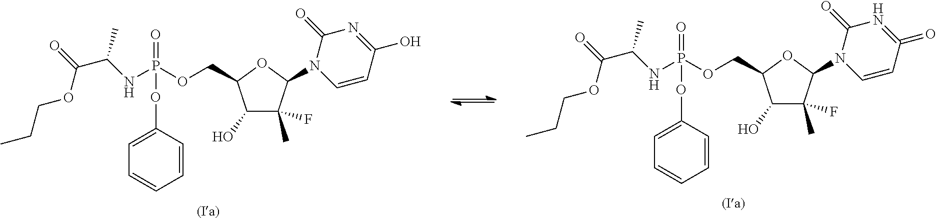

71. The process of any of embodiments 29 to 70, wherein the compound of formula (I) is the compound of formula (I″) or the compound of formula (i″) and wherein the compound of formula (III) is the compound of formula (III″) or the compound of formula (iii″)

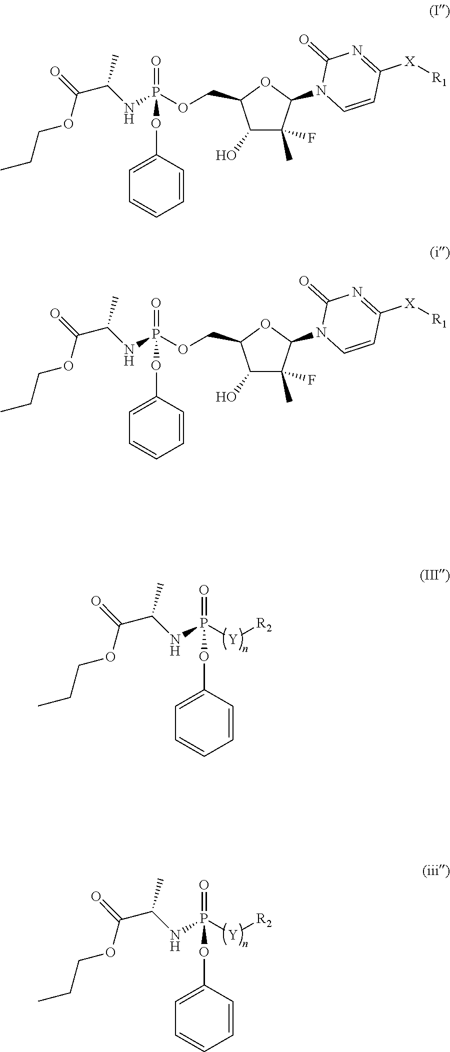

72. The process of embodiment 71, wherein the compound of formula (I″) is the compound of formula (I″a), the compound of formula (I″b), the compound of formula (i″a) or the compound of formula (i″b), preferably the compound of formula (I″a) or the compound of formula (I″b)

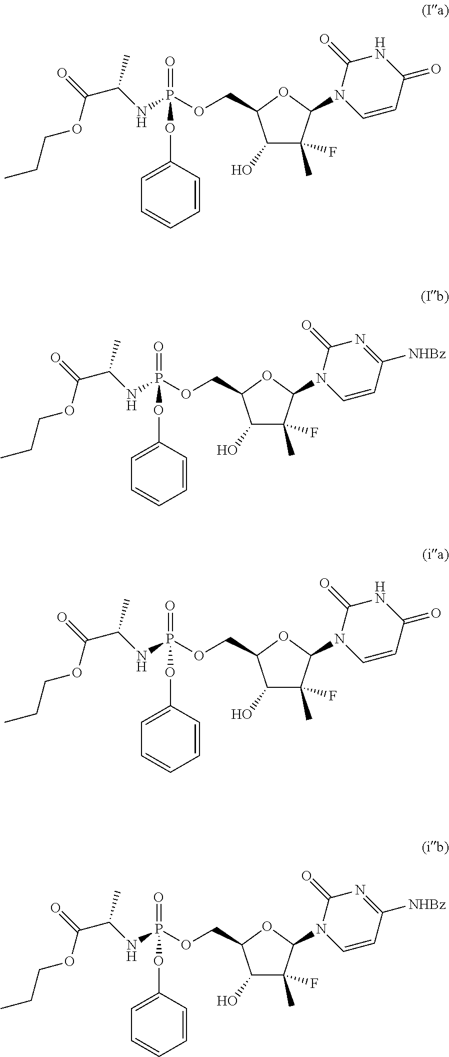

73. The process of any of embodiments 71 or 72, wherein the compound of formula (I″) is the compound of formula (I″a) or the compound of formula (i″a), preferably the compound of formula (I″a)

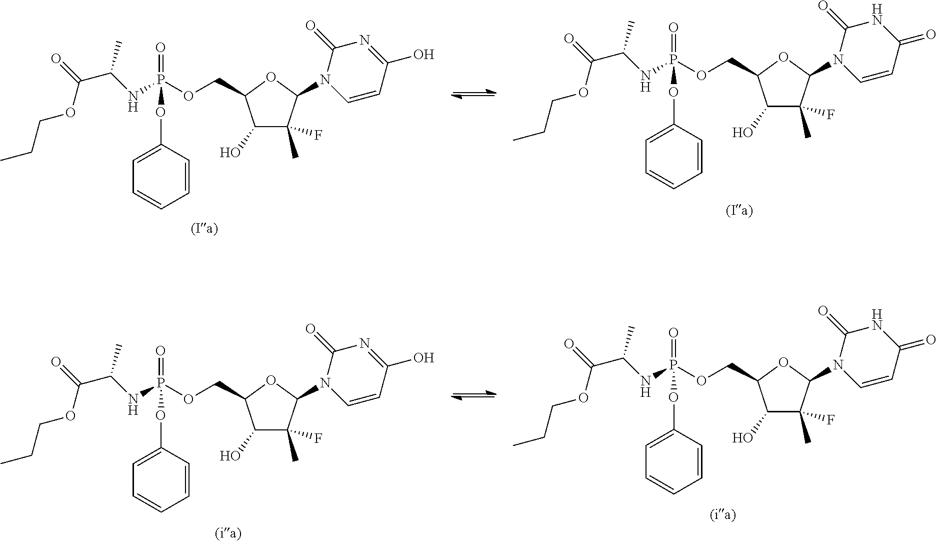 [SMILES: CCCOC(=O)[C@H](C)N[P@](=O)(OC[C@H]1O[C@@H](n2ccc(O)nc2=O)[C@](C)(F)[C@@H]1O)Oc1ccccc1 ; CCCOC(=O)[C@H](C)N[P@](=O)(OC[C@H]1O[C@@H](n2ccc(=O)[nH]c2=O)[C@](C)(F)[C@@H]1O)Oc1ccccc1 ; CCCOC(=O)[C@H](C)N[P@@](=O)(OC[C@H]1O[C@@H](n2ccc(O)nc2=O)[C@](C)(F)[C@@H]1O)Oc1ccccc1 ; CCCOC(=O)[C@H](C)N[P@@](=O)(OC[C@H]1O[C@@H](n2ccc(=O)[nH]c2=O)[C@](C)(F)[C@@H]1O)Oc1ccccc1]

74. The process of any of embodiments 29 to 73, wherein step (ii) is carried out in the presence of one or more bases.

75. The process of embodiment 74, wherein the one or more bases are organic bases.

76. The process of any of embodiments 74 or 75, wherein the one or more bases comprise an alkylmagnesium halide.

77. The process of embodiment 76, wherein the alkylmagnesium halide is tert-butylmagnesium chloride.

78. The process of embodiment 74 or 75, wherein the one or more bases are selected from the group consisting of an amine, an amidine, a heteroaromatic compound comprising a basic ring-nitrogen atom, and a mixture of two or more thereof, more preferably selected from the group consisting of ethyldiisopropylamine, triethylamine, diethylamine, 1,8-diazabicycloundec-7-ene, pyridine, quinoline, isoquinoline, acridine, pyrazine, imidazole, benzimidazole, pyrazole, and a mixture of two or more thereof.

79. The process of any of embodiments 29 to 78, wherein prior to the reaction according to (ii), the molar ratio of the one or more bases relative to the compound of formula (III) is in the range of from 0.1:1 to 5:1 wherein, if more than one base is comprised in the mixture provided in a), the molar ratio relates to the total molar amount of all bases.

80. The process of embodiment 79, wherein the molar ratio of the one or more bases relative to the compound of formula (III) is in the range of from 0.1:1 to 2:1 preferably in the range of from 0.5:1 to 1.2:1 wherein, if more than one base is comprised in the mixture provided in a), the molar ratio relates to the total molar amount of all bases.

81. The process of any of embodiments 29 to 80, wherein the mixture provided in (i) further comprises one or more solvents and one or more bases, wherein prior to the reaction according to (ii), the molar ratio of the one or more bases relative to the compound of formula (III) is in the range of from 0.1:1 to 5:1.

82. The process of any of embodiments 29 to 81, wherein step (ii) is carried out in the presence of one or more Lewis acids.

83. The process of embodiment 82, wherein the one or more Lewis acids comprise a twice positively charged ion or a three times positively charged ion.

84. The process of embodiment 82, wherein the one or more Lewis acids comprise a twice positively charged metal ion or a three times positively charged metal ion.

85. The process of any of embodiments 82 to 84, wherein the twice positively charged ion is a Zn ion, a Mg ion, a Cu ion, or an Fe ion.

86. The process of any of embodiments 82 to 85, wherein the twice positively charged ion is a Zn ion.

87. The process of any of embodiments 82 to 86, wherein the one or more Lewis acids is one or more of ZnBr2, ZnCl2, ZnI2.

88. The process of any of embodiments 82 to 87, wherein the one or more Lewis acids comprises, preferably is ZnBr2.

89. The process of any of embodiments 82 to 85, wherein the one or more Lewis acids is one or more of ZnBr2, ZnCl2, ZnI2, MgBr2, MgBr2•OEt2, CuCl2, Cu(acetylacetonate)2, and Fe(II) fumarate.

90. The process of any of embodiments 82 to 84, wherein the three times positively charged ion is a Mn ion.

91. The process of embodiment 90, wherein the one or more Lewis acids is Mn(acetylacetonate)3.

92. The process of any of embodiments 29 to 91, wherein step (ii) is carried out in a suitable solvent or suitable solvent mixture.

93. The process of embodiment 92, wherein the suitable solvent or solvent mixture consists of or comprises a solvent selected from the list consisting of methylene chloride, methyl tert-butyl ether, tetrahydrofurane, dimethylsulphoxide, dimethylformamide, and a mixture of two or more thereof.

94. The process of any of embodiments 29 to 93, wherein prior to the reaction according to (ii), the molar ratio of the compound of formula (II) relative to the compound of formula (III) is in the range of from 0.5:1 to 5:1.

95. The process of embodiment 94, wherein the molar ratio of the compound of formula (II) relative to the compound of formula (III) is in the range of from 0.8:1 to 2:1, preferably in the range of from 0.9:1 to 1.2:1.

96. The process of any of embodiments 29 to 95, wherein prior to the reaction according to (ii), the molar ratio of the Lewis acid relative to the compound of formula (III) is in the range of from 0.1:1 to 5:1.

97. The process of embodiment 96, wherein the molar ratio of the Lewis acid relative to the compound of formula (III) is in the range of from 0.2:1 to 2:1, preferably in the range of from 0.5:1 to 1.2:1.

98. The process of any of embodiments 29 to 97, wherein step (ii) is carried out at a temperature in the range of from 0 to 80° C.

99. The process of embodiment 98, wherein the temperature is in the range of from 10 to 65° C.

100. The process of any of embodiments 98 or 99, wherein the temperature is in the range of from 20 to 50° C.

101. The process of any of embodiments 29 to 100, wherein the reaction in step (ii) s carried out for a period of time in the range of from 0.5 to 48 h.

102. The process of embodiment 101, wherein the period of time is in the range of from 1 to 36 h.

103. The process of any of embodiments 101 or 102, wherein the period of time is in the range of from 2 to 24 h.

104. The process of any of embodiments 29 to 103, wherein the reaction conditions in step (ii) comprise a temperature of the mixture in the range of from 20 to 50° C., wherein according to (ii), the mixture is subjected to the reaction conditions for a period of time in the range of from 2 to 24 h.

105. The process of any of embodiments 29 to 104, wherein prior to the reaction according to (ii), the molar ratio of the compound of formula (II) relative to the compound of formula (III) is in the range of from 0.9:1 to 1.2:1, the molar ratio of the Lewis acid relative to the compound of formula (III) is in the range of from 0.5:1 to 1.2:1, and the molar ratio of the one or more bases relative to the compound of formula (III) is in the range of from 0.5:1 to 1.2:1 wherein, if more than one base is comprised in the mixture provided in a), the molar ratio relates to the total molar amount of all bases.

106. The process of any of embodiments 29 to 105, wherein X is O and R1 is a hydroxyl protecting group or wherein X is NH and R1 is an amine protecting group further comprising, after step (ii) or after optional step (iii),

(ix) removing the hydroxyl or amine protecting group to get a compound of formula (Ia), a compound of formula (I′a), a compound of formula (I″a) or a compound of formula (i″a)

(x) optionally isolating the compound of formula (Ia), the compound of formula (I′a), the compound of formula (I″a) or the compound of formula (i″a).

107. The process of embodiment 106 wherein X is O and R1 is benzyl and wherein removing the protecting group in (iv) comprises subjecting the protected compound to hydrogenolysis.

108. The process of embodiment 106 wherein X is O and R1 is an ester protecting group, preferably benzoyl and wherein removing the protecting group in (iv) comprises subjecting the protected compound to acidic, basic or reducing conditions, preferably basic or reducing conditions, preferably reducing conditions in the presence of LiAlH4.

109. The process of embodiment 106 wherein X is O and R1 is a silyl protecting group and wherein removing the protecting group in (iv) comprises subjecting the protected compound to acidic conditions.

110. The process of embodiment 106 wherein X is O and R1 is an alkyl protecting group, preferably ethyl and wherein removing the protecting group in (iv) comprises subjecting the protected compound to methanolic ammonia.

111. The process of embodiment 106 wherein X is NH and R1 is an amide protecting group, preferably benzoyl and wherein removing the protecting group in (iv) comprises subjecting the protected compound to acidic, basic or reducing conditions, preferably basic or reducing conditions, preferably reducing conditions in the presence of LiAlH4.

112. The process of embodiment 106 wherein X is NH and R1 is benzyl and wherein removing the protecting group in (iv) comprises subjecting the protected compound to hydrogenolysis.

113. The process of any of embodiments 29 to 112, wherein the compound of formula (Ia), preferably the compound of formula (I′a), more preferably the compound of formula (I″a), is obtained after step (iv) or after optional step (v).

114. The process of any of embodiments 106 to 113, wherein isolating in step (iii) or step (v) is achieved by, consists of or comprises precipitation, crystallization or chromatography.

115. The process of embodiment 114, wherein crystallization comprises seeding.

116. The process of any of embodiments 114 or 115, wherein crystallization comprises using a solvent mixture comprising dichloromethane and heptane.

117. The process of embodiment 116, wherein the dichloromethane and heptane are used in a volume ratio of from 30:30 to 60:10, preferably of from 70:20 to 30:20, preferably of from 45:25 to 55:15.

118. The process of any of embodiments 116 or 117, wherein crystallization is carried out at a temperature of from 0 to 40° C., preferably of from 20 to 30° C.

119. A process for the preparation of a compound of formula (I″a) in crystalline form comprising

(iv) providing a solution of the compound of formula (I″a) in a suitable solvent or solvent mixture,

(v) subjecting the solution of (i) to crystallization conditions

(vi) isolating the crystalline compound of formula (I″a)

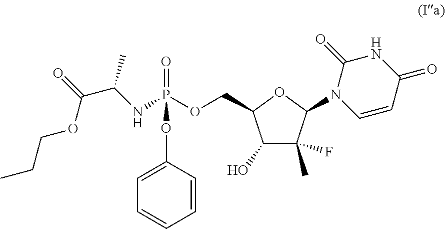 [SMILES: CCCOC(=O)[C@H](C)N[P@](=O)(OC[C@H]1O[C@@H](n2ccc(=O)[nH]c2=O)[C@](C)(F)[C@@H]1O)Oc1ccccc1]

120. The process of embodiment 119, wherein the solvent or solvent mixture in (i) comprises one or more solvents selected from dichloromethane and ethyl acetate, preferably dichloromethane, or mixtures thereof.

121. The process of any of embodiments 119 or 120, wherein the solvent or solvent mixture in (i) comprises dichloromethane, preferably wherein the solvent in (i) is dichloromethane.

122. The process of any of embodiments 119 to 121, wherein providing a solution of the compound of formula (I″a) in a suitable solvent or solvent mixture in (i) comprises treating the compound of formula (I″a) in the solvent or solvent mixture with activated charcoal and/or silica gel, preferably with activated charcoal and silica gel and filtering the resulting mixture to obtain a clear solution.

123. The process of any of embodiments 119 to 122, wherein subjecting the solution of (i) to crystallization conditions in (ii) comprises adding a further solvent or solvent mixture.

124. The process of embodiment 123, wherein the further solvent or solvent mixture consists of or comprises pentane, hexane, heptane, diisopropyl ether, preferably heptane, or mixtures thereof.

125. The process of any of embodiments 123 or 124, wherein the further solvent or solvent mixture comprises heptane, preferably wherein the further solvent in (ii) is heptane.

126. The process of any of embodiments 123 to 125, wherein the further solvent or solvent mixture is added in a volume ratio of from 30:30 to 10:60, preferably of from 20:70 to 20:30, preferably of from 25:45 to 55:55 relative to the volume of the solvent or solvent mixture provided in (i).

127. The process of any of embodiments 119 to 126, wherein step (ii) comprises storing the mixture for a period of time in the range of from 1 to 72 hours, preferably of from 1 to 17 hours.

128. The process of any of embodiments 119 to 127, wherein step (ii) comprises storing the mixture at a temperature in the range of from 0 to 40° C., preferably in the range of from 20 to 30° C.

129. The process of any of embodiments 119 to 128, wherein step (ii) comprises storing the mixture for a period of time in the range of from 1 to 72 hours, preferably of from 1 to 17 hours at a temperature in the range of from 0 to 40° C., preferably in the range of from 20 to 30° C.

130. The process of any of embodiments 119 to 129, wherein step (ii) comprises seeding.

131. The process of any of embodiments 119 to 130, wherein (iii) comprises filtering, preferably filtering under vacuum, the resulting crystalline solid.

132. The process of any of embodiments 119 to 131, wherein (iii) comprises drying the resulting crystalline solid.

133. The process of embodiment 132, wherein (iii) comprises drying the resulting crystalline solid at a temperature of from 15 to 60° C., preferably of from 15 to 40° C., preferably of from 20 to 30° C., preferably of from 20 to 25° C., more preferably at 23° C. and at a pressure of from 5 to 100 mbar, preferably of from 15 to 80 mbar, preferably of from 20 to 50 mbar, more preferably of 30 mbar.

### Intermediates

134. A compound of formula (III)

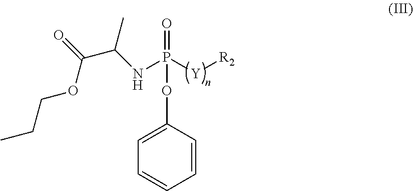

wherein (Y)ₙR₂ is a suitable leaving group for a nucleophilic substitution reaction.

135. The compound of embodiment 134, wherein n is 0 or 1 and wherein Y is O, N or S.

136. The compound of any of embodiments 134 or 135, wherein n is 1 and R₂ is alkyl, aryl, or heteroaryl, each optionally substituted with one or more electron-withdrawing groups, preferably aryl optionally substituted with one or more electron-withdrawing groups, more preferably phenyl optionally substituted with one or more electron-withdrawing groups.

137. The compound of any of embodiments 134 to 136 wherein n is 1 and R₂ is phenyl substituted with one or more electron-withdrawing groups, wherein the one or more electron-withdrawing groups are preferably F, Cl, Br, I, or NO₂.

138. The compound of any of embodiments 134 to 137, wherein n is 1, Y is O or S and R₂ is

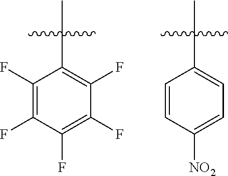 [SMILES: *c1c(F)c(F)c(F)c(F)c1F ; *c1ccc([N+](=O)[O-])cc1]

139. The compound of any of embodiments 134 to 138, wherein R₂ is

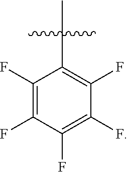 [SMILES: *c1c(F)c(F)c(F)c(F)c1F]

140. The compound of any of embodiments 134 or 135 wherein n is 1 and R₂ is a residue of formula (A)

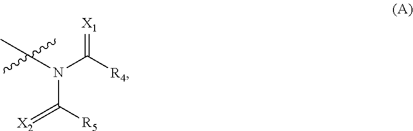

a residue of formula (B)

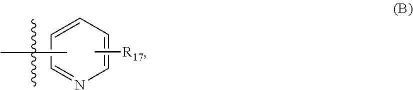

a residue of formula (C)

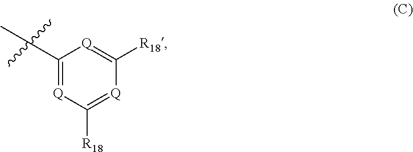

or a residue of formula (D)

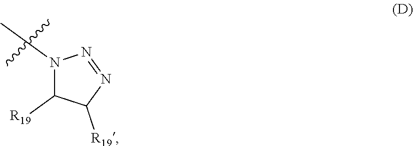

wherein at each occurrence

X₁ and X₂ are independently O or S;

R₄ and R₅ are independently H, OH, NH₂, C₁-C₆ alkyl or C₁-C₆ alkoxy, or

R₄ and R₅, together with the structure —C—N—C— according to formula (A), form an optionally substituted, 5-, 6-, or 7-membered saturated or partially unsaturated ring, wherein said ring is optionally fused to a 5- or 6-membered, optionally substituted ring which is a C₅-C₆ cycloalkyl, an aryl or a heterocycle comprising one or more heteroatoms independently being N, O or S;

R₁₇ is an electron-withdrawing group, preferably F, Cl, Br, I, NO₂, CHO, COOH, COO—(C₁-C₆)alkyl, CN, or COCl;

R₁₈ and R_{18′} are independently F, Cl, Br, I, or C₁-C₆ alkoxy;

each Q is independently C or N, wherein at least one Q is N;

R₁₉ and R_{19′} are independently H, OH, NH₂, C₁-C₆ alkyl optionally substituted with at least one of OH and NH₂, or C₁-C₆ alkoxy optionally substituted with at least one of OH and NH₂; or

R₁₉ and R_{19′} taken together form an optionally substituted 5-, 6-, or 7-membered saturated or partially unsaturated or aromatic ring, wherein the ring is optionally fused to a 5- or 6-membered, optionally substituted ring which is a C₅-C₆ cycloalkyl, an aryl, preferably benzo, or a heterocycle comprising one or more heteroatoms independently being N, O or S, the 5- or 6-membered optionally substituted ring preferably being heteroaryl.

141. The compound of any of embodiments 134 or 136, wherein n is 0 and R₂ is a residue of formula (A1)

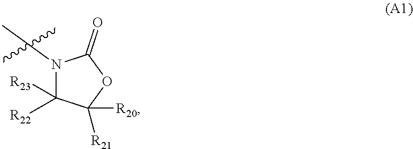

wherein R₂₀, R₂₁, R₂₂ and R₂₃ are each independently H, aryl, or C₁-C₆ alkyl optionally substituted with at least one of C₁-C₆ alkoxy optionally substituted with at least one of OH and NH₂; or

R₂₀ and R₂₂, or R₂₀ and R₂₃, or R₂₁ and R₂₂, or R₂₁ and R₂₃ when taken together form an optionally substituted 5-, 6-, or 7-membered saturated or partially unsaturated or aromatic ring which is an aryl, preferably benzo, or a heterocycle comprising one or more heteroatoms independently being N, O or S, the 5-, 6-, or 7-membered saturated or partially unsaturated or aromatic ring preferably being heteroaryl.

142. The compound of any of embodiments 134, 135 or 141, wherein the substituent of the optionally substituted 5-, 6-, or 7-membered saturated or partially unsaturated or aromatic ring which is an aryl, preferably benzo, or a heterocycle comprising one or more heteroatoms independently being N, O or S, is at least a substituent, preferably one substituent, selected from the group consisting of OH, C1-C6 alkoxy, aryl, heteroaryl, C3-C6 cycloalkyl, F, Cl, Br, I, COOH, CHO, C(O)(C1-C6 alkyl), C(O)(aryl), COO(C1-C6 alkyl), COONH2, COONH(C1-C6 alkyl), CN, NO2, —NH2, NR27R28, wherein R27 and R28 are independently selected from the group consisting of H, C1-C6 alkyl, C1-C6 alkoxy, aryl, heteroaryl, and wherein aryl at each occurrence is preferably phenyl.

143. The compound of any of embodiments 134, 135, 141 or 142, wherein the aromatic ring is a benzo substituted with at least one, preferably with one substituent, wherein the substituent is selected from the group consisting of OH, C1-C6 alkoxy, aryl, heteroaryl, C3-C6 cycloalkyl, F, Cl, Br, I, COOH, CHO, C(O)(C1-C6 alkyl), C(O)(aryl), COO(C1-C6 alkyl), COONH2, COONH(C1-C6 alkyl), CN, NO2, —NH2, NR27R28, wherein R27 and R28 are independently selected from the group consisting of H, C1-C6 alkyl, C1-C6 alkoxy, aryl, heteroaryl, and wherein aryl at each occurrence is preferably phenyl.

144. The compound of any of embodiments 141 to 143, wherein R22 and R23 are each independently H, aryl, or C1-C6 alkyl substituted with at least one of C1-C6 alkoxy optionally substituted with at least one of OH and NH2.

145. The compound of any of embodiments 134, 135 or 140, wherein n is 1 and R₂ is a residue of formula (A)

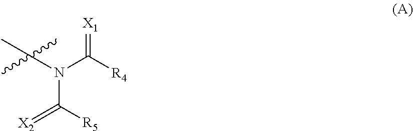

wherein

X₁ and X₂ are independently O or S;

R₄ and R₅ are independently H, OH, NH₂, C₁-C₆ alkyl or C₁-C₆ alkoxy, or

R₄ and R₅, together with the structure —C—N—C— according to formula (A), form an optionally substituted, 5-, 6-, or 7-membered saturated or partially unsaturated ring, wherein said ring is optionally fused to a 5- or 6-membered, optionally substituted ring which is a C₅-C₆ cycloalkyl, an aryl or a heterocycle comprising one or more heteroatoms independently being N, O or S.

146. The compound of any of embodiments 134, 135, 140 or 145, wherein R₂ is a residue of formula (IIb)

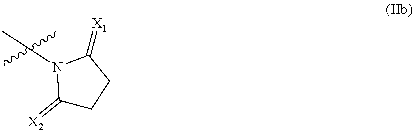

147. The compound of any of embodiments 134, 135, 140, 145 or 146, wherein R₂ is a residue of formula (IIc)

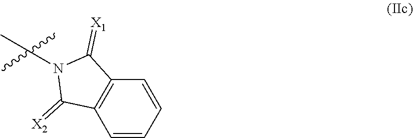

148. The compound of any of embodiments 134, 135, 140 or 145 to 147 wherein X1 is O and X2 is O.

149. The compound of any of embodiments 134, 135 or 140, wherein n is 1 and R₂ is a residue of formula (B)

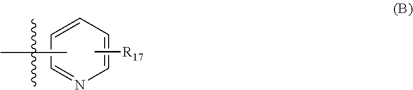

150. The compound of embodiment 149, wherein R17 is selected from the group consisting of F, Cl, Br, I, NO2, CHO, COOH, COO—(C1-C6)alkyl, CN and COCl.

151. The compound of any of embodiments 134, 135 or 138, wherein n is 1 and R₂ is a residue of formula (C)

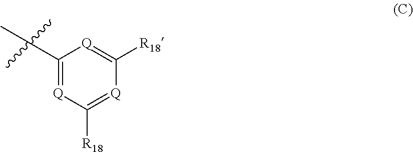

152. The compound of embodiment 151, wherein R18 and R18′ are independently F, Cl, Br, I, or C1-C6 alkoxy and each Q is independently C or N, wherein at least one Q is N.

153. The compound of embodiment 134, 135 or 140, wherein n is 1 and R₂ is a residue of formula (D)

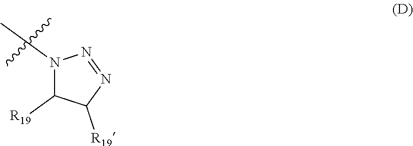

wherein R₁₉ and R₁₉′ are independently H, OH, NH₂, C₁-C₆ alkyl optionally substituted with at least one of OH and NH₂, or C₁-C₆ alkoxy optionally substituted with at least one of OH and NH₂; or

R₁₉ and R₁₉′ taken together form an optionally substituted 5-, 6-, or 7-membered saturated or partially unsaturated or aromatic ring, wherein the aromatic ring is preferably benzo,

wherein the ring is optionally fused to a 5- or 6-membered, optionally substituted ring which is a C₅-C₆ cycloalkyl, an aryl, preferably benzo, or a heterocycle comprising one or more heteroatoms independently being N, O or S, the 5- or 6-membered optionally substituted ring preferably being heteroaryl.

154. The compound of embodiment 153, wherein the substituent of the optionally substituted 5-, 6-, or 7-membered saturated or partially unsaturated or aromatic ring is at least a substituent, preferably one substituent, selected from the group consisting of OH, C1-C6 alkoxy, aryl, heteroaryl, C3-C6 cycloal-kyl, F, Cl, Br, I, COOH, CHO, C(O)(C1-C6 alkyl), C(O)(aryl), COO(C1-C6 alkyl), COONH2, COONH(C1-C6 alkyl), CN, NO2, —NH2, NR27R28, wherein R27 and R28 are independently selected from the group consisting of H, C1-C6 alkyl, C1-C6 alkoxy, aryl, heteroaryl, and wherein aryl at each occurrence is preferably phenyl.

155. The compound of any of embodiments 153 or 154, wherein the aromatic ring formed by R19 and R19′ taken together is a benzo substituted with at least one, preferably with one substituent, wherein the substituent is selected from the group consisting of OH, C1-C6 alkoxy, aryl, heteroaryl, C3-C6 cycloalkyl, F, Cl, Br, I, COOH, CHO, C(O)(C1-C6 alkyl), C(O)(aryl), COO(C1-C6 alkyl), COONH2, COONH(C1-C6 alkyl), CN, NO2, —NH2, NR27R28, wherein R27 and R28 are independent-ly selected from the group consisting of H, C1-C6 alkyl, C1-C6 alkoxy, aryl, heteroaryl, and wherein aryl at each occurrence is preferably phenyl.

156. The compound of any of embodiments 134, 135 or 145 to 148, wherein n is 1, Y is O and R2 is

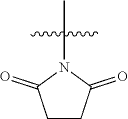 [SMILES: *N1C(=O)CCC1=O]

157. The compound of any of embodiments 134 or 135, wherein n is 0 and R2 is Cl.

158. The compound of any of embodiments 134 to 157, wherein the compound of formula (III) is the compound of formula (III′)

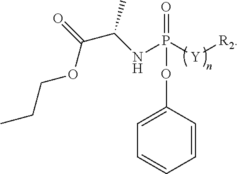

159. The compound of any of embodiments 134 to 158, wherein the compound of formula (III) is the compound of formula (III″) or the compound of formula (iii″), preferably the compound of formula (III″)

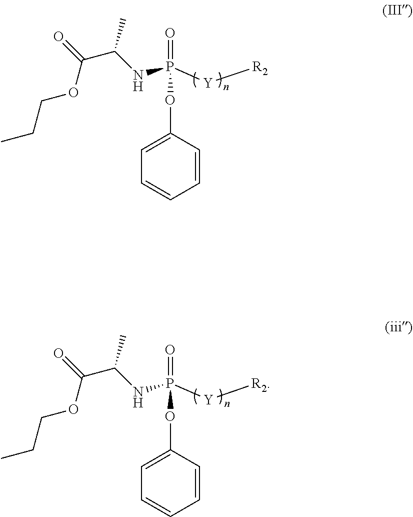

### Compositions

160. A composition comprising at least one compound of formula (I).

161. A composition comprising at least one compound of formula (I) according to any of embodiments 1 to 28.

162. The composition of embodiment 160, wherein the compound of formula (I) is the compound of formula (Ia), the compound of formula (I′a), the compound of formula (I″a) or the compound of formula (i″a), preferably the compound of formula (I″a).

163. The composition of any of embodiments 160 or 161 further comprising a pharmaceutically acceptable excipient.

164. The composition of any of embodiments 160 or 161 further comprising at least one pharmaceutically acceptable excipient.

165. The composition of any of embodiments 163 or 164, wherein the at least one pharmaceutically acceptable excipient is selected from the group consisting of carriers, fillers, diluents, lubricants, sweeteners, stabilizing agents, solubilizing agents, antioxidants and preservatives, flavouring agents, binders, colorants, osmotic agents, buffers, surfactants, disintegrants, granulating agents, coating materials and combinations thereof.

166. The composition of any of embodiments 163 to 166, wherein the at least one pharmaceutically acceptable excipient is selected from the group consisting of mannitol, microcrystalline cellulose, croscarmellose sodium, colloidal anhydrous silica and magnesium stearate.

165. The composition of any of embodiments 160 to 164 further comprising another antiviral agent.

166. The composition of embodiment 165 wherein the another antiviral agent is an NSSA inhibitor selected from the list consisting of Ledipasvir, Daclatasvir, Elbasvir, Odalasvir, Ombitasvir, Ravidasvir, Samatasvir, Ravidasvir and Velpatasvir, preferably wherein the another antiviral agent is Ledipasvir or Daclatasvir.

167. The composition of any of embodiments 165 or 166 wherein the another antiviral agent is Ledipasvir.

168. The composition of any of embodiments 165 or 166 wherein the another antiviral agent is Daclatasvir.

169. The composition of any of embodiments 165 or 166 wherein the another antiviral agent is Ravidasvir.

170. The composition of any of embodiments 160 to 169, wherein the compound of formula (I) according to any of embodiments 1 to 28 is present in an effective and/or predetermined amount.

171. The composition of embodiment 170, wherein the effective and/or predetermined amount is about 400 mg of the compound of formula (I), preferably 400 mg of the compound of formula (I).

172. The composition of any of embodiments 160 to 171, wherein the compound of formula (I) is present in an amount of from 25 to 60 weight-%, preferably of from 25 to 50 weight-%, preferably of from 30 to 45 weight-%, preferably of from 30 to 35 weight-%, more preferably about 33 weight-%, based on the total weight of the composition.

173. The composition of any of embodiments 160 to 172, wherein the compound of formula (I) is the compound of formula (I″a) according to embodiment 21.

### Uses

174. Use of a compound of formula (I) according to any of embodiments 1 to 28 or a composition according to any of embodiments 160 to 173 for the treatment of an infection in a human by a virus selected from HCV, West Nile virus, yellow fever virus, dengue virus, rhinovirus, polio virus, HAV, bovine viral diarrhea or Japanese encephalitis virus

175. Use according to embodiment 174, wherein the virus is HCV.

176. A compound of formula (I) according to any of embodiments 1 to 28 or a composition according to any of embodiments 160 to 173 for use in therapy.

177. A compound of formula (I) according to any of embodiments 1 to 28 or a composition according to any of embodiments 160 to 173 for use in the treatment of an infection in a human by a virus selected from HCV, West Nile virus, yellow fever virus, dengue virus, rhinovirus, polio virus, HAV, bovine viral diarrhea or Japanese encephalitis virus

178. The use of any of embodiments 174 or 175 or the compound or composition for use according to any of embodiments 176 or 177 wherein the virus is HCV.

179. The use of any of embodiments 174, 175 or 178 or the compound or composition for use according to any of embodiments 176 or 177 wherein the compound of formula (I) is the compound of formula (I″a) or the compound of formula (i″a), preferably the compound of formula (I″a)

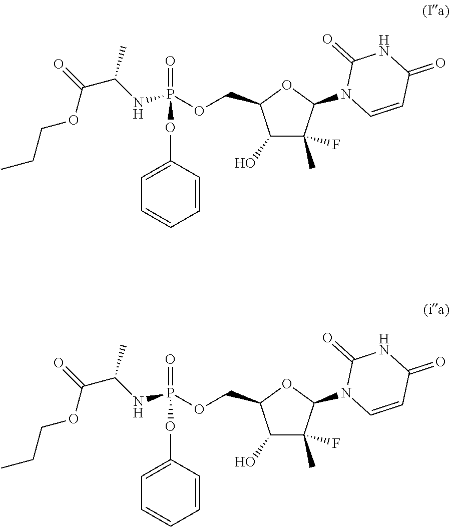 [SMILES: CCCOC(=O)[C@H](C)N[P@](=O)(OC[C@H]1O[C@@H](n2ccc(=O)[nH]c2=O)[C@](C)(F)[C@@H]1O)Oc1ccccc1 ; CCCOC(=O)[C@H](C)N[P@@](=O)(OC[C@H]1O[C@@H](n2ccc(=O)[nH]c2=O)[C@](C)(F)[C@@H]1O)Oc1ccccc1]

180. The use of any of embodiments 174, 175 or 178 to 179 or the compound or composition for use according to any of embodiments 176, 177 or 178 to 179 wherein the use further comprises administering to the subject an effective amount of another antiviral agent.

181. The use of embodiment 180 wherein the another antiviral agent is an NS5A inhibitor, preferably an NS5A inhibitor selected from the list consisting of Ledipasvir, Daclatasvir, Elbasvir, Odalasvir, Ombitasvir, Ravidasvir, Samatasvir, Ravidasvir and Velpatasvir, preferably wherein the another antiviral agent is Ledipasvir or Daclatasvir.

182. The use of any of embodiments 180 or 181 wherein the another antiviral agent is Ledipasvir.

183. The use of any of embodiments 180 or 181 wherein the another antiviral agent is Daclatasvir.

184. The use of any of embodiments 180 or 181 wherein the another antiviral agent is Ravidasvir.

### Methods of Treatment

185. A method of treating a human infected by hepatitis C virus comprising administering to the subject an effective amount of a compound of formula (I), a compound of formula (Ia), a compound of formula (I′), a compound of formula (I′a), a compound of formula (I″), a compound of formula (I″a) or a compound of formula (i″a), preferably a compound of formula (I″a) according to any of embodiments 1 to 28 or a composition comprising of a compound of formula (I), a compound of formula (Ia), a compound of formula (I′), a compound of formula (I′a), a compound of formula (I″), a compound of formula (I″a) or a compound of formula (i″a), preferably a compound of formula (I″a) according to embodiment 21.

186. The method of embodiment 185, wherein the method comprises administering the compound or the composition to the human once, twice, three times or four times daily, preferably once daily.

187. The method of any of embodiments 185 or 186, wherein the method comprises administering the compound or the composition to the human in a tablet or a capsule form, preferably in a tablet form.

188. The method of any of embodiments 185 to 187, wherein the human is infected with hepatitis C virus genotype 1, 2, 3, 4, 5 or 6 or a combination thereof.

### Experimental

Experimental Conditions

X-ray powder diffraction patterns (XRPD, PXRD) were obtained with a PANalytical X'Pert PRO diffractometer equipped with a theta/theta coupled goniometer in transmission geometry, Cu-Kα1.2 radiation (wavelength 0.15419 nm) with a focusing mirror and a solid state PIXcel detector. The patterns were recorded at a tube voltage of 45 kV and a tube current of 40 mA, applying a 2-theta step size of 0.013° with 40 s per step (255 channels) in the 2-theta angular range of 2° to 40° at ambient conditions.

Gravimetric Moisture Sorption: Moisture sorption isotherms were recorded with an SPSx-1 μ moisture sorption analyzer (ProUmid, Ulm). The measurement cycle was started at ambient relative humidity (r.h.) of 35%. Relative humidity was then decreased to 5% r.h. in 5% steps, followed by a further decrease to 3% r.h. and to 0% r.h. Afterwards r.h. was increased from 0% to 95% r.h. in a sorption cycle and decreased to 0% in a desorption cycle in 5% steps. Finally r.h. was increased to 35% r.h. in 5% steps. The time per step was set to a minimum of 2 hours and a maximum of 6 hours. If an equilibrium condition with a constant mass of ±0.01% within 1 hour was reached before the maximum time for all examined samples the sequential humidity step was applied before the maximum time of 6 hours. If no equilibrium was achieved the consecutive humidity step was applied after the maximum time of 6 hours. The temperature was 25±0.1° C.

NMR spectra were recorded on a Bruker AVANCE III HD 400 nano spectrometer equipped with a Prodigy Cryoprobe head. ¹H and ¹³C spectra were recorded in DMSO-d6 at 298 K. Chemical shifts are reported as δ-values in ppm relative to the residual solvent peak of DMSO-d6 (δ_{H}: 2.50; δ_{C}: 39.5). For the characterization of the observed signal multiplicities the following abbreviations were used: s (singlet), d (doublet), t (triplet), q (quartet), quint (quintet), sept (septet), m (multiplet) as well as br (broad).

### Synthesis

Example 1. Preparation of Compound 1″a (n-Propyl-Sofosbuvir)

Example 1.1 (Step 1) Preparation of Propyl ((2,5-dioxopyrrolidin-1-yl)oxy)(phenoxy)phosphoryl)-L-alaninate

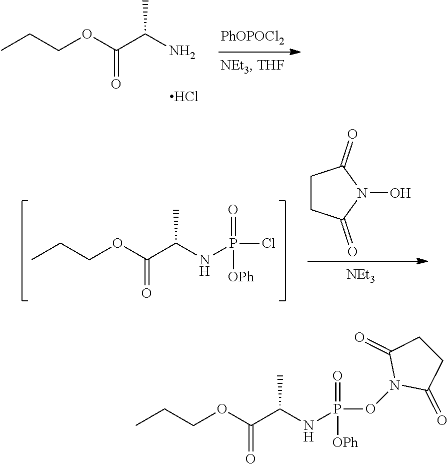

Propyl-L-alaninate hydrochloride (30.0 g, 179 mmol) was dissolved in THF (390 mL). Molecular sieves (4 Å, 16.5 g) and phenyl phosphorodichloridate (25.1 mL, 167 mmol) were added. The reaction was cooled to 5° C. and triethylamine (48.7 mL, 351 mmol) was added over 10 min. The colorless supension was stirred for 20 min at 5° C. N-Hydroxysuccinimide (18.7 g, 161 mmol) was added and triethylamine (24.3 mL, 175 mmol) was added over 10 min, while the temperature did not exceed 5° C. After 20 min the reaction was filtered and the filtrate was concentrated and redissolved in MTBE (99 mL). The solution was added to MTBE (900 mL) at 30° C. and seed crystals (100 mg) were added at 25° C., before it was cooled to −10° C. and stirred at this temperature for 16 h. The formed precipitate was filtered and dried to give the desired product as a colorless solid (12.0 g, 17%, dr 4:1).

1H NMR (DMSO, 300 MHz): δ/ppm 7.42-7.37 (m, 2H), 7.26-7.22 (m, 3H), 6.75 (dd, J=15.0, 10.0 Hz, NH), 4.14-4.05 (m, 1H), 4.00 (t, J=6.5 Hz, 2H), 2.71 (s, 4H), 1.58 (sextett, J=7.0 Hz, 2H), 1.31 (d, J=7.0 Hz, 3H), 0.87 (t, J=7 Hz, 3H).

13C NMR (DMSO, 75 MHz): δ/ppm 172.78, 170.23, 150.29, 150.19, 129.74, 125.16, 120.00, 66.09, 49.89, 25.39, 21.49, 10.19.

31P NMR (DMSO, 121 MHz): δ/ppm 5.28 (20%), 4.33 (80%).

Example 1.2 (Step 2) Preparation of n-Propyl ((S)-(((2R,3R,4R,5R)-5-(2,4-dioxo-3,4-dihydropyrimidin-1(2H)-yl)-4-fluoro-3-hydroxy-4-methyltetrahydrofuran-2-yl)methoxy)(phenoxy)phosphoryl)-L-alaninate (Compound I″a), (n-Propyl-Sofosbuvir)

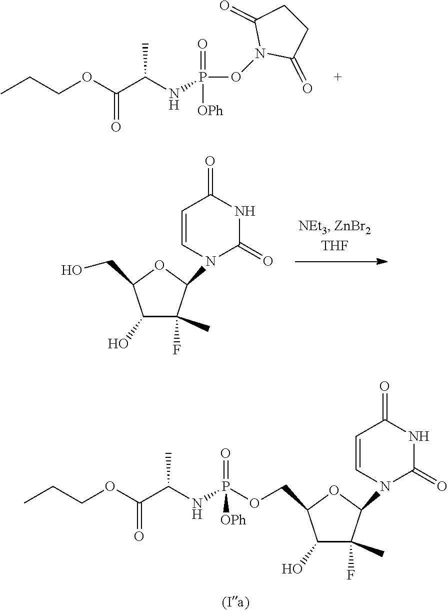 [SMILES: CCCOC(=O)[C@H](C)N[P@@](=O)(Oc1ccccc1)ON1C(=O)CCC1=O ; C[C@@]1(F)[C@H](O)[C@@H](CO)O[C@H]1n1ccc(=O)[nH]c1=O ; CCCOC(=O)[C@H](C)N[P@](=O)(OC[C@H]1O[C@@H](n2ccc(=O)[nH]c2=O)[C@](C)(F)[C@@H]1O)Oc1ccccc1]

1-((2R,3R,4R,5R)-3-Fluoro-4-hydroxy-5-(hydroxymethyl)-3-methyltetrahydrofuran-2-yl) pyrimidine 2,4(1H,3H)-dione (1.88 g, 7.22 mmol) was dissolved in THF (56 mL) while heating. At 22° C. propyl (((2,5-dioxopyrrolidin-1-yl)oxy)(phenoxy)phosphoryl)-L-alaninate (5.00 g, 13.0 mmol), 4 Å molecular sieves (2.55 g) and ZnBr2 (1.63 g, 7.22 mmol) were added. After 10 min NEt3 (2.00 mL, 14.5 mmol) was added. The reaction was stirred at 22° C. for 5 h before it was filtered. The residue was washed with THF (5 mL) and water (22 mL) was added to the filtrate. The biphasic filtrate was concentrated under reduced pressure to remove organic solvents. CH2Cl2 (22 mL) was added to the residue and heated to give a clear biphasic solution. HCl (2.5 M, 6 mL) was added and the layers were separated. Sodium acetate (15% in water, 22 mL) was added to the organic layer and heated to 35° C. The layers were separated. Water (22 mL) was added to the organic layer and heated to 35° C. The layers were separated and the organic layer was concentrated under reduced pressure to give a colorless foam (2.37 g, 62%).

1H NMR (DMSO, 300 MHz): δ/ppm 11.3 (br s, NH), 7.57 (d, J=8 Hz, 1H), 7.40-7.35 (m, 2H), 7.24-7.16 (m, 3H), 6.14-5.53 (m, 3H), 5.55 (d, J=8 Hz, 1H), 4.37 (dd, J=12, 6 Hz, 1H), 4.25 (dd, J=12, 6 Hz, 1H), 4.03-3.82 (m, 5H), 1.55 (sexett, J=7 Hz, 2H), 1.29-1.21 (m, 6H), 0.85 (t, J=7 Hz, 3H).

13C NMR (DMSO, 75 MHz): δ/ppm 173.19, 162.77, 150.73, 150.65, 150.45, 129.68, 124.61, 120.11, 102.26, 101-51, 99.11, 79.42, 71.6, 65.97, 64.77, 49.75, 21.46, 19.91, 16.40 (d), 10.14.

31P NMR (DMSO, 121 MHz): δ/ppm 3.76 (91%), 3.67 (9%).

Example 2. Alternative Preparation of Compound I″a (n-Propyl-Sofosbuvir)

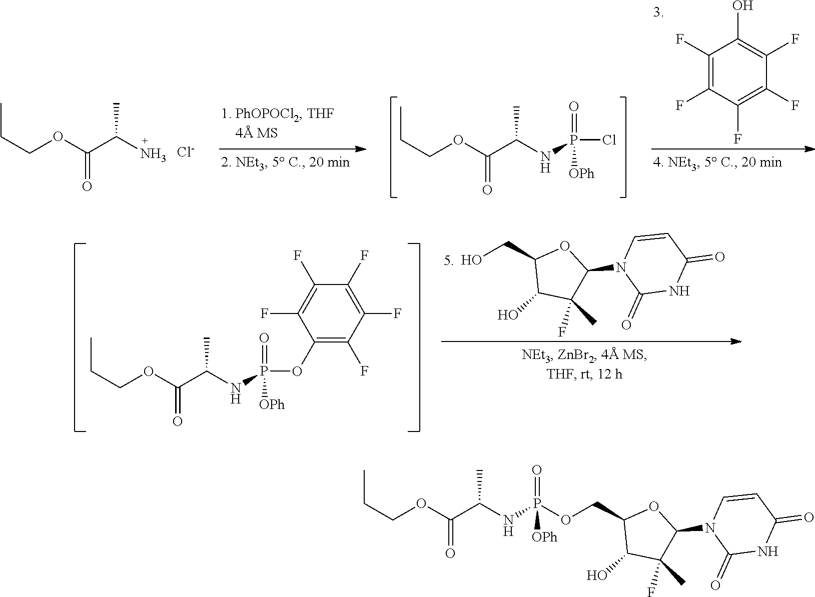

Step 1: In a 1.0 L round bottom flask, equipped with a magnetic stirrer and an inlet temperature sensor, propyl-L-alaninate (30.0 g, 179 mmol, 1.11 equiv) was dissolved in THF (390 mL). Molecular sieves (16.5 g, 4 Å) and phosphorodichloridate (25.1 mL, 167 mmol, 1.04 equiv) were added and the mixture was cooled to 5° C. Then, triethylamine (48.7 mL, 351 mmol, 2.18 equiv) was added dropwise over 30 min and the resulting suspension was stirred for another 20 min at 5° C. Next, pentafluorophenol (29.6 g, 161 mmol, 1.00 equiv) was added, followed by the dropwise addition of triethylamine (24.3 mL, 175 mmol, 1.09 equiv) over 20 min and stirring was continued for 20 min at 5° C. The reaction mixture was filtered to remove all solids and the clear solution was used without further purification in the next step.

Step 2: In a 500 mL round bottom flask, equipped with a magnetic stirrer and an inlet temperature sensor, 1-((2R,3R,4R,5R)-3-fluoro-4-hydroxy-5-(hydroxymethyl)-3-methyltetrahydrofuran-2-yl)pyrimidine-2,4(1H,3H)-dione (6.25 g, 24.0 mmol, 1.0 equiv) was dissolved in 155 mL THF while heating. Molecular sieves (6.00 g, 4 Å) and ZnBr₂ (5.65 g, 25.1 mmol, 1.05 equiv) were added at 22° C. before 130 mL of the in before prepared solution of propyl ((S)-(perfluorophenoxy)(phenoxy)phosphoryl)-L-alaninate (in theory: 12.8 g, 28.2 mmol, 1.18 equiv) was slowly transferred to the suspension and stirring was continued for 10 min. Triethylamine (6.02 mL, 43.4 mmol, 1.81 equiv) was added slowly and the reaction mixture was stirred at 22° C. for 12 h. After filtration, in order to remove all solids, 44 mL deionized water was added and the biphasic filtrate was concentrated under reduced pressure to remove all organic solvents. Then, CH₂Cl₂ (50 mL) and HCl (2.5 M, 12 mL) were added to the residue and the layers were separated. The organic phase was washed with NaOAc (15% in water, 44 mL) at 35° C. for 5 min and the layers were again separated. The organic phase was extracted with water (44 mL) for 10 min at 35° C. and dried over Na₂SO₄ (15 g) and activated charcoal (5.0 g). After filtration, the solvents were removed under reduced pressure to yield 18.1 g of the crude product. This residue was dissolved in CH₂Cl₂ (200 mL) and heptane was added until the solution became turbid (after appr. 200 mL). After the mixture was stirred for 1 h at 22° C., another 100 mL heptane was added and crystallization started. After 3 h stirring at 22° C., the suspension was filtered and dried under vacuum to yield 6.95 g of I″a (13.1 mmol, 55%, 85.6% pure by NMR).

Example 3. Alternative Process for the Preparation of Compound I″a (n-Propyl-Sofosbuvir)

Example 3.1 (Step 1): Preparation of Propyl ((S)-(perfluorophenoxy)(phenoxy)phosphoryl)-L-alaninate:

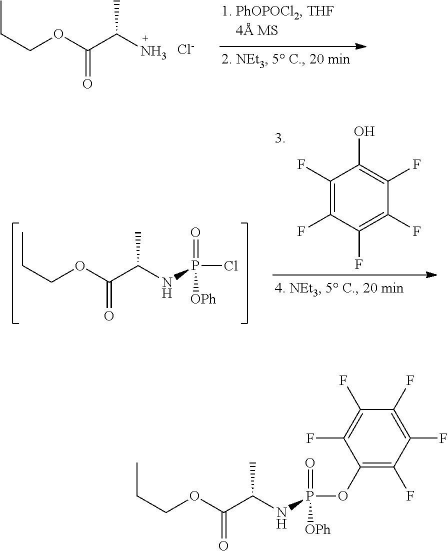

In a 1.0 L round bottom flask, equipped with a magnetic stirrer and an inlet temperature sensor, propyl-L-alaninate (30.0 g, 179 mmol, 1.11 equiv) was dissolved in THF (390 mL). Molecular sieves (16.5 g, 4 Å) and phosphorodichloridate (25.1 mL, 167 mmol, 1.04 equiv) were added and the mixture was cooled to 5° C. Then, triethylamine (48.7 mL, 351 mmol, 2.18 equiv) was added dropwise over 30 min and the resulting suspension was stirred for another 20 min at 5° C. Next, pentafluorophenol (29.6 g, 161 mmol, 1.00 equiv) was added, followed by the dropwise addition of triethylamine (24.3 mL, 175 mmol, 1.09 equiv) over 20 min and stirring was continued for 20 min at 5° C. The reaction mixture was filtered to remove all solids and washed with THF, resulting in 825 mL of a clear solution. With 695 mL (app. 84% of the reaction mixture) was continued as followed: The solution was concentrated under reduced pressure and 100 mL MTBE was added. The mixture was stirred at 0° C. for 2 h and crystallization started. The solid was collected by filtration and dried under vacuum to yield 37.8 g of the desired product (HPLC: 97.3% of the total area). ¹H NMR (400 MHz, DMSO) δ/ppm=7.42 (t, J=7.9 Hz, 2H), 7.28-7.22 (m, 3H), 6.93-6.86 (m, 1H), 4.02-3.95 (m, 3H), 1.55 (sext, J=7.1 Hz, 2H), 1.30 (t, J=5.6 Hz, 3H), 0.86 (t, J=7.4 Hz, 3H).

¹³C NMR (101 MHz, DMSO) δ/ppm=173.04, 150.49, 142.70, 140.21, 138.27 (d, J=178.9 Hz), 136.68, 130.32, 125.83, 120.48, 66.56, 50.52, 21.89, 20.04, 10.54.

³¹P NMR (162 MHz, DMSO) δ/ppm=0.32 (s).

Example 3.2 (Step 2): Preparation of Compound I″a (n-Propyl-Sofosbuvir)

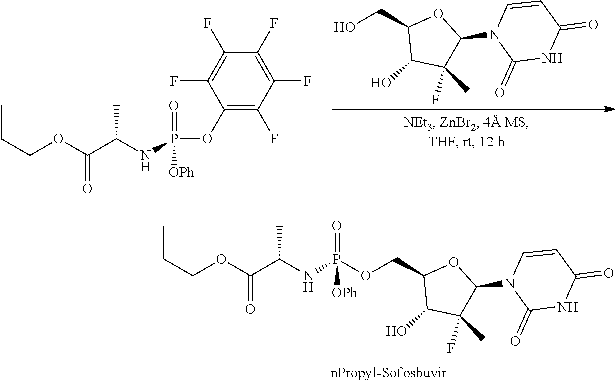 [SMILES: CCCOC(=O)[C@H](C)N[P@](=O)(Oc1ccccc1)Oc1c(F)c(F)c(F)c(F)c1F ; C[C@@]1(F)[C@H](O)[C@@H](CO)O[C@H]1n1ccc(=O)[nH]c1=O ; CCCOC(=O)[C@H](C)N[P@](=O)(OC[C@H]1O[C@@H](n2ccc(=O)[nH]c2=O)[C@](C)(F)[C@@H]1O)Oc1ccccc1]

In a 1.0 L round bottom flask, equipped with a magnetic stirrer and an inlet temperature sensor, 1-((2R,3R,4R,5R)-3-fluoro-4-hydroxy-5-(hydroxymethyl)-3-methyltetrahydrofuran-2-yl)pyrimidine-2,4(1H,3H)-dione (21.9 g, 84.2 mmol, 1.09 equiv) was dissolved in 648 mL THF while heating. Molecular sieves (21.0 g, 4 Å), ZnBr₂ (19.8 g, 88.0 mmol, 1.14 equiv) and propyl ((S)-(perfluorophenoxy)(phenoxy)phosphoryl)-L-alaninate (35.0 g, 77.2 mmol, 1.00 equiv) were added and stirring was continued for 10 min. Triethylamine (20.98 mL, 151 mmol, 1.96 equiv) was added slowly and the reaction mixture was stirred at 22° C. for 21 h. After filtration, in order to remove all solids, 154 mL deionized water was added and the biphasic filtrate was concentrated under reduced pressure to remove all organic solvents. Then, CH₂Cl₂ (175 mL) and HCl (2.5 M, 42 mL) were added to the residue and the layers were separated. The organic phase was washed with NaOAc (15% in water, 154 mL) at 35° C. for 5 min and the layers were again separated. The organic phase was extracted with water (154 mL) for 10 min at 35° C. and dried over Na₂SO₄ (52.5 g) and activated charcoal (17.5 g). After filtration, the solvents were removed under reduced pressure to obtain crude I″a. This was dissolved in CH₂Cl₂ (700 mL). Then, heptane was added until the solution became turbid (after appr. 350 mL) and seeds were added. After the mixture was stirred for 1 h at 22° C., another 350 mL heptane was added and the mixture was stirred for 3 h at 22° C. The precipitate was collected by filtration and dried under vacuum to yield 19.98 g of I″a (37.7 mmol, 48%, 98.9% pure by NMR).

¹H NMR (400 MHz, DMSO) δ/ppm=11.53 (br s, 1H), 7.57 (d, J=7.9 Hz, 1H), 7.38 (t, J=7.9 Hz, 2H), 7.24-7.17 (m, 3H), 6.09 (q, J=7.7 Hz, 2H), 5.86 (d, J=6.0 Hz, 1H), 5.55 (d, J=8.3 Hz, 1H), 4.39-4.21 (m, 2H), 4.03-2.83 (m, 5H), 1.55 (sext, J=7.0 Hz, 2H), 1.27 (d, J=8.6 Hz, 3H), 1.24 (d, J=6.9 Hz, 3H), 0.86 (t, J=7.4 Hz, 3H).

¹³C NMR (101 MHz, DMSO) δ/ppm=173.61 (d, J=5.1 Hz), 163.21, 151.17, 151.11, 150.90, 130.13, 125.06, 120.52 (d, J=4.9 Hz), 102.72, 100.76 (d, J=180.3 Hz), 79.96, 71.89, 66.42, 65.19, 55.38, 50.19, 21.90, 20.32 (d, J=6.5 Hz), 17.00 (d, J=25.4 Hz), 10.59.

31P NMR (162 MHz, DMSO) δ/ppm=3.76 (92%), 3.67 (8%).

Example 4. Preparation of Seeding Material of Compound (I″a) (n-Propyl-Sofosbuvir)

100 mg of amorphous (I″a) (crude) were mixed with 35 mg activated charcoal and 40 mg of silica gel in 2 ml dichloromethane. After stirring for 5 minutes, the mixture was filtered through a syringe filter and the obtained clear solution was diluted with heptane (0.7 ml) until a turbidity was obtained. The mixture was stored at room temperature for several days to obtain a precipitate. This suspension was stored in a fridge to use it for seeding.

Example 5. Preparation of Crystalline Compound (I″a) (n-Propyl-Sofosbuvir)

2.30 g of amorphous (I″a) (crude) were mixed with 0.81 g activated charcoal and 0.91 g of silica gel in 46 ml dichloromethane. After stirring for 5 minutes, the mixture was filtered and the obtained clear solution was diluted with heptane (18 ml) until a turbidity was obtained. Seeds added and the mixture was stored at room temperature for four days. The resulting precipitate was isolated and dried to yield 620 mg of crystalline (I″a).

Example 6. Alternative Preparation of Crystalline Compound (I″a) (n-Propyl-Sofosbuvir)

3.92 g of crude II″a prepared according to Example 3.2 above was dissolved in 76 mL dichloromethane at 22° C. To this, heptane was slowly added under stirring, until the solution become turbid (appr. after 46 mL heptane) and seeds were added. After 1 h, another 30 mL heptane were added to the suspension and stirring was continued for 3 h. The product was collected by filtration and dried under high vacuum to yield 2.18 g crystalline I″a (55%, 97.19% pure by NMR, dr=98:2).

Example 7. Transformation of Crystalline Compound I″a (n-Propyl-Sofosbuvir) to Amorphous Material

In a 20 mL glass flask, equipped with a mechanical stirrer, 200 mg of crystalline compound I″a prepared as described according to example 3.2 above was suspended in 6 mL solvent (table 1) and stirred (260 rpm) at 37° C. for 24 h. A sample was then taken and analyzed by XRPD to confirm the formation of amorphous material. In all cases, only amorphous material and no remaining trace of crystalline material was detected.

TABLE 1
solvents tested for the preparation of amorphous I″a

| Entry | Solvent | Resulting material |
|---|---|---|
| 1 | HCl (pH = 2.03) | amorphous |
| 2 | Water (pH = 7.84) | amorphous |
| 3 | Acetate buffer (pH = 4.63) | amorphous |
| 4 | Phosphate buffer (pH = 6.89) | amorphous |
| 5 | HCl (pH = 1.25) | amorphous |

### Activity Analysis for Compound I″a (n-Propyl-Sofosbuvir)

Materials & Methods

Production of HC-Virus stock. HCV-Jc1/Ypet plasmids are linearized by Xbal and purified with the Wizard SV gel and PCR clean-up system (Promega). Purified template DNA (1 μg) is subsequently transcribed using the MEGAscript T7 RNA production system (Ambion). Template DNA is removed by treatment with Turbo DNase (Ambion) at 37° C. for 15 min. RNA is cleaned up by an RNeasy minikit (Qiagen), and RNA quality is monitored by agarose gel electrophoresis. RNA (10 μg) is electroporated into 5×10⁶ Huh-7.5.1 cells using 4-mm gap electroporation cuvettes (Fisher Scientific). After one pulse at 950 μF and 270 V with a Gene Pulser system II (Bio-Rad), cells are suspended in DMEM plus 10% FBS and plated in a T175 flask.

Polyethylene glycol (PEG) precipitation of extracellular HCV particles. Virus-containing culture supernatants are clarified by centrifugation (3,000×g) and transferred to 15-ml disposable conical centrifuge tubes. Viruses are precipitated by adding one-fourth volume sterile-filtered 40% (wt/vol) PEG-8000 in phosphate-buffered saline (PBS) (final concentration of 8% [wt/vol]) and overnight incubation at 4° C. Viral precipitates are collected by centrifugation (4,000×g, 30 min) and washed twice with PBS. Supernatants were removed, and pellets were resuspended in 1 ml of DM EM containing 10% FBS.

Limited dilution assay (TCID₅₀). A total of 6×10³ cells/well were plated onto a 96-well plate. The cells were infected with 50 μl of six serial dilutions ranging from undiluted to 10⁻⁵; 72-h postinfection (hpi) cells were fixed with 100% methanol for 30 min at −20° C. and then washed with PBS followed by 0.1% Tween 20 in PBS (PBS-T). The cells were permeabilized with PBS-T and blocked with 1% bovine serum albumin (BSA)-0.2% skim milk in PBS-T. Hydrogen peroxide (3%) was added to block the endogenous peroxidase activity. The cells were stained with mouse monoclonal primary NSSA antibody 9E10 (1:25,000), ImmPRESS anti-mouse IgG (1:3) (Vector Laboratories), and 3,3′-diaminobenzidine (DAB) substrate (1 drop/ml) (Invitrogen), respectively. The NSSA-positive wells were counted and recorded by using a light microscope. The 50% tissue culture infectious dose (TCID₅₀) was calculated by a Reed-Muench calculator as previously described.

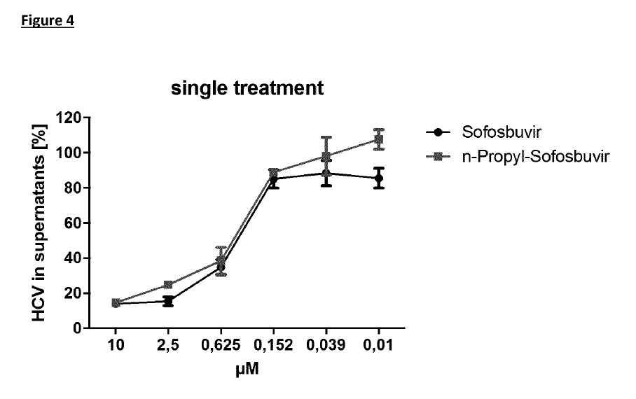

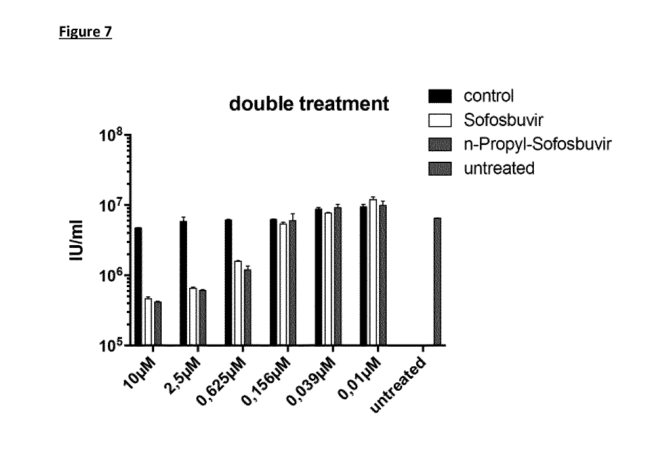

HCV histochemistry and determination of virus titers. To determine virus titers, the protocol described by Linden bach et al. (2005) is slightly modified. 1×10⁴ Huh-7.5 cells or 0.7×10⁴ Lunet cells were seeded per 96-well 24 h prior to infection. Six wells are infected simultaneously with the same dilution of filtered cell culture supernatants of HCV transfected or infected cells. Usually, the first dilution is a 1:10 dilution followed by 1:6 dilutions. 72 h after infection, the cells are washed once with PBS and then fixed in ice-cold methanol for 20 min at −20° C. Afterwards, the cells are washed with PBS and then permeabilized with 0.5% Triton X-100 in PBS for 5 min at RT. The first antibody detecting the HCV NSSA protein (9E10) is diluted 1:2000 in PBS and was incubated on the cells for 1 h. Then the cells are washed again three times with PBS and stained with the secondary antibody (goat a-mouse coupled to HRP, Sigma) 1:200 in PBS for 45 min at RT. To detect the HCV positive cells, the wells are first washed again three times with PBS and then the HRP activity is detected by the addition of 30 μl Carbazole substrate/96-well for 15 min at RT. Afterwards, the substrate is replaced with water and the wells were analyzed by light microscopy for positive cells. The 50% tissue culture infectious dose (TCID₅₀) is calculated based on the methods described by Spearman and Karber. By this, the concentrations of a virus isolation needed to infect 50% of a given number of wells is determined (Spearman, 1908). qPCR of viral supernatant. Total RNA was extracted by the Altostar system according to the protocol of the manufacturer. A Power SYBR Green RNA-to-Ct 1-step kit (Applied Biosystems) is used to quantify the amount of HCV RNA. Primers specific for the 5′ UTR are 5′-TGCGGAACCGGTGAGTACA-3′ (forward) and 5′-TGCGGAACCGGTGAGTACA-3′ (reverse). The PCR program conditions are as follows: 30 min at 48° C. for reverse transcription, 10 min at 95° C. for enzyme activation, and 40 cycles of amplification with 15 s at 95° C. for denaturation and 1 min at 60° C. for annealing and extension. Standard curve reactions are run in parallel by using serially diluted Jc1/Gluc2A plasmid ranging from 2.0×10⁷ to 2.0×10⁰ copies. To confirm obtained for experiment 3 (FIGS. 4 and 7), PCR was performed using the Atlona HCV-quantification kit.

Efficacy Testing of Sofosbuvir and n-Propyl-Sofosbuvir (compound I″a). Huh7.5 cells (1×10⁴ per well) were seeded in a 96-well plate. The following day cells were infected with 8500 TCID₅₀/well of HCV (Jc-1 Wild-type virus) for infection. After 48 h cells were treated with Sofosbuvir, n-Propyl-Sofosbuvir and as a negative control with the solvent used for Sofosbuvir, n-Propyl-Sofosbuvir (DMSO/EtOH) at concentrations given in the Figures. Two days or three later, supernatants were harvested and RNA was extracted by the Altostar system according to the protocol of the manufacturer. Quantitative PCR was performed using the Atlona HCV-quantification kit as recommended by the manufacturer.

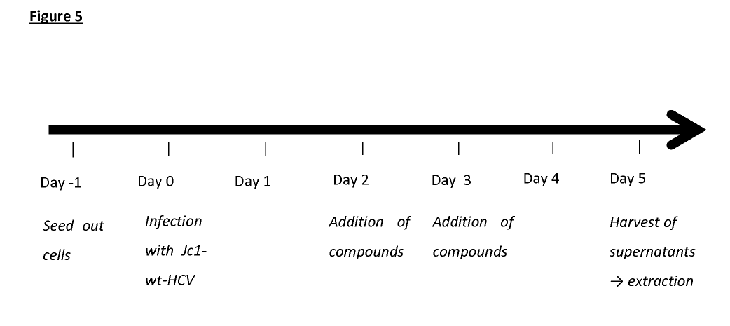

In one set of experiments, Sofosbuvir and n-Propyl-Sofosbuvir were applied 48 h post infection and again 24 h later as indicated by FIG. 5.

All experiments shown represent the mean of at least two independent sets of data performed in duplicates.

Results:

Evaluation of the Effective Dose of the Drugs

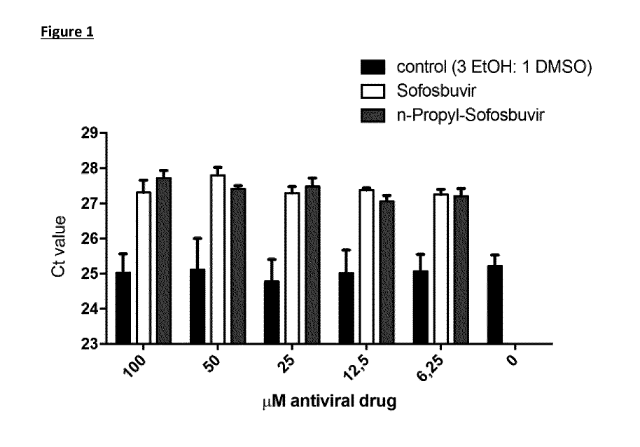

48 h post infection, cells were incubated with decreasing amounts the compounds in the μm range as given in the Figure. Two days later, RNA was extracted and HCV-RNA was amplified by SYBR Green RNA-to-Ct 1-step kit and the cₜ-values, obtained are shown in FIG. 1 (y-axis). To exclude any toxic effect of the solvent on the infection, the amount of EtOH/DMSO in the wells of the control was equal as applied in the Sofosbuvir and n-Propyl-Sofosbuvir group. As expected, the untreated control group was unable to reduce the amount of HCV-RNA independent on the presence of EtOH/DMSO, the Ct-value of all samples was around a threshold cycle of 25. In contrast, both Sofosbuvir and n-Propyl-Sofosbuvir inhibited virus production equally well even in the lowest doses used in the assay. The Cₜ-value was around 28 indicating that about 1 log virus reduction was achieved. The nearly similar efficacy of the compounds was a not expected as according to the literature, the IC₅₀ of Sofosbuvir is clearly above 10 μM after 48 h of infection (Liu et al, Antimicrob. Agents Chemother. 2015) and more efficient as compared to replicon systems (Sofia et al, J. Med Chem, 2010). This discrepancy is most likely due to the different read outs used. In contrast to Liu et al., which used HCV-Jc-1 which is tagged to a yellow fluorescent protein, the virus used in our assay resembles an unmodified wild-type HCV strain.

Single Application of the Drugs

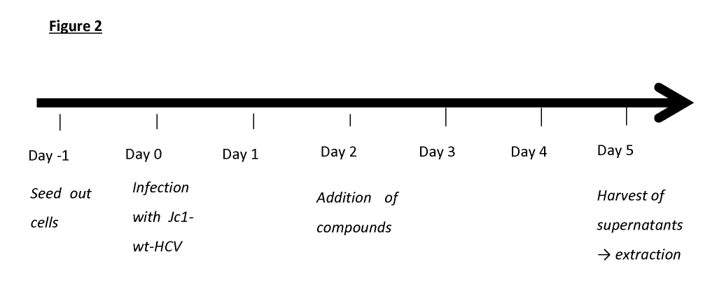

In a next set of experiments, we introduced the following changes: The infection period was extended to 72 h (see FIG. 2) and the concentration range of the compounds were further increased down to 9 nM to check, if the effect of the drugs is dose-dependent.

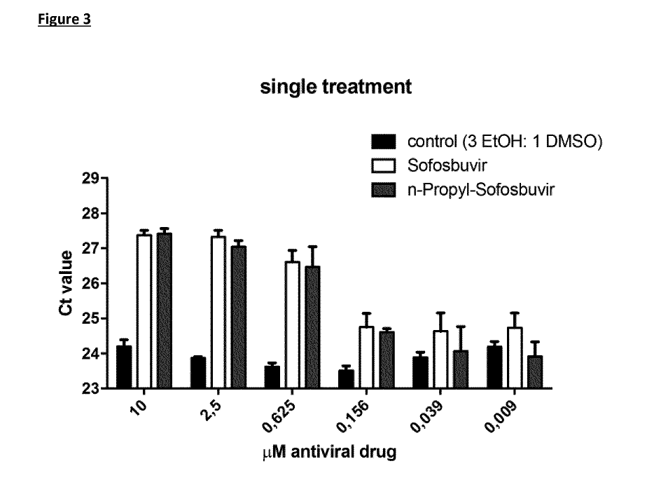

As shown in FIG. 3, the increased incubation time to 72 h was parallel to an increase in the viral RNA, which is reflected by a decrease in the threshold cycle of the untreated controls from 25 (in FIG. 1) to 24 in the actual FIG. 3.

Using the HCV quantification Kit from Altona, we further tested for the efficacy of Sofosbuvir and n-Propyl-Sofosbuvir. The results indicate that both compounds have a comparable antiviral profile with probably slight advantages of Propyl-Sofosbuvir in the lowest concentration range used (FIG. 4).

Two-Time Application of the Drugs

Next experiments yielded in the evaluation of the drugs when Sofosbuvir and n-Propyl-Sofosbuvir were given twice as indicated by the schematic overview in FIG. 5.

Again, the extended incubation time of the cells with HCV increase the amount of viral RNA to a cₜ value of around 23. In the presence of Sofosbuvir and n-Propyl-Sofosbuvir, the viral RNA was more than 1 log reduced which is reflected by an increase of the Cₜ value between 26 and 27 at the highest drug concentrations used in the assay.

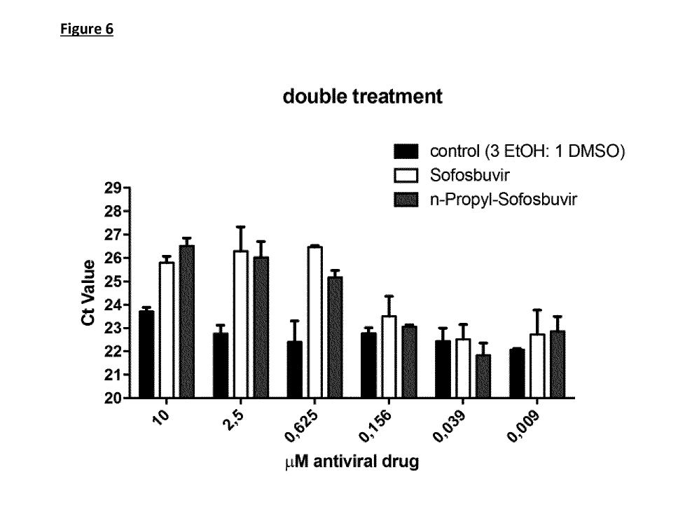

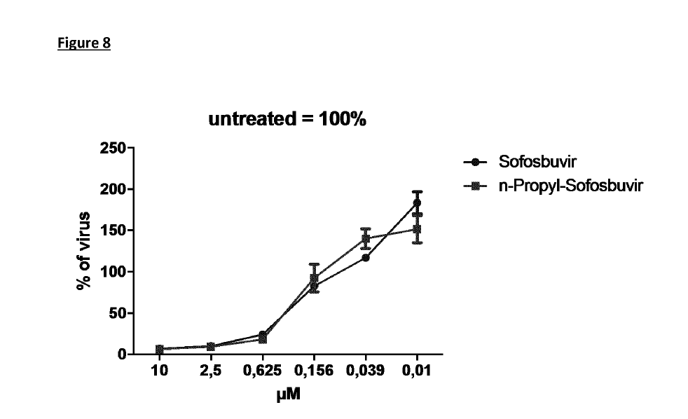

Using CE-labeled PCR HCV quantification kit, we determined the amount of the viral RNA in international Units (IU/ml). As shown in FIG. 7, the estimated reduction of more than 1 log based on the threshold cycles given in FIG. 6 was verified by the quantitative determination of the viral loads. The viral RNA from the controls of around 10⁷ IU/ml was reduced to 6.5×10⁵ IU/ml at the highest concentrations of Sofosbuvir and n-Propyl-Sofosbuvir, corresponding to a reduction to about 95%. The efficacy of the drugs is given in FIG. 8. As expected the compounds are even more effective when given twice compared to the single dose application.

### Permeability Studies

The bi-directional Caco-2 cell permeability assay was performed as described as follows: Caco-2 cells (ECACC) were seeded onto 24-well Transwell plates at 2×105 cells per well and used in confluent monolayers after a 21 day culture at 37° C. under 5% CO2. Test and control compounds (propranolol, vinblastine), prepared in DMSO, were added (10 μM, 0.1% DMSO final, n=2) to donor compartments of the Transwell plate assembly in assay buffer (Hanks balanced salt solution supplemented with 25 mM HEPES, adjusted to pH 7.4) for both apical to basolateral (A>B) and basolateral to apical (B>A) measurements. Incubations were performed at 37° C., with samples removed from both donor and acceptor chambers at T=0 and 1 hour and compound analysed by mass spectrometry (LC-MS/MS) including an analytical internal standard.

Apparent permeability (Papp) values were determined from the relationship: Papp=[CompoundAcceptor T=end]×VAcceptor/([CompoundDonor T=0]×VDonor)/incubation time×VDonor/Area×60×10-6 cm/s

Where V is the volume of each Transwell compartment (apical 125 μL, basolateral 600 μL), and concentrations are the relative MS responses for compound (normalized to internal standard) in the donor chamber before incubation and acceptor chamber at the end of the incubation.

Area=area of cells exposed for drug transfer (0.33 cm2).

Efflux ratios (Papp B>A/Papp A>B) were calculated for each compound from the mean Papp values in each direction. A finding of good permeability B>A, but poor permeability A>B, suggests that a compound is a substrate for an efflux transporter, such as P-glycoprotein.

Lucifer Yellow (LY) was added to the apical buffer in all wells to assess viability of the cell layer. As LY cannot freely permeate lipophilic barriers, a high degree of LY transport indicates poor integrity of the cell layer and wells with a LY Papp>10×10-6 cm/s were rejected. Note that an integrity failure in one well does not affect the validity of other wells on the plate.

Compound recovery from the wells was determined from MS responses (normalized to internal standard) in donor and acceptor chambers at the end of incubation compared to response in the donor chamber pre-incubation. Recoveries <50% suggest compound solubility, stability or binding issues in the assay which may reduce the reliability of a result.

nPropyl-Sofosbuvir (compound I″a) and Sofosbuvir were tested in a bi-directional Caco-2 cell permeability assay. An A>B permeability (transfer from the apical to the basolateral side) with apparent permeability coefficients (Pₐₚₚ) of 4×10⁻⁶ cm/s in the case of Sofosbuvir and 2×10⁻⁶ cm/s for nPropyl-Sofosbuvir was reported. As both Pₐₚₚ values are below 5×10⁻⁶ cm/s (=Pₐₚₚ of vinblastine as reference compound), both compounds are classified as low permeable in the A>B direction.

For both compounds, an efflux ratio larger than 2 (4.6 for Sofosbuvir and 4.4 for nPropyl-Sofosbuvir) was measured, indicating that both compounds are substrates of efflux transporters (active transport pathway from the basolateral to the apical side: B>A).

In summary, Sofosbuvir and nPropyl-Sofosbuvir reported similar properties in the Caco-2 cell assay, as both show a low permeability in the A>B direction and the involvement of efflux transporters (in the B>A direction).

### Compositions Comprising Compound I″a (n-Propyl-Sofosbuvir)

Methods for the Preparation of Compositions Comprising n-Propyl-Sofosbuvir (Compound I″a):

For Examples A-C: The formulation was prepared by blending all components in a free fall blender and thereafter compacted in a FlexiTab S. Optionally; an aqueous suspension of the coating agent was applied in a film-coating process to achieve a target weight gain of 3%.

For Example D: The powder blend was prepared according to the following description. The intragranular components were homogenized in a free fall blender and compacted via a FlexiTab S. The resulting ribbons were milled through a milling screen and thereafter blended with the extragranular excipients. The tablets were produced utilizing a RoTab T tablet press resulting in tablets with a target weight of 1200 mg and an n-Propyl-Sofosbuvir target content of 400 mg. An aqueous suspension of the coating agent was prepared and applied in a film-coating process to achieve a target weight gain of 3%.

Compositions Comprising n-Propyl-Sofosbuvir (Compound I″a):

Example A

| Components | mg/tablet | % w/w |
|---|---|---|
| n-propyl Sofosbuvir | 400 | 33.3 |
| Mannitol | 377 | 31.4 |
| Microcrystalline cellulose | 334 | 27.8 |
| Croscarmellose sodium | 60 | 5.0 |
| Colloidal silicon dioxide | 11 | 0.9 |
| Magnesium stearate | 18 | 1.5 |

Example B

| Components | mg/tablet | % w/w |
|---|---|---|
| n-propyl Sofosbuvir | 400 | 33.2 |
| Mannitol | 334 | 27.7 |
| Microcrystalline cellulose | 344 | 28.5 |
| Croscarmellose sodium | 60 | 5.0 |
| Colloidal silicon dioxide | 12 | 1.0 |
| Magnesium stearate | 20 | 1.7 |
| Coating agent | 35 | 2.9 |

Example C

| Components | mg/tablet | % w/w |
|---|---|---|
| n-propyl Sofosbuvir | 400.0 | 55.6 |
| Mannitol | 124.2 | 17.3 |
| Microcrystalline cellulose | 128.5 | 17.9 |
| Croscarmellose sodium | 32.4 | 4.5 |
| Colloidal silicon dioxide | 3.5 | 0.5 |
| Magnesium stearate | 10.5 | 1.5 |
| Coating agent | 20.9 | 2.9 |

Example D

| Components | mg/tablet | % w/w |
|---|---|---|
| Intragranular | | |
| nPropyl Sofosbuvir | 400.0 | 32.4 |
| Mannitol | 360.0 | 29.1 |
| Microcrystalline Cellulose | 296.0 | 23.9 |
| Croscarmellose Sodium | 30.0 | 2.4 |
| Colloidal Silicon Dioxide | 5.4 | 0.4 |
| Magnesium Stearate | 9.0 | 0.7 |
| Extragranular | | |
| Microcrystalline Cellulose | 60.0 | 4.9 |
| Croscarmellose Sodium | 30.0 | 2.4 |
| Colloidal Silicon Dioxide | 0.6 | 0.0 |
| Magnesium Stearate | 9.0 | 0.7 |
| Coating agent | 36 | 2.9 |

1. A compound of formula (I) 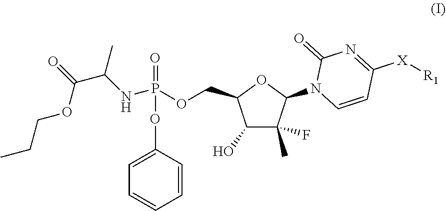
as well as isomers, stereoisomers, diastereoisomers and salts thereof, wherein X is O or
NH and wherein when X is O, R1 is H or a hydroxyl protecting group and when X is
NH, R1 is H or an amine protecting group.

2. The compound of claim 1, wherein X is O and R1 is hydrogen or a hydroxyl protecting group.

3. The compound of any of claim 1, wherein the compound of formula (I) is the compound of formula (I′) or the compound of formula (I″)

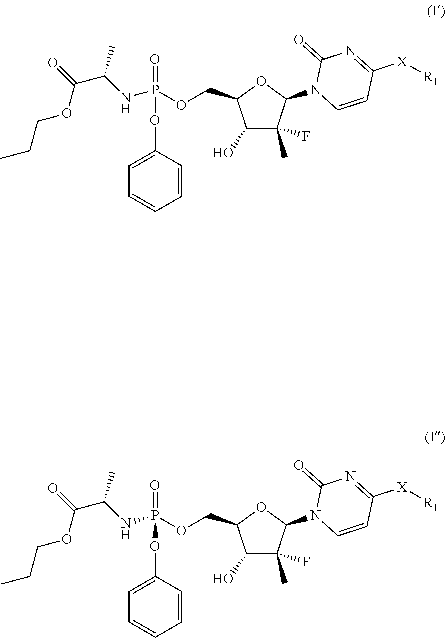

4. The compound of claim 1, wherein the compound of formula (I) is the compound of formula (Ia), the compound of formula (I′a) or the compound of formula (I″a)

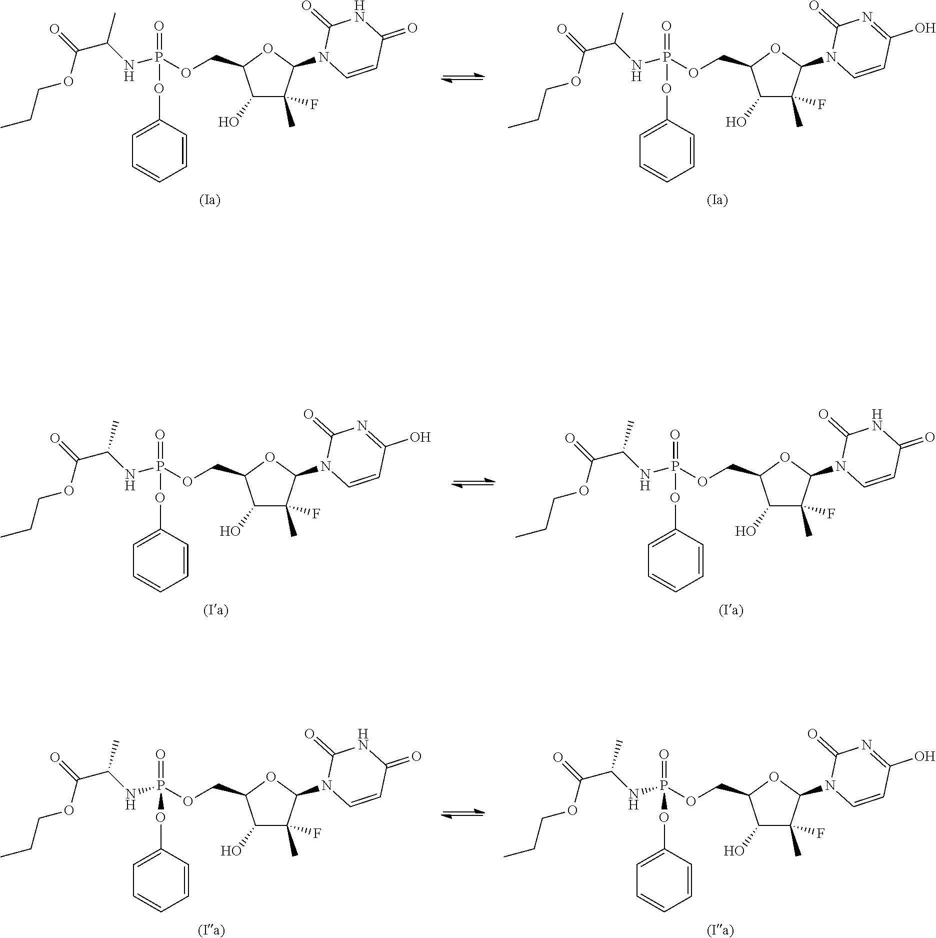

5. The compound of claim 1, wherein the compound of formula (I) is the compound of formula (I″a)

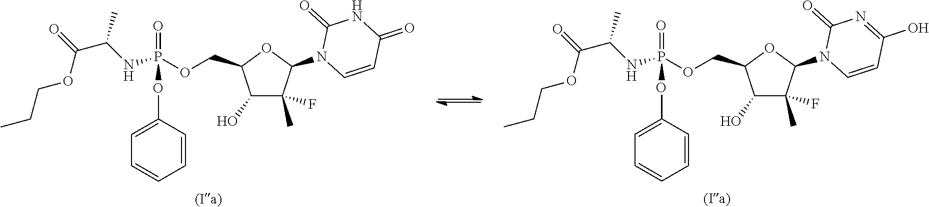 [SMILES: CCCOC(=O)[C@H](C)N[P@](=O)(OC[C@H]1O[C@@H](n2ccc(=O)[nH]c2=O)[C@](C)(F)[C@@H]1O)Oc1ccccc1 ; CCCOC(=O)[C@H](C)N[P@](=O)(OC[C@H]1O[C@@H](n2ccc(O)nc2=O)[C@](C)(F)[C@@H]1O)Oc1ccccc1]

6. The compound of claim 1 in crystalline form.

7. The compound of claim 6 having an X-ray powder diffraction pattern comprising reflections at 2-theta angles of (5.1±0.2)°, (6.9±0.2)°, (9.2±0.2)°, (16.3±0.2)°, (20.4±0.2)° when measured at a temperature in the range of from 15 to 25° C. with Cu-K_{alpha1,2} radiation having a wavelength of 0.15419 nm.

8. The compound of claim 7 comprising further reflections at 2-theta angles of (8.0±0.2)°, (15.3±0.2)°, (16.7±0.2)°, (17.9±0.2)°, (25.6±0.2)° when measured at a temperature in the range of from 15 to 25° C. with Cu-K_{alpha1,2} radiation having a wavelength of 0.15419 nm.

9. The compound of any of claim 6 having a monoclinic space group symmetry and the following unit cell parameters as determined by an X-ray single crystal structure analysis at 173K:
a=12.8656 Angstrom
b=6.0028 Angstrom
c=17.5417 Angstrom
α=90°
β=98.397°
γ=90°.

10. The compound of claim 6 having a melting point in the range of from 77.5° C. to 82.7° C. when measured via differential scanning calorimetry at a heating rate of 10K/min.

11. A process for the preparation of a compound of formula (I) comprising
(i) providing a compound of formula (II) or a mixture comprising the compound of formula (II)
(ii) reacting the compound of formula (II) with a compound of formula (III) to get a compound of formula (I)
(iii) optionally isolating the compound of formula (I) 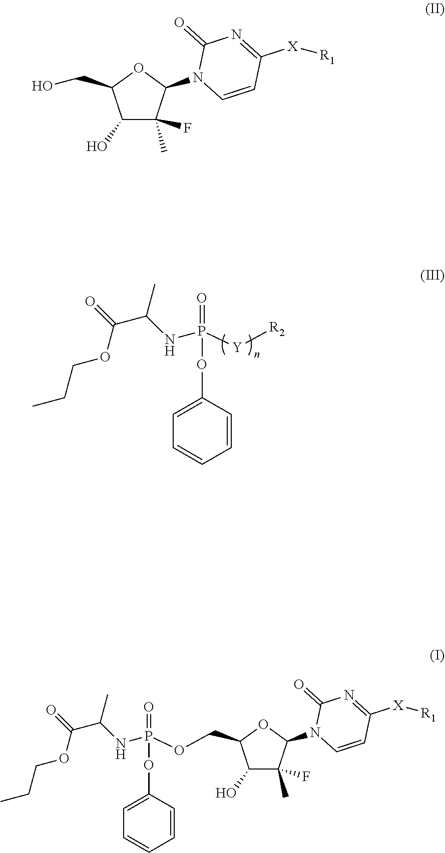
wherein (Y)ₙR₂ is a suitable leaving group for a nucleophilic substitution reaction.

12. The process of claim 11, wherein n is 1, Y is O or S and R₂ is

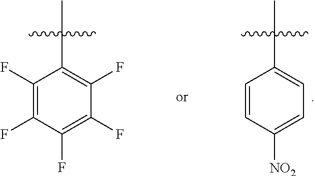 [SMILES: *c1c(F)c(F)c(F)c(F)c1F ; *c1ccc([N+](=O)[O-])cc1]

13. The process of claim 11, wherein R₂ is

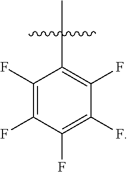 [SMILES: *c1c(F)c(F)c(F)c(F)c1F]

14. The process of claim 11, wherein n is 1, Y is O and R₂ is

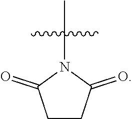 [SMILES: *N1C(=O)CCC1=O]

15. The process of claim 11, wherein n is 0 and R₂ is Cl.

16. The process of claim 11, wherein X is O and R₁ is hydrogen.

17. The process of claim 11, wherein the compound of formula (I) is the compound of formula (Ia), the compound of formula (I′a) or the compound of formula (I″a)

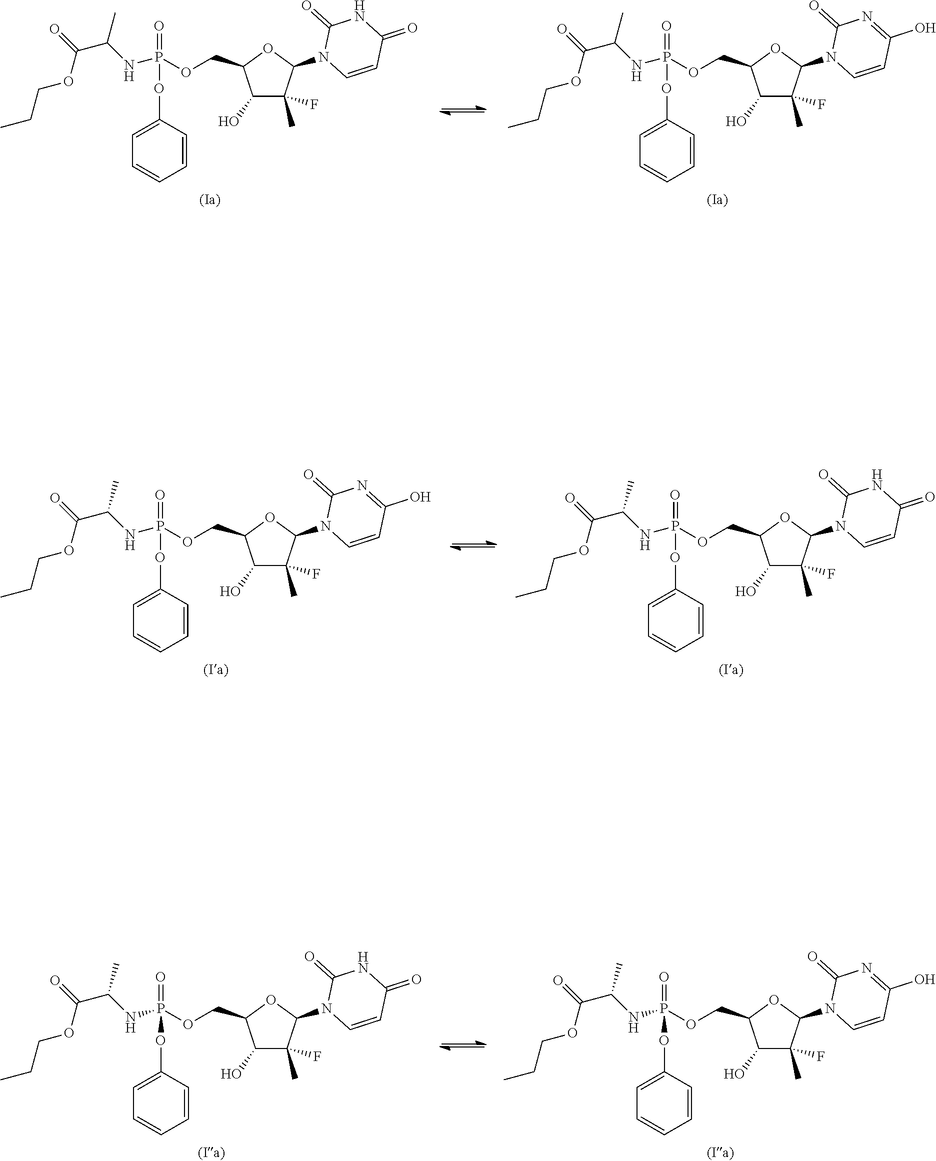

18. A process for the preparation of a compound of formula (I″a) in crystalline form comprising
(i) providing a solution of the compound of formula (I″a) in a suitable solvent or solvent mixture,
(ii) subjecting the solution of (i) to crystallization conditions
(iii) isolating the crystalline compound of formula (I″a)

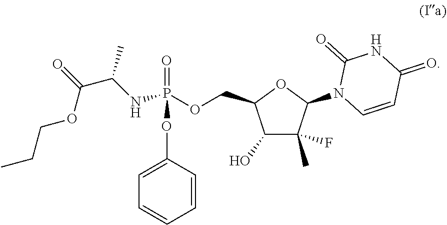 [SMILES: CCCOC(=O)[C@H](C)N[P@](=O)(OC[C@H]1O[C@@H](n2ccc(=O)[nH]c2=O)[C@](C)(F)[C@@H]1O)Oc1ccccc1]

19. The process of claim 18, wherein the solvent or solvent mixture in (i) comprises one or more solvents selected from dichloromethane and ethyl acetate.

20. The process of any of claim 18, wherein subjecting the solution of (i) to crystallization conditions in (ii) comprises adding a further solvent or solvent mixture.

21. The process of claim 20, wherein the further solvent or solvent mixture consists of or comprises pentane, hexane, heptane, diisopropyl ether, or mixtures thereof.

22. The process of any of claim 20, wherein the further solvent or solvent mixture comprises heptane.

23. The process of claim 20, wherein the further solvent or solvent mixture is added in a volume ratio of from 30:30 to 10:60 preferably of from 20:70 to 20:30, preferably of from 25:115 to 55:55 relative to the volume of the solvent or solvent mixture provided in (i).

24. A compound of formula (III) 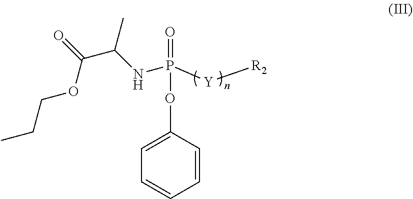
wherein (Y)ₙR₂ is a suitable leaving group for a nucleophilic substitution reaction.

25. The compound of claim 24, wherein n is 1, Y is O and R2 is

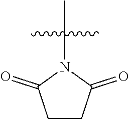 [SMILES: *N1C(=O)CCC1=O]

26. The compound of claim 24, wherein n is 0 and R2 is Cl.

27. The compound of claim 24, wherein the compound of formula (III) is the compound of formula (III′) or the compound of formula (III″) [chemical structure]
